SEQUENCE LISTING (54 sequences)

SEQ ID NO: 1 - protein, 473 aa, Neorickettsia helminthoeca
MICNIAKILFISTLLTSPVYASVENPSIGTRPPLEGKSCGCKKTCGCKKTCGCSKNVHTGTSSGHNTINQPSFTIKGSSVFSFHYGKNEDFFELSKNLLKIKNLPHSGTPTSASDVKPLYNVGISGEYDRPNKILSKSRISIEARRKMADFSYGVLLEPMFDMSKTVSTRNAYIFLEAPYGRFEMGQVNDSATSALKIDASSVAATGAGIRDLDWTEVANLEGRPEHAVFDTSTSSTQHKRHKNVTHPFLVHPNYYVAYDAPIRANFTTTGLGAFKLAVSYTNRTADGIYRDILDFGCGYTGIAKNLNYGVSITGQTSLIEPTGNLHHPLKRFEIGGMAEMYGIKLAGSFGNSFLSGIKINKNMQLDLSKGIDDPKQFVSTNGQLTYMTLGTAFESGPMMFSVNYMKSDNMLKKSDKSTLHVISIGTHYRLTGEAYELTPYVSGRYFVTSEAGVPKGDNNKGYVISSGLKVSY

SEQ ID NO: 2 - protein, 333 aa, Neorickettsia helminthoeca
MANGVTLFDILSNDTNFNTLTDSTVLDLLKHDTSSNTLKDTTAAEVLKNTTAGDILKNSTAAEVLKNTTAGDILKNSTAAEVLKNTTAGDILKNSTAAEVLKDANAKNVLENANAAAVLKDLGAAGTLKDATAAGALKDSEIQGLLKDKTAVDLLKNASLCGVLKNNAEARNLLKETDFQNLLKDQTAAGALKDSEIQGLLKDKTAVDSLERAIVRDTLKCKDAAIVLQDEGFSALLRDNVNTEARNLLKETDFQNLLKDQTAAGALKDSTIQGLLKDAAAIGALKQSGISELLKDTNAKRFLEDSAFQASLKACESSSELQNRLKEITIPKK

SEQ ID NO: 3 - protein, 260 aa, Neorickettsia helminthoeca
MLGCRIAILLSLLLFLSPAEALFGINANTGFYISGGYGALMSGKAGVDNAATYANQAAQKFRSVSKDHLLHEDLKNFNVAAGFSILGFSLDVEGLYGYLESAKTSKNGTLKLKLPEKVGDQEFSYFLGFVNANLEFSGAALLNPYVGLGIGTGTVTFAIENKDSDRRYGFPLATQIKAGLALDLGSYFFVSLKPYIGYRMLMVSSTGVDTLSVVPTLIPTQNANPDAGIAGRIKEVVTAISDISHTSHNAEIGIKIQLGI

SEQ ID NO: 4 - protein, 315 aa, Neorickettsia helminthoeca
MINSSFLRKALLLSCLFAMPLSGNSAAKVEEAANAGVYGRIFQLSKVSGETNFMDTGRHYHHAVSEDVASLIKDSQHGPLLYHDGGVFGDYRPTHALNMVGGGFALGYRTQNARFEFEGIINGEGKLSDSAESQFYGLAAVPAEVTKDGKVNGQDHEGSGCKYLKGVKNVAVGPMNFSKFSYAATLFNIYQDIPTGDVMKLYVGGGVGISRVTYNLTSTQNLVSTPFVAQGKVGVTFDVGDLGSMGMVPYLGYSALYFAEKEANSRVTGLTSHKMSKDKKGPCDKKDGIPGLEFAPVAKHLLHNIEFGVTFSLDA

SEQ ID NO: 5 - protein, 227 aa, Neorickettsia helminthoeca
MINKKFLISVALAGVASTSDAQDALEDADIFYAKVGYNATKMQPVEWTKARVSGDTSKFKPEYESSFIGGSAALGYYFGGMRVELEGSMYNVDSKKGSKIPETKQPDAPAIKYGGACFMGGMLSVNYDVALTDYISPYFGVGFGLSRVSLKLDDDALSTAYHMSSQLKGGVSITGLAAVVPYAGYKFTYMNDKGYSKVALANSTELAPQLSHMVHNFEAGLMLPMNA

SEQ ID NO: 6 - DNA, 1422 bp, Neorickettsia helminthoeca
atgatatgcaacatcgctaaaattctattcatttctacattgctcacaagtcctgtatacgcttctgtagagaacccatcaattggaacaagaccacctctagaagggaaaagctgtggatgtaagaaaacttgtggatgtaagaaaacttgtggatgtagcaaaaatgtccatacaggtacttcttctggtcataatacaataaatcaaccatctttcacaataaagggaagtagtgttttctcgttccactatgggaagaatgaagattttttcgaacttagtaaaaacctattgaaaatcaagaaccttccgcacagtggaacaccaactagcgctagtgatgttaaacccctatataacgtaggtatctcaggtgagtatgaccgtccaaataaaatcctcagcaaaagtaggatatcaatcgaggcaagacgtaaaatggcagacttctcttatggagttctgctagaaccgatgttcgatatgagtaaaacagtcagcaccaggaacgcatatatcttccttgaagcaccgtatggaagatttgagatgggccaagttaatgatagcgcaacctcagcactgaaaattgatgcatcgtcagttgcagctaccggcgcaggaatcagagatttggattggactgaagtcgcaaaccttgaaggaaggcctgaacacgctgtatttgataccagcactagtagcacacagcataaaagacataaaaatgtaactcacccgttcttggtccacccgaattattatgtagcatatgatgctccaatcagagcgaatttcaccactactggactcggcgcattcaaattagcagtgagttacacaaacagaactgctgatggaatatatcgcgatattttggatttcggttgtggatatactggaattgcaaagaatctgaactatggtgtttccatcactgggcaaaccagcctcatagagccaactggaaatctgcaccatcctctaaagagattcgagattggcggaatggcagagatgtatggtatcaagcttgcaggatcatttggcaattctttcctttctggaattaaaataaataaaaacatgcaacttgatctctcaaagggtatagatgatccaaagcaatttgtcagtacaaacggtcaacttacctatatgacattaggtacagcattcgaaagtggcccaatgatgttcagtgtcaactacatgaagagcgataatatgttgaaaaaatccgacaaaagtacattgcatgttatttctattggaacacactaccgcttaacaggagaagcatatgaactcactccttatgtgagtggaagatattttgtcacctcagaagctggtgtaccaaaaggtgataataacaaaggttatgtaatttcttcaggtctcaaagtatcatattga

SEQ ID NO: 7 - DNA, 1002 bp, Neorickettsia helminthoeca
atggcaaacggtgtcacactatttgatattttgtcaaatgacactaattttaacaccttaaccgatagtacggtccttgatctgcttaagcatgatacctcaagtaatacattaaaagatacaaccgcagctgaggtattaaaaaatacaactgctggagatatattaaagaattcaaccgcagctgaggtattaaaaaatacaactgctggagatatattaaagaattcaaccgcagctgaggtattaaaaaatacaactgctggagatatattaaagaattcaaccgcagctgaggtactaaaagatgcaaatgcaaaaaatgtactggaaaacgcaaatgcagctgcggtattaaaagatttaggcgcggcggggaccctaaaagatgcaacagcagcaggtgccttaaaagattcagaaattcagggcttgttaaaggataagaccgcggtagaccttttaaagaatgcaagtctctgcggagtgttaaaaaacaatgcagaagctagaaaccttttgaaagagacagacttccagaatctattaaaggatcagacagcagcaggtgccttaaaagattcagaaattcagggcttgttaaaggataagaccgcggtagacagcttagaaagggcgattgttcgggatacgctaaagtgcaaagacgcagcaatcgttttgcaagatgaaggattcagcgctctattacgagataatgtcaatacagaagctagaaaccttttgaaagagacagacttccagaatctattaaaggatcagacagcagcaggtgccttaaaagattcaacaattcagggcctattaaaggatgcagctgcgataggggctttaaaacaatcgggtatttctgagttgttgaaggatactaatgccaagagattcttagaggatagtgccttccaagcctcattaaaggcttgtgagagctcaagtgagctacagaatagacttaaagagataactatccccaaaaaataa

SEQ ID NO: 8 - DNA, 783 bp, Neorickettsia helminthoeca
atgctcggatgtcgtatcgctattttgctgtctctgctactctttttgagtcctgctgaggcgcttttcggaataaacgcgaacaccgggttttacatcagtggtggatatggcgctttgatgtctggcaaggcgggtgttgataatgctgccacttatgcaaatcaagcagctcagaaatttagaagtgtgagcaaggatcatctgcttcacgaggatctgaagaacttcaatgtggcagctgggttttcaattttaggattctcattggacgttgaaggtctctatggatatcttgaatctgcgaaaacaagtaaaaacggtaccctcaaactcaaattgccagaaaaagttggtgatcaggaattttcctattttcttggctttgttaacgcgaatctggaattctcaggagcggcgttattgaatccctacgttggattaggtatcggcaccgggactgtcacattcgctattgagaataaggattcggataggagatacggatttcctctggcgacgcagataaaagctggcttagcgcttgatctaggatcctatttctttgtctcattgaagccgtatattggttatcggatgctgatggtctctagtacgggagtcgatacactttccgttgtccctacactcattccgacgcagaatgcaaatcctgatgcaggaatagctggtaggatcaaggaagttgtcactgcaatcagtgatattagtcacacctcgcataatgctgagattggaatcaagatccagcttggaatataa

SEQ ID NO: 9 - DNA, 948 bp, Neorickettsia helminthoeca
atgattaatagtagttttttgagaaaggcattactcctctcctgtttgtttgcgatgccgctgagtggcaacagtgctgccaaagtagaagaagcggcgaatgcaggtgtttatggtagaattttccagctaagcaaggttagcggcgaaactaattttatggacactgggcgccattaccaccatgcagttagtgaagatgttgctagcctgattaaagattcacagcatggcccattattataccacgatggtggcgtttttggagactacaggcctacacatgcacttaacatggtaggtggtggttttgcacttggataccgcacccaaaacgcaaggtttgagtttgaagggataataaacggcgaaggtaaactaagtgacagcgctgaatcacagttttatggtcttgctgctgtaccagctgaggtaaccaaagatggtaaagtaaatggacaggaccatgagggatcaggatgtaagtacctcaaaggcgtgaagaatgtggcggttggcccaatgaactttagtaagttctcttatgcggctaccctgtttaatatctatcaggatattccaactggagatgtaatgaaattgtatgtaggcggtggtgtcggaataagccgtgttacttacaacttgacaagtactcaaaaccttgttagcactccatttgttgcgcagggtaaggtcggtgtaacctttgatgtcggcgatctaggaagtatgggcatggtaccatatcttggctactcagcgctctacttcgctgaaaaagaagctaatagtcgcgtgacaggtctaactagccacaaaatgagcaaggataaaaagggcccttgcgacaagaaagatggtatcccaggacttgagtttgcgcctgtggcaaaacacttgctacataacattgagtttggggttactttttcacttgacgcctga

SEQ ID NO: 10 - DNA, 693 bp, Neorickettsia helminthoeca
atgataaataaaaagttcctaataagcgtggctcttgcaggtgttctttgccttgcatctacctcagatgcgcaagatgccctagaggatgcagatattttctatgccaaagttgggtataacgctaccaaaatgcagccggtggagtggactaaggcccgcgtatcgggtgatactagtaaattcaagccagagtatgaaagtagtttcattggcggtagtgctgctctcggatattacttcggtggcatgagagtcgaactggaaggcagcatgtataatgttgattctaaaaaaggttctaaaatacctgaaactaagcagcccgatgcacctgctataaagtatggtggcgcttgttttatgggtggcatgctttcagtaaactacgatgtggctctaactgattatatcagcccgtactttggagtaggtttcggtctaagcagagtatccctaaagcttgatgatgatgcattgtctactgcgtatcatatgtcatcccaattgaaaggtggtgtaagcatcactgggctcgctgctgtggtcccttatgctggatataagttcacatatatgaatgacaaaggttattcaaaagtagctcttgctaatagtactgagcttgctccgcaactttctcatatggtgcacaactttgaggctggtctaatgctacctatgaatgcgtaa

SEQ ID NO: 11 - protein, 10 aa, Neorickettsia helminthoeca
IFLCSCLKDS

SEQ ID NO: 12 - protein, 10 aa, Neorickettsia helminthoeca
FGSYACFVIP

SEQ ID NO: 13 - protein, 10 aa, Neorickettsia helminthoeca
SLLLSCVFSG

SEQ ID NO: 14 - protein, 10 aa, Neorickettsia helminthoeca
LGLVTCLSSK

SEQ ID NO: 15 - protein, 10 aa, Neorickettsia helminthoeca
FSLSSCAKRG

SEQ ID NO: 16 - protein, 10 aa, Neorickettsia helminthoeca
SSLAHCTSAI

SEQ ID NO: 17 - protein, 10 aa, Neorickettsia helminthoeca
LFLDPCLAEN

SEQ ID NO: 18 - protein, 10 aa, Neorickettsia helminthoeca
CSSADCSHTS

SEQ ID NO: 19 - protein, 10 aa, Neorickettsia helminthoeca
LCFAPCHSLE

SEQ ID NO: 20 - protein, 10 aa, Neorickettsia helminthoeca
FTFSGCDHCE

SEQ ID NO: 21 - protein, 10 aa, Neorickettsia helminthoeca
FLFNGCDIEC

SEQ ID NO: 22 - protein, 10 aa, Neorickettsia helminthoeca
LLMSGCFKKG

SEQ ID NO: 23 - protein, 10 aa, Neorickettsia helminthoeca
LVVSGCTPGK

SEQ ID NO: 24 - protein, 33 aa, Neorickettsia helminthoeca
AGPRGEDARANVGDPNLPRSSSLPNPNVSHGQE

SEQ ID NO: 25 - protein, 20 aa, Neorickettsia helminthoeca
TRSHGDLTEMRKALSREPSP

SEQ ID NO: 26 - protein, 25 aa, Neorickettsia helminthoeca
VEVQTDAPEEPERSTGAASTQTMSE

SEQ ID NO: 27 - protein, 21 aa, Neorickettsia helminthoeca
PIPSAEVAQQPAAEPVQQATE

SEQ ID NO: 28 - protein, 44 aa, Neorickettsia helminthoeca
VEQGSDDNTGADNIEEAIEPIPPAEVAQQPAAEPVQQATEPIPS

SEQ ID NO: 29 - protein, 35 aa, Neorickettsia helminthoeca
GEISTGPEAITEATEVQDEVKLNPEVITEASGIVD

SEQ ID NO: 30 - protein, 33 aa, Neorickettsia helminthoeca
DRVSDAMPGIQKGAKSTVVWTADAAGRVGAVML

SEQ ID NO: 31 - protein, 35 aa, Neorickettsia helminthoeca; identical to SEQ ID NO: 29

SEQ ID NO: 32 - protein, 7 aa, Neorickettsia helminthoeca
FPHKVFS

SEQ ID NO: 33 - protein, 8 aa, Neorickettsia helminthoeca
EIMNTTNK

SEQ ID NO: 34 - protein, 36 aa, Neorickettsia helminthoeca
SSTGSCRPIAAPILNGASLHGVYTSLFEGNKDPGTV

SEQ ID NO: 35 - protein, 75 aa, Neorickettsia helminthoeca
LRKVGIKEKPFTGDDLIAELKARIEKRSEKNPGKPTVSDSRKRMVTSDAKDSKQRETQGEKSGNPRTITTETTLE

SEQ ID NO: 36 - protein, 37 aa, Neorickettsia helminthoeca
VPATSAVMKSIASTGEGGEVVGLSPTLTKFLKEVGEV

SEQ ID NO: 37 - protein, 30 aa, Neorickettsia helminthoeca
AKYYSAHRDEILQRRRESRARDPERFCEYG

SEQ ID NO: 38 - protein, 30 aa, Neorickettsia helminthoeca
PEKFREYKAKHYSAHRDEILQRRRESRARD

SEQ ID NO: 39 - protein, 30 aa, Neorickettsia helminthoeca
KYYSAHRDEILQRRRESRARDPEKFRGYGA

SEQ ID NO: 40 - protein, 74 aa, Neorickettsia helminthoeca
KKKAEQPIQGTSSSSAPGPSTADLSTSSGSTTVLAPKRRKLTPEEKRERNRISQAKYYSAHRDEIIQRQREQRA

SEQ ID NO: 41 - protein, 30 aa, Neorickettsia helminthoeca
ERFREYKAKHYSAHRDEILQRRRESRARDP

SEQ ID NO: 42 - protein, 47 aa, Neorickettsia helminthoeca
KPKTGEGMVENPIYESGDPVQGAESTENPYSLRGAEGQEEPIYATVD

SEQ ID NO: 43 - protein, 18 aa, Neorickettsia helminthoeca
AAEVLKNTTAGDILKNST

SEQ ID NO: 44 - protein, 56 aa, Neorickettsia helminthoeca
NAPPESLQIELTLDQSEDSSEKQPITPPQQTEPVSLQHQIEPTAPPEPHKTEPVIV

SEQ ID NO: 45 - DNA, 32 bp, Artificial Sequence
  synthetic construct
ataggccatggcttctgtagagaacccatcaa

SEQ ID NO: 46 - DNA, 36 bp, Artificial Sequence
  synthetic construct
ctagagaattcgtatatgatactttgagacctgaag

SEQ ID NO: 47 - DNA, 30 bp, Artificial Sequence
  synthetic construct
ataggccatggcgcttttcggaataaacgc

SEQ ID NO: 48 - DNA, 37 bp, Artificial Sequence
  sythetic construct
ctagagaattcaatattccaagctggatcttgattcc

SEQ ID NO: 49 - DNA, 36 bp, Artificial Sequence
  synthetic construct
ataggccatggccaaagtagaagaagcggcgaatgc

SEQ ID NO: 50 - DNA, 34 bp, Artificial Sequence
  synthetic construct
ctagagcggccgcggcgtcaagtgaaaaagtaac

SEQ ID NO: 51 - DNA, 32 bp, Artificial Sequence
  synthetic construct
ataggccatggcgcaagatgccctagaggatg

SEQ ID NO: 52 - DNA, 31 bp, Artificial Sequence
  synthetic construct
ctagagcggccgcattcataggtagcattag

SEQ ID NO: 53 - DNA, 36 bp, Artificial Sequence
  synthetic construct
ataggccatggatctgcttaagcatgatacctcaag

SEQ ID NO: 54 - DNA, 42 bp, Artificial Sequence
  synthetic construct
ctagagcggccgcttttttggggatagttatctctttaagtc

(19) United States
(12) Patent Application Publication
(10) Pub. No.: US 2017/0281740 A1
(43) Pub. Date: Oct. 5, 2017

(54) SERODIAGNOSIS OF SALMON POISONING DISEASE

(71) Applicant: Ohio State Innovation Foundation, Columbus, OH (US)

(72) Inventors: Yasuko Rikihisa, Worthington, OH (US); Mingqun Lin, Columbus, OH (US)

(21) Appl. No.: 15/476,011

(22) Filed: Mar. 31, 2017

Related U.S. Application Data
(60) Provisional application No. 62/316,254, filed on Mar. 31, 2016.

(51) Int. Cl. A61K 39/00; G01N 33/569

(52) U.S. Cl. CPC A61K 39/0003; G01N 33/56911; G01N 2800/26; G01N 2333/29; A61K 2039/552

(57) ABSTRACT

Neorickettsia helminthoeca is an obligate intra-cytoplasmic bacterium that causes salmon poisoning disease (SPD), an acute, febrile, fatal disease of dogs. Disclosed are compositions and methods for the immunodetection of N. helminthoeca in a canine subject. Also disclosed are immunogenic N. helminthoeca peptides that can be used in a vaccine for N. helminthoeca.

### CROSS REFERENCE TO RELATED APPLICATIONS

This application claims the benefit of U.S. Provisional Patent Application Ser. No. 62/316,254 filed Mar. 31, 2016, the disclosure of which is expressly incorporated herein by reference.

### FIELD

The present invention relates to compounds, compositions, and methods for the serodiagnosis of salmon poisoning disease (Neorickettsia helminthoeca).

### BACKGROUND

Salmon poisoning disease (SPD), an acute and often-fatal illness in wild and domestic canids, was first discovered in the 1800s when early settlers in Pacific Northwest noted their dogs becoming ill following ingestion of salmon (Philip, 1955). In 1950, a bacterial pathogen was implicated as the causative agent of SPD and named Neorickettsia helminthoeca, due to its biological similarity to the members of the family Rickettsiaceae and the novel invertebrate/helminth vector (Cordy and Gorham, 1950; Philip, 1955). N. helminthoeca exists in all life stages of the fluke Nanophyetus salmincola (Bennington and Pratt, 1960; Schlegel et al., 1968), which has a complicated digenetic life cycle involving both pleurocid fresh water snails (Oxyfrema silicula) and salmonid fish as intermediate hosts (Millemann and Knapp, 1970; Headley et al., 2011). Due to the limited geographic range of the vector and intermediate hosts, the distribution of SPD was thought to be limited to the northern Pacific coast. However, SPD cases have been confirmed in Southern California (this study) (Veterinary Practice News, 2009), Vancouver Island, Canada (Booth et al., 1984) and Maringa, Brazil using immunohistochemical, histopathological and molecular diagnostic techniques (Table 1) (Headley et al., 2004; Headley et al., 2006; Headley et al., 2011), though the vector and life cycle in these regions remain to be identified. The expansion of the geographic distribution of SPD where N. salmincola has not been documented suggests the potential adaptation of this organism to other trematode vectors.

While there is a large range of definitive hosts for the trematode, N. helminthoeca causes severe SPD in members of the Canidae family including dogs, foxes, and coyotes (Cordy and Gorham, 1950; Philip et al., 1954a; Philip et al., 1954b; Philip, 1955; Foreyt et al., 1987). Dogs most commonly acquire SPD when they eat raw or undercooked salmonid fish containing encysted trematodes injected with N. helminthoeca. Upon ingestion, the metacercariae stage of the trematode matures in the intestinal lumen for 5-8 days and releases the bacteria to be picked up by monocytes and macrophages in the intestinal wall. The exact mechanism of bacterial entry into these cells is not known, but morphological studies demonstrate the organism existing as clusters termed morulae or singly within a host cell-derived membrane vacuole in the cytoplasm of the canine host cell (Rikihisa et al., 1991). N. helminthoeca-infected cells travel throughout the circulation and accumulate in the thoracic and abdominal lymph nodes with the mesenteric and ileocecal lymph nodes being most commonly affected (Philip et al., 1954a; Philip, 1955; Headley et al., 2011). Symptoms begin with pyrexia (39.8-40.9° C.) that persists for 6-7 days and anorexia (Rikihisa et al., 1991). Dogs progress to vomiting and diarrhea that may or may not contain blood 4-6 days following development of a fever. Other symptoms include ocular discharge, weight loss, lethargy, and dehydration. If left untreated, death occurs 2-10 days after development of symptoms (Philip, 1955). Current therapies for SPD include fluid therapy, blood transfusions for hemorrhagic diarrhea, anti-helminthic praziquantel, and oral doxycycline or intravenous oxytetracycline. Affected individuals produce specific immunity to SPD following recovery from the disease (Philip et al., 1954a; Philip, 1955).

Neorickettsia species are obligatory intracellular α-proteobacteria that belong to the family Anaplasmataceae in the order Rickettsiales (Rikihisa et al., 2005). Neorickettsia spp. are the deepest branching lineage in the family Anaplasmataceae, whereas Anaplasma and Ehrlichia are sister genera that share a common ancestor with Wolbachia spp. (FIG. 1) (Pretzman et al., 1995; Wen et al., 1995; Wen et al., 1996). The branching pattern suggests that the speciation of N. helminthoeca occurred earlier than the speciation of N. risticii and N. sennetsu. These findings and many other molecular phylogenetic analyses (Anderson et al., 1992; Wen et al., 1995; Wen et al., 1996; Rikihisa et al., 1997) led to the drastic reclassification of the family Anaplasmataceae (Dumler et al., 2001).

Currently, only three pathogenic species of Neorickettsia, namely N. helminthoeca (type species), N. sennetsu (agent of human Sennetsu fever), and N. risticii (agent of Potomac horse fever) have been culture isolated and characterized in sufficient details with documented biological and medical significance (Table 1) (Rikihisa et al., 1991; Rikihisa et al., 2005). All of them are known to transmit from trematodes to monocytes/macrophages of mammals (dogs, humans, and horses, respectively) and cause severe, sometimes fatal illnesses (Table 1) (Rikihisa et al., 2005). In addition, the Stellantochasmus falcatus (SF) agent, which is closely related to N. risticii, was culture isolated from S. falcatus fluke encysting the grey mullet fish in Japan (Wen et al., 1996) and from fish in Oregon (Rikihisa et al., 2004). The initial 16S rRNA gene sequence-based phylogenetic analysis of N. helminthoeca revealed that the divergence of 16S rRNA sequences is around 5% between N. helminthoeca and N. risticii or N. sennetsu, whereas it is only 0.7% between N. risticii and N. sennetsu.

As endosymbionts of digenetic trematodes (parasitic flatworms or flukes), Neorickettsia species are abundant in nature and have been identified throughout the life cycle of the trematodes and the hosts of trematodes including the essential first intermediate host of snails, the second intermediate hosts such as fish and aquatic insects, and the definitive hosts such as mammals and birds wherein the trematodes sexually reproduce fertilized eggs (Cordy and Gorham, 1950; Philip et al., 1954a; Philip et al., 1954b; Philip, 1955; Foreyt et al., 1987; Gibson et al., 2005; Rikihisa et al., 2005; Gibson and Rikihisa, 2008; Greiman et al., 2016). Recent reports revealed more than 10 new genotypes of Neorickettsia in divergent digenean families throughout the world, including Asia, Africa, Australia, Americas, and even Antarctica (Ward et al., 2009; Tkach et al., 2012; Greiman et al., 2014; Greiman et al., 2017), suggesting a global distribution of Neorickettsia spp. Notably, a Neorickettsia sp. was found in the medically important trematode Fasciola hepatica (the liver fluke, fasciolosis disease agent) isolated from a sheep in Oregon US (McNulty et al., 2017). In addition, a related new species named Candidatus “Xenolissoclinum pacificiensis L6” was identified in the ascidian tunicate Lissoclinum patella, a marine chordate animal at the coast of Papua New Guinea (Kwan and Schmidt, 2013), implicating even boarder distribution of Neorickettsia-like bacteria among diverse invertebrates. To date, the complete genome sequences have been determined only for N. sennetsu (Dunning Hotopp et al., 2006) and N. risticii (Lin et al., 2009), and almost complete genome sequences were obtained for Neorickettsia endobacterium of F. hepatica (NFh) and Candidatus “X. pacificiensis” (Kwan and Schmidt, 2013; McNulty et al., 2017). The phylogenetic analysis based on 16S rRNA gene sequences suggests that NFh shares >99% identity with N. risticii and N. sennetsu, while Candidatus “X. pacificiensis” is distantly related to Neorickettsia spp. (FIG. 1). Genomic comparisons indicated that approximately 97% of the predicted proteins (721 out of 744) of NFh showed top matches to N. risticii or N. sennetsu, while 22 unique proteins of NFh were hypothetical proteins without functional annotations (McNulty et al., 2017).

Because the mortality rate of SPD is >90% without rapid antibiotic treatment (Philip, 1955; Rikihisa et al., 1991), the current inefficient diagnostic method (fecal examination for parasite eggs and/or Romanowsky staining of lymph node aspirates), and the expansion of the geographic distribution of SPD, there remains a need for more rapid, sensitive, and specific serodiagnostic technique, as well as an effective vaccine.

### SUMMARY

As disclosed herein, the genome of N. helminthoeca Oregon consists of a small, single circular chromosome of 884,232 bp and encodes 37 RNA species and 774 proteins. Although N. helminthoeca has a very limited capacity to synthesize amino acids and lacks many metabolic pathways, it is capable of making all major vitamins, cofactors, and nucleotides, which may be beneficial to the trematode host. Like other members of the family Anaplasmataceae, helminthoeca lacks genes for lipopolysaccharide biosynthesis. However, peptidoglycan biosynthesis pathway is conserved, suggesting its mechanical strength and inflammatory potential. Genes potentially involved in the pathogenesis of N. helminthoeca were identified, including putative outer membrane proteins, two-component systems, type I and IV secretion systems, and putative transcriptional regulators. Five predicted major surface antigens P51, NSP-1/2/3, and SSA of N. helminthoeca were cloned and expressed and reactivity of both experimentally and naturally infected dog blood specimens to these antigens were evaluated. The result showed strong antigenicity. These findings provide the tools with which to design rapid and sensitive serodiagnostic methods and new prevention strategies for Salmon poisoning disease.

Therefore, disclosed is an immunogenic composition comprising one or more isolated Neorickettsia helminthoeca proteins, or immunogenic fragments or variants thereof, or a fusion protein containing same, and a pharmaceutically acceptable carrier, wherein said composition is capable of producing antibodies specific to N. helminthoeca in a subject to whom the immunogenic composition has been administered, and wherein the isolated N. helminthoeca protein is selected from the group consisting of: SEQ ID NO:1, SEQ ID NO:2, SEQ ID NO:3, SEQ ID NO:4, and SEQ ID NO:5.

In one aspect, disclosed herein is a method of preventing or inhibiting salmon poisoning disease (SPD) in a subject comprising:

administering to the subject an immunogenic composition comprising one or more isolated Neorickettsia helminthoeca proteins, or immunogenic fragments or variants thereof, or a fusion protein containing same, and a pharmaceutically acceptable carrier,

wherein said composition is administered in an amount effective to prevent or inhibit salmon poisoning disease (SPD), and

wherein the isolated N. helminthoeca protein is selected from the group consisting of: SEQ ID NO:1, SEQ ID NO:2, SEQ ID NO:3, SEQ ID NO:4, and SEQ ID NO:5.

In some embodiments, the isolated helminthoeca protein is SEQ ID NO:1. In some embodiments, the isolated N. helminthoeca protein is SEQ ID NO:2, In some embodiments, the isolated N. helminthoeca protein is SEQ ID NO:3. In some embodiments, the isolated N. helminthoeca protein is SEQ ID NO:4. In some embodiments, the isolated N. helminthoeca protein is SEQ ID NO:5.

In some embodiments, the subject is a member of the Canidae family

Also disclosed is a method for detecting Neorickettsia helminthoeca infection in a canine subject, comprising assaying a sample from the subject for antibodies specific for a N. helminthoeca protein selected from the group consisting of P51, NSP1, NSP2, NSP3, and SSA.

In some embodiments, the N. helminthoeca protein is P51. In some embodiments, the N. helminthoeca protein is NSP1. In some embodiments, the N. helminthoeca protein is NSP2. In some embodiments, the N. helminthoeca protein is NSP3. In some embodiments, the N. helminthoeca protein is SSA.

Further disclosed is a method of treating a Neorickettsia helminthoeca infection in a subject, comprising: assaying a sample from the subject for antibodies specific for a N. helminthoeca protein selected from the group consisting of P51, NSP1, NSP2, NSP3, and to SSA; and treating the subject for the Neorickettsia helminthoeca infection when antibodies specific for a N. helminthoeca protein selected from the group consisting of P51, NSP1, NSP2, NSP3, and SSA are present. In one embodiment, the subject is further treated with praziquantel, oral doxycycline, or intravenous oxytetracycline.

The details of one or more embodiments of the invention are set forth in the accompanying drawings and the description below. Other features, objects, and advantages of the invention will be apparent from the description and drawings, and from the claims.

### DESCRIPTION OF DRAWINGS

The accompanying figures, which are incorporated in and constitute a part of this specification, illustrate several aspects described below.

FIG. 1. Phylogenetic tree of the family Anaplasmataceae. 16S rRNA sequences of members of the family Anaplasmataceae were aligned using ClustalW, a phylogenetic tree was built using RAxML, and the tree was visualized with Dendroscope as described in the “Experimental procedures”. Gray box highlights Neorickettsia species.

GenBank Accession numbers and locus tag numbers for the 16S rRNA sequences are: N. helminthoeca Oregon, NZ₁₃ CP007481/NHE_RS00195; N. risticii Illinois, NC₁₃ 013009.1/NRI_RS00185; N. sennetsu Miyayama, NC_007798.1/NSE₁₃ RS00200; A. phagocytophilum HZ, NC_007797.1/APH₁₃ RS03965; A. marginale Florida, NC_012026.1/AMF_RS06130; E. chaffeensis Arkansas, NC_007799.1/ECH_RS03785; E. canis Jake, NC_007354.1/ECAJ_RS00995; E. ruminantium Welgevonden, NC_005295.2/ERUM_RS01035; E. muris AS145, NC_023063.1/MR76_RS00900; Ehrlichia sp. HF, NZ_CP007474.1/EHF_RS03625; Wolbachia pipientis wMel, NC_002978.6/WD_RS05540; Wolbachia endosymbiont of Brugia malayi, NC_006833.1/WBM_RS02885; Rickettsia rickettsii str. R, L36217; Neorickettsia Endobacterium of Fasciola hepatica, LNGI01000001/AS219_00180; Candidatus “Xenolissoclinum pacificiensis L6”, AXCJ01000001/P857_926.

FIG. 2. Circular representation of the genome of N. helminthoeca. From outside to inside, the first circle represents predicted protein coding sequences (ORFs) on the plus and minus strands, respectively. The second circle represents the unique ORFs of N. helminthoeca in the 3-way comparison with N. risticii and N. sennetsu. Colors indicate the functional role categories of ORFs—dark gray: hypothetical proteins or proteins with unknown functions; gold: amino acid and protein biosynthesis; sky blue: purines, pyrimidines, nucleosides, and nucleotides; cyan: fatty acid and phospholipid metabolism; light blue: biosynthesis of cofactors, prosthetic groups, and carriers; aquamarine: central intermediary metabolism; royal blue: energy metabolism; pink: transport and binding proteins; dark orange: DNA metabolism and transcription; pale green: protein fate; tomato: regulatory functions and signal transduction; peach puff: cell envelope; pink: cellular processes; maroon: mobile and extrachromosomal element functions. The third circle represent RNA genes, including tRNAs (blue), rRNAs (red), and ncRNAs (orange). The fourth circle represents GC skew values [(G−C)/(G+C)] with a windows size of 500 bp and a step size of 250 bp.

FIG. 3. Numbers of protein orthologs shared among Neorickettsia spp. A Venn diagram was constructed showing the comparison of conserved and unique genes between Neorickettsia spp. as determined by reciprocal BLASTP algorithm (E<e⁻¹⁰). Numbers within the intersections of different circles indicate ortholog clusters shared by 2 or 3 organisms. Species indicated in the diagram are abbreviated as follows: N. helminthoeca (NHO), N. sennetsu (NSE), N. risticii (NRI).

FIG. 4. Major metabolic pathways and secretion systems of N. helminthoeca. N. helminthoeca encodes pathways for aerobic respiration, including the tricarboxylic acid (TCA) cycle and the electron transport chain, but it is unable to use glucose, fructose, or fatty acids directly as a carbon or energy source. N. helminthoeca can synthesize very limited amino acids, but can synthesize most vitamins/cofactors, fatty acids, and certain phospholipids, and encodes complete pathways for de novo purine and pyrimidine biosynthesis. Putative transporters were analyzed by TransAAP (http://www.membranetransport.org/), and secretion systems were drawn as described in Results. Solid lines, pathways present; dashed lines, pathways absent; double lines, multiple steps involved. Graph was modified from KEGG pathways, J. C. Dunning Hotopp et al. (Dunning Hotopp et al., 2006), and J. J. Gillespie et al. (Gillespie et al., 2015).

FIG. 5. Genes involved in peptidoglycan biosynthesis in selected members of the family Anaplasmataceae. Biosynthesis pathways of peptidoglycan for N. helminthoeca, N. risticii, N. sennetsu, E. chaffeensis E. ruminatium, A. phagocytophilum, A. marginale, and Wolbachia wMel endosymbiont of Drosophila melanogaster were downloaded from KEGG database (http://www.genome.jp) and analyzed. N. helminthoeca, A. marginale, and Wolbachia wMel encode nearly all genes for peptidoglycan biosynthesis pathways (blue arrows), except that A. marginale and Wolbachia wMel lacks genes for the biosynthesis of D-Ala-D-Ala. In addition, all members in the family Anaplasmataceae encode terpenoid biosynthesis pathways like isopentenyl-, farnesyl-, and geranyl-diphosphate; however, only Neorickettsia and Wolbachia spp. encode undecaprenyl diphosphate (Und-PP) synthase (UppS) to produce Und-PP. N. helminthoeca encodes two PGPases (NHE_RS00895 and NHE_RS01205) that might produce Und-P from Und-PP. Genes present in N. risticii and N. sennetsu, red arrows; A. phagocytophilum, black arrows; E. chaffeensis and E. ruminantium, grey arrow. Dashed green lines, genes absent in all bacteria analyzed; dashed blue line, potential pathway present. Diagram was modified from KEGG pathways and J. J. Gillespie, et al. (Gillespie et al., 2010).

Abbreviations: GlcN, D-Glucosamine; GlcNAc, N-Acetyl-α-D-glucosamine; UDP-NAM, UDP-N-acetylmuramate; Undecaprenyl-PP (Und-PP), di-trans,poly-cis-undecaprenyl diphosphate; mDAP, meso-2,6-diaminopimelate; UDP-NAM-Tripeptide, UDP-NAM-L-Ala-D-Glu-mDAP, UDP-NAM-Pentapeptide, UDP-NAM-L-Ala-D-Glu-mDAP-D-Ala-D-Ala; Lipid I, Und-PP-NAM-L-Ala-D-Glu-mDAP-D-Ala-D-Ala; Lipid II, Und-PP-NAM-(GlcNAc)-L-Ala-D-Glu-mDAP-D-Ala-D-Ala; DAT, D-alanine transaminase; PGPase, phosphatidylglycerophosphatase.

FIGS. 6A-6C. Phylogenetic tree of putative outer membrane proteins in Neorickettsia spp. FIG. 6A shows the phylogenetic tree of putative outer membrane protein P51. FIG. 6B shows the phylogenetic tree of putative outer membrane proteins NSP 1/2/3. FIG. 6C shows the phylogenetic tree of putative outer membrane protein SSA. The amino acid sequences of putative OMPs (P51, NSPs, and SSAs) from N. helminthoeca, N. risticii and N. sennetsu were aligned with ClustalW, the phylogenetic tree was built using RAxML, and the tree was visualized with Dendroscope as described in the “Experimental to procedures”. N. helminthoeca encodes P51, NSP1/2/3, and one copy of SSA (closest to SSA3), while ssa2 gene of N. sennetsu is degenerated. For all three putative OMP groups (P51, NSPs, SSAs), N. helminthoeca OMPs forms a separate clade from those of N. risticii and N. sennetsu.

GenBank Accession numbers: 151 proteins—N. helminthoeca Oregon, WP_051579521; N. sennetsu Miyayama, WP_011451642; N. sennetsu strain 11908, AAL79561; N. sennetsu Nakazaki, AAR23990; N. risticii Illinois, WP_015816118; N. risticii strain 90-12, AAB46982; Neorickettsia sp. SF agent, AAR23988.

NSP Proteins: N. helminthoeca Oregon—NSP1, WP_038560103; NSP2, WP_038560106; NSP3, WP_038560109; N. sennetsu Miyayama—NSP1, WP_011452245; NSP2, WP_011452246; NSP3, WP_011452248; N. risticii Illinois—NSP1, WP_015816683; NSP2, WP_015816684; NSP3, WP_015816686.

SSA Proteins: N. helminthoeca Oregon—SSA, WP_038560160; N. sennetsu Miyayama—SSA1, WP_011452276; SSA3, WP_011452279; N. risticii Illinois—SSA1, WP_015816716; SSA2, WP_015816703; SSA3, WP_015816717.

FIGS. 7A-7F. Expression and immuno-reactivities of N. helminthoeca putative outer membrane proteins. P51, NSPs, and SSA proteins were cloned into pET33(+) expression vector and recombinant proteins were purified from transformed E. coli BL21(DE3) strain. The size and purity of these recombinant proteins were verified by GelCode blue protein stain (FIG. 6A). N. helminthoeca (70% infected DH82 cells) and N. risticii (90%-infected P388D1) from 2×T175 flasks were purified by sonication and filtration through 5-μm filters. ˜50 μg each of bacterial lysates from N. risticii (Nri) and N. helminthoeca (Nho), and ˜20 μg of purified recombinant outer membrane proteins of N. helminthoeca were subjected to Western blot analysis and probed with (FIG. 6B) Pony 19 sera against N. risticii from experimentally infected pony (1/400 dilution), (FIGS. 6C-6D) NH1 and NH3 sera against N. helminthoeca from the experimentally infected dogs, or (FIGS. 6E-6F) clinical dog sera from Southern California that were positive for N. helminthoeca-infection by PCR or IFA. Bands were visualized by ECL. The molecular size of the recombinant proteins are P51, 51.6 kDa; SSA, 33.7 kDa; NSPI, 27.7 kDa; NSP2, 32.2 kDa; NSP3, 23.7 kDa.

FIGS. 8A-8C. Synteny plots between Neorickettsia spp. The entire genomes of N. helminthoeca and N. risticii (FIG. 8B) or N. sennetsu (FIG. 8A) were aligned using MUMmer3 with default parameters. The entire genomes of N. risticii and N. sennetsu (FIG. 8C) were aligned using MUMmer3 with default parameters. Each axis represents the genomic coordinates for the respective organisms with red points reflecting matches on the forward strand and blue points reflecting matches on the reverse strand.

FIG. 9. Secondary Structure of N. helminthoeca P51 Protein. The two-dimensional structure of the N. helminthoeca P51 protein were predicted using PRED-TMBB analysis and image drawn by TMRPres2D (http://biophysics.biol.uoa.gr/PRED-TMBB/). The discrimination value for N. helminthoeca P51 is 2.949, which is below the threshold value of 2.965, suggesting that it is a β-barrel protein localized to the outer membrane with 18 transmembrane domains.

FIG. 10. Phylogenetic tree of VirB2 proteins in the family Anaplasmataceae and α-proteobacteria. Protein sequences of VirB2 from members of the family Anaplasmataceae and representative α-proteobacteria were aligned using the ClustalW method, and a phylogenetic tree was built using the MegAlign program of the Lasergene DNAstar package. Nho VirB2s, analyzed in this study from N. helminthoeca based on sequence homology to other Neorickettsia VirB2; Nse, N. sennetsu Miyayama; Nri, N. risticii Illinois; APH, A. phagocytophilum HZ; ECH, E. chaffeensis Arkansas; ATU16168, Agrobacierium tumefaciens C58 pilin subunit VirB2 (Accession No. NP_396488); RP192, Rickettsia prowazekii Madrid E VirB2 (Accession No. NP_359878); RC241, Rickettsia conorii Malish 7 VirB2 (Accession No. NP_359878); CC2417, Caulobacter crescentus CB15 VirB2 (Accession No. NP_421220).

FIG. 11. One-component regulatory systems of N. helminthoeca. The presence of genes encoding one-component regulatory systems in N. helminthoeca was predicted based on Microbial Signal Transduction Database (http://mistdb.com/). Domain architecture of each protein is predicted using the Pfam database. *Not identified in E. chaffeensis and A. phagocytophilum.

Domain abbreviations and functions: HTH, DNA-binding helix-turn helix domain; MerR, MerR family regulatory domain (DNA-binding, winged helix-turn-helix domain of about 70 residues present in the merR family of transcriptional regulators); Rrf2, Transcriptional regulator; Aminotran_5, Aminotransferase class V; EAL, EAL domain (diguanylate phosphodiesterase activity for degradation of a second messenger, cyclic di-GMP. Together with the GGDEF domain, EAL might be involved in regulating cell surface adhesiveness in bacteria); HD, HD domain (metal-dependent phosphohydrolases).

FIG. 12. Phylogenetic analysis of AnkA or Ank200 homologous proteins in the family Anaplasmataceae. Homologies of A. phagocytophilum HZ AnkA (GenBank accession No. WP_011450840) or E. chaffeensis Arkansas Ank200 (GenBank #WP_011452759) from representative members of the family Anaplasmataceae were first determined by Blast searches using E. chaffeensis Arkansas Ank200. Protein sequences were aligned using the ClustalW method, and a phylogenetic tree was built using the MegAlign program of the Lasergene DNAstar package. GenBank accession numbers for AnkA/Ank200 homologies are: N. helminthoeca Oregon, WP_038558671; N. sennetsu Miyayama, WP_011451432; N. risticii Illinois, WP 012779418; A. marginale St Maries, WP_011114402; E. canis Jake, WP_011304486; E. ruminantium Gardel, WP 011255523; Wolbachia pipientis wMel WP_010962493.

### DETAILED DESCRIPTION

Disclosed herein are isolated polypeptides comprising an amino acid sequence corresponding to Neorickettsia helminthoeca (NH) proteins, or functional derivatives thereof.

In some embodiments, the polypeptide comprises an NH P51 protein, or an immunogenic fragment thereof. Therefore in some embodiments, the polypeptide comprises the amino acid sequence SEQ ID NO:1.

Neorickettsia helminthoeca Oregon P51 Protein Sequence:

(SEQ ID NO: 1)
MICNIAKILFISTLLTSPVYASVENPSIGTRPPLEGKSCGCKKTCGCKKT
CGCSKNVHTGTSSGHNTINQPSFTIKGSSVFSFHYGKNEDFFELSKNLLK
IKNLPHSGTPTSASDVKPLYNVGISGEYDRPNKILSKSRISIEARRKMAD
FSYGVLLEPMFDMSKTVSTRNAYIFLEAPYGRFEMGQVNDSATSALKIDA
SSVAATGAGIRDLDWTEVANLEGRPEHAVFDTSTSSTQHKRHKNVTHPFL
VHPNYYVAYDAPIRANFTTTGLGAFKLAVSYTNRTADGIYRDILDFGCGY
TGIAKNLNYGVSITGQTSLEIPTGNLHHPLKRFEIGGMAEMYGIKLAGSF
GNSFLSGIKINKNMQLDLSKGIDDPKQFVSTNGQLTYMTLGTAFESGPMM
FSVNYMKSDNMLKKSDKSTLHVISIGTHYRLTGEAYELTPYVSGRYFVTS
EAGVPKGDNNKGYVISSGLKVSY.

In some embodiments, the polypeptide comprises an NH P51 functional derivative. In some embodiments, the polypeptide comprises an NH P51 variant. In some embodiments, the polypeptide comprises an NH P51 variant with an amino acid sequence which is at least 85% (for example, at least 85%, at least 90%, at least 95%) identical to SEQ ID NO:1.

In some embodiments, the polypeptide comprises an NH strain-specific antigen (SSA) protein, or an immunogenic fragment thereof. Therefore in some embodiments, the polypeptide comprises the amino acid sequence SEQ ID NO:2.

Neorickettsia helminthoeca Oregon SSA Protein Sequence:

(SEQ ID NO: 2)
MANGVTLFDILSNDTNFNTLTDSTVLDLLKHDTSSNTLKDTTAAEVLKNTT
AGDILKNSTAAEVLKNTTAGDILKNSTAAEVLKNTTAGDILKNSTAAEVLD
ANAKNVLENANAAAVLKDLGAAGTLKDATAAGALKDSEIQGLLKDKTAVDL
LKNASLCGVLKNNAERNLLKETDFQNLLKDQTAAGALKDSEIQGLLKDKTA
VDSLERAIVRDTLKCKDAAIVLQDEGFSALLRDNVNTEARNLLKETDFQNL
LKDQTAAGALKDSTIQGLLKDAAAIGALKQSGISELLKDTNAKRFLEDSAF
QASLKACESSSELQNRLKEITIPKK.

In some embodiments, the polypeptide comprises an NH SSA functional derivative. In some embodiments, the polypeptide comprises an NH SSA variant. In some embodiments, the polypeptide comprises an NH SSA variant with an amino acid sequence which is at least 85% (for example, at least 85%, at least 90%, at least 95%) identical to SEQ ID NO:2.

In some embodiments, the polypeptide comprises a Neorickettsia helminthoeca surface protein 1 (NSP1) protein, or an immunogenic fragment thereof. Therefore in some embodiments, the polypeptide comprises the amino acid sequence SEQ ID NO:3.

Neorickettsia helminthoeca Oregon NSP1 Protein Sequence:

(SEQ ID NO: 3)
MLGCRIAILLSLLLFLSPAEALFGINANTGFYISGGYGALMSGKAGVDNA
ATYANQAAQKFRSVSKDHLLHEDLKNFNVAAGFSILGFSLDVEGLYGYLE
SAKTSKNGTLKLKLPEKVGDQEFSYFLGFVNANLEFSGAALLNPYVGLGI
GTGTVTFAIENKDSDRRYGFPLATQIKAGLALDLGSYFFVSLKPYIGYRM
LMVSSTGVDTLSVVPTLIPTQNANPDAGIAGRIKEVVTAISDISHTSHNA
EIGIKIQLGI.

In some embodiments, the polypeptide comprises an NH NSP1 functional derivative. In some embodiments, the polypeptide comprises an NH NSP1 variant. In some embodiments, the polypeptide comprises an NH NSP1 variant with an amino acid sequence which is at least 85% (for example, at least 85%, at least 90%, at least 95%) identical to SEQ ID NO:3.

In some embodiments, the polypeptide comprises a Neorickettsia helminthoeca surface protein 2 (NSP2) protein, or an immunogenic fragment thereof. Therefore in some embodiments, the polypeptide comprises the amino acid sequence SEQ ID NO:4.

Neorickettsia helminthoeca Oregon NSP2 Protein Sequence:

(SEQ ID NO: 4)
MINSSFLRKALLLSCLFAMPLSGNSAAKVEEAANGVYGRIFQLSKVSGE
TNFMDTGRHYHHAVSEDVASLIKDSQHGPLLYHDGGVFGDYRPTHALNM
VGGGFALGYRTQNARFEFEGIINGEGKLSDSAESQFYGLAAVPAEVTKD
GKVNGQDHEGSGCKYLKGVKNVAVGPMNFSKFSYAATLFNIYQDITPGD
VMKLYVGGGVGISRVTYNLTSTQNLVSTPFVAQGKVGVTFDVGDLGSMG
MVPYLGYSALYFAEKEANSRVTGLTSHKMSKDKKGPCDKKDGIPGLEFA
PVAKHLLHNIEFGVTFSLDA.

In some embodiments, the polypeptide comprises an NH NSP2 functional derivative. In some embodiments, the polypeptide comprises an NH NSP2 variant. In some embodiments, the polypeptide comprises an NH NSP2 variant with an amino acid sequence which is at least 85% (for example, at least 85%, at least 90%, at least 95%) identical to SEQ ID NO:4.

In some embodiments, the polypeptide comprises a Neorickettsia helminthoeca surface protein 3 (NSP3) protein, or an immunogenic fragment thereof. Therefore in some embodiments, the polypeptide comprises the amino acid sequence SEQ ID NO:5.

Neorickettsia helminthoeca Oregon NSP3 Protein Sequence:

(SEQ ID NO: 5)
MINKKFLISVALAGVASTSDAQDALEDADIFYAKVGYNATKMQPVEWTKA
RVSGDTSKFKPEYESSFIGGSAALGYYFGGMRVELEGSMYNVDSKKGSKI
PETKQPDAPAIKYGGACFMGGMLSVNYDVALTDYISPYFGVGFGLSRVSL
KLDDDALSTAYHMSSQLKGGVSITGLAAVVPYAGYKFTYMNDKGYSKVAL
ANSTELAPQLSHMVHNFEAGLMLPMAN.

In some embodiments, the polypeptide comprises an NH NSP3 functional derivative. In some embodiments, the polypeptide comprises an NH NSP3 variant. In some embodiments, the polypeptide comprises an NH NSP3 variant with an amino acid sequence which is at least 85% (for example, at least 85%, at least 90%, at least 95%) identical to SEQ ID NO:5.

Also provided herein are functional derivatives of the NH proteins enumerated above. A “functional derivative” of an NH protein or peptide sequence is a molecule that possesses immunoreactivity to NH antibodies that is substantially similar to that of the corresponding NH protein or peptide, i.e. an “immunoreactive” functional derivative is a polypeptide that has a specific binding affinity for anti-N. helminthoeca antibodies.

The functional derivatives of an NH protein can be identified using any of a variety of routine assays for detecting peptide antigen-antibody complexes, the presence of which is an indicator of selective binding. Such assays include, without limitation, enzyme-linked immunosorbent assays (ELISA), radioimmunoassays, western blotting, enzyme immunoassays, fluorescence immunoassays, luminescent immunoassays and the like. Methods for detecting a complex between a peptide and an antibody, and thereby determining if the peptide is an “immunoreactive functional derivative” are well known to those skilled in the art and are described, for example, in ANTIBODIES: A LABORATORY MANUAL (Edward Harlow & David Lane, eds., Cold Spring Harbor Laboratory Press, 2.sup.nd ed. 1998a); and USING ANTIBODIES: A LABORATORY MANUAL: PORTABLE PROTOCOL No. I (Edward Harlow & David Lane, Cold Spring Harbor Laboratory Press, 1998b), which are hereby incorporated by reference in their entirety.

Thus, the terms “functional derivative” and “immunoreactive functional derivative” are used interchangeably and refer to peptides and proteins that can function in substantially the same manner as the NH proteins or peptides disclosed herein, and can be substituted for the N. helminthoeca proteins or peptides in the disclosed compositions and methods.

A “functional derivative” of a protein or peptide can contain post-translational modifications such as covalently linked carbohydrate, depending on the necessity of such modifications for the performance of a specific function. The term “functional derivative” is intended to include the immunoreactive “variants” and “fragments” of the NH proteins.

A “variant” of an NH protein refers to a molecule substantially similar in structure and immunoreactivity to the NH protein. Thus, provided that two molecules possess a common immunoactivity and can substitute for each other, they are considered “variants” as that term is used herein even if the composition or secondary, tertiary, or quaternary structure of one of the molecules is not identical to that found in the other, or if the amino acid or nucleotide sequence is not identical. Thus, in one embodiment, a variant refers to a protein whose amino acid sequence is similar to the amino acid sequences of a mature NH protein, hereinafter referred to as the reference amino acid sequence, but does not have 100% identity with the respective reference sequence. The variant protein has an altered sequence in which one or more of the amino acids in the reference sequence is deleted or substituted, or one or more amino acids are inserted into the sequence of the reference amino acid sequence. As a result of the alterations, the variant protein has an amino acid sequence which is at least 85%, 86%, 88%, 89%, 90%, 91%, 92%, 93%, 94%, 95%, 96%, 97%, 98%, or 99% identical to the reference sequence. For example, variant sequences which are at least 95% identical have no more than 5 alterations, i.e. any combination of deletions, insertions or substitutions, per 100 amino acids of the reference sequence. Percent identity is determined by comparing the amino acid sequence of the variant with the reference sequence using any available sequence alignment program. An example includes the MEGALIGN project in the DNA STAR program. Sequences are aligned for identity calculations using the method of the software basic local alignment search tool in the BLAST network service (the National Center for Biotechnology Information, Bethesda, Md.) which employs the method of Altschul, S. F., Gish, W., Miller, W., Myers, E. W. & Lipman, D. J. (1990) J. Mol. Biol. 215, 403-410. Identities are calculated by the Align program (DNAstar, Inc.) In all cases, internal gaps and amino acid insertions in the candidate sequence as aligned are not ignored when making the identity calculation.

Variants of the NH proteins can include nonconservative as well as conservative amino acid substitutions. A conservative substitution is one in which the substituted amino acid has similar structural or chemical properties with the corresponding amino acid in the reference sequence. By way of example, conservative amino acid substitutions involve substitution of one aliphatic or hydrophobic amino acids, e.g. alanine, valine, leucine and isoleucine, with another; substitution of one hydroxyl-containing amino acid, e.g. serine and threonine, with another; substitution of one acidic residue, e.g. glutamic acid or aspartic acid, with another; replacement of one amide-containing residue, e.g. asparagine and glutamine, with another; replacement of one aromatic residue, e.g. phenylalanine and tyrosine, with another; replacement of one basic residue, e.g. lysine, arginine and histidine, with another; and replacement of one small amino acid, e.g., alanine, serine, threonine, methionine, and glycine, with another.

The alterations are designed not to abolish the immunoreactivity of the variant NH protein with antibodies that bind to the reference protein. Guidance in determining which amino acid residues may be substituted, inserted or deleted without abolishing such immunoreactivity of the variant protein are found using computer programs well known in the art, for example, DNASTAR software.

Preparation of an NH protein variant in accordance herewith can be achieved by site-specific mutagenesis of DNA that encodes an earlier prepared variant or a nonvariant version of the protein. Site-specific mutagenesis allows the production of NH protein variants through the use of specific oligonucleotide sequences that encode the DNA sequence of the desired mutation. In general, the technique of site-specific mutagenesis is well known in the art, as exemplified by publications such as Adelman et al., DNA 2:183 (1983) and Ausubel et al. “Current Protocols in Molecular Biology”, J. Wiley & Sons, NY, N.Y., 1996. As will be appreciated, the site-specific mutagenesis technique can employ a phage vector that exists in both a single-stranded and double-stranded form. Typical vectors useful in site-directed mutagenesis include vectors such as the M13 phage, for example, as disclosed by Messing et al., Third Cleveland Symposium on Macromolecules and Recombinant DNA, Editor A. Walton, Elsevier, Amsterdam (1981). These phage are readily commercially available and their use is generally well known to those skilled in the art. Alternatively, plasmid vectors that contain a single-stranded phage origin of replication (Vieira et al., Meth. Enzymol. 153:3 (1987)) can be employed to obtain single-stranded DNA.

In general, site-directed mutagenesis in accordance herewith is performed by first obtaining a single-stranded vector that includes within its sequence a DNA sequence that encodes the relevant protein. An oligonucleotide primer bearing the desired mutated sequence is prepared, generally synthetically, for example, by the method of Crea et al., Proc. Natl. Acad. Sci. (USA) 75:5765 (1978). This primer is then annealed with the single-stranded protein-sequence-containing vector, and subjected to DNA-polymerizing enzymes such as E. coli polymerase I Klenow fragment, to complete the synthesis of the mutation-bearing strand. Thus, a mutated sequence and the second strand bears the desired mutation. This heteroduplex vector is then used to transform appropriate cells and clones are selected that include recombinant vectors bearing the mutated sequence arrangement. After such a clone is selected, the mutated protein region can be removed and placed in an appropriate vector for protein production, generally an expression vector of the type that can be employed for transformation of an appropriate host.

Some deletions and insertions, and substitutions are not expected to produce radical changes in the characteristics of NH proteins. However, when it is difficult to predict the exact effect of the substitution, deletion, or insertion in advance of doing so, one skilled in the art will appreciate that the effect will be evaluated by routine screening assays. For example, a variant typically is made by site-specific mutagenesis of the native encoding nucleic acid, expression of the variant nucleic acid in recombinant cell culture, and, optionally, purification from the cell culture, for example, by immunoaffinity adsorption to on a column (to absorb the variant by binding it to at least one remaining immune epitope). The activity of the cell lysate or purified variant is then screened in a suitable screening assay for the desired characteristic. For example, a change in the immunological character of the molecule, such as affinity for a given antibody, is measured by a competitive type immunoassay. Changes in immunomodulation activity are measured by the appropriate assay. Modifications of such protein properties as redox or thermal stability, hydrophobicity, susceptibility to proteolytic degradation or the tendency to aggregate with carriers or into multimers are assayed by methods well known to the ordinarily skilled artisan.

A “fragment” is an immunoreactive fragment of an NH protein that has a length of from about 6 amino acids to less than the full length NH protein and includes a sequence that contains at least 6 consecutive amino acids of a sequence of the NH protein. These fragments are collectively referred to herein as “NH peptides.” In some embodiments, the fragment has at least 6, 7, 8, 9, 10, 11, 12, 13, 14, 15, 16, 17, 18, 19, 20, 25, 30, 35, 40, 45, 50, 60, 70, 80, 90, or 100 consecutive amino acids of an NH protein sequence. The fragment can have a length of at most, e.g., 6, 7, 8, 9, 10, 11, 12, 13, 14, 15, 16, 17, 18, 19, 20, 25, 30, 35, 40, 45, 50, 60, 70, 80, 90, 100, 120, 140, 160, 180, 200, 220, 240, 260, 280, or 300 amino acids. In some embodiments, an immunoreactive fragment has from six to sixty amino acids, from six to fifty amino acids, from ten to fifty amino acids, from six to twenty amino acids, from eight to twenty amino acids, from ten to twenty amino acids, from twelve to twenty amino acids or from twelve to seventeen amino acids.

In some embodiments, the immunoreactive peptides are from six (6) amino acids up to less than the full length NH protein, and are antigenic, i.e. are recognized by mammalian immune systems effectively. For this purpose, the peptides comprise segments that are bacterial surface exposed, rather than bacterial cytoplasmic side-exposed or embedded within the lipid bilayer membrane. Such surface exposed regions of NH proteins can be identified using computer programs using algorithms that can predict the three dimensional structure of the NH proteins based on the hydrophobicity/hydrophilicity of the amino acid regions and the repeated β sheet model.

Also provided herein are fusion proteins in which a tag or one or more amino acids from a heterologous protein are added to the amino or carboxy terminus of the amino acid sequence of an NH protein or a functional derivative thereof. At least one of the proteins or peptides can be in a multimeric form. As used herein, the term “heterologous protein” means a protein derived from a source other than the N. helminthoeca gene, operationally linked to a N. helminthoeca protein or a functional derivative thereof, as disclosed in the present specification, to form a chimeric or fusion N. helminthoeca protein or peptide. Typically, such additions are made to stabilize the resulting fusion protein or to simplify purification of an expressed recombinant form of the corresponding NH protein, variant, or peptide. Such tags are known in the art. Representative examples of such tags include sequences which encode a series of histidine residues, the Herpes simplex glycoprotein D, or glutathione S-transferase. Such a chimeric or fusion protein can have a variety of lengths including, but not limited to, a length of at most 100 residues, at most 200 residues, at most 300 residues, at most 400 residues, at most 500 residues, at most 800 residues or at most 1000 residues. Non-limiting examples of chimeric N. helminthoeca proteins include fusions of N. helminthoeca protiens, or variants, or peptides: with immunogenic polypeptides, such as flagellin and cholera enterotoxin; with immunomodulatory polypeptides, such as IL-2 and B7-1; with tolerogenic polypeptides; with another N. helminthoeca protein, or variant, or peptide; and with synthetic sequences. Other examples include linking the NH protein, or variant or peptide with an indicator reagent, an amino acid spacer, an amino acid linker, a signal sequence, a stop transfer sequence, a transmembrane domain, a protein purification ligand or a combination of thereof. The fusion proteins can have similar or substantially similar immunoreactivity to NH antibodies as the NH proteins from which they derive.

The disclosed NH polypeptides can be used in a variety of procedures and methods, such as for the generation of antibodies, immunogenic compositions and vaccines; for use in identifying pharmaceutical compositions; for studying DNA/protein interaction; as well as for diagnostic and screening methods.

Also provided are compositions of matter comprising one or more NH proteins, their functional derivatives and/or NH fusion proteins. The isolated or purified polypeptide in such compositions can be in a multimeric form and can further include a carrier. The purified polypeptide can be linked to an indicator reagent, an amino acid spacer, an amino acid linker, a signal sequence, a stop transfer sequence, a transmembrane domain, a protein purification ligand, or a combination of these. Alternatively, one or more NH proteins or peptides may be linked together.

Also disclosed are polynucleotides encoding an NH protein, or variant thereof, disclosed herein.

In some embodiments, the polynucleotide encodes an NH P51 protein, or an immunogenic fragment thereof. Therefore in some embodiments, the polynucleotide comprises the nucleic acid sequence SEQ ID NO:6.

Neorickettsia helminthoeca Oregon P51 Gene Sequence:

(SEQ ID NO: 6)
ATGATATGCAACATCGCTAAAATTCTATTCATTTCTACATTGCTCACAAG
TCCTGTATACGCTTCTGTAGAGAACCCATCAATTGGAACAAGACCACCTC
TAGAAGGGAAAAGCTGTGGATGTAAGAAAACTTGTGGATGTAAGAAAACT
TGTGGATGTAGCAAAAATGTCCATACAGGTACTTCTTCTGGTCATAATAC
AATAAATCAACCATCTTTCACAATAAAGGGAAGTAGTGTTTTCTCGTTCC
ACTATGGGAAGAATGAAGATTTTTTCGAACTTAGTAAAAACCTATTGAAA
ATCAAGAACCTTCCGCACAGTGGAACACCAACTAGCGCTAGTGATGTTAA
ACCCCTATATAACGTAGGTATCTCAGGTGAGTATGACCGTCCAAATAAAA
TCCTCAGCAAAAGTAGGATATCAATCGAGGCAAGACGTAAAATGGCAGAC
TTCTCTTATGGAGTTCTGCTAGAACCGATGTTCGATATGAGTAAAACAGT
CAGCACCAGGAACGCATATATCTTCCTTGAAGCACCGTATGGAAGATTTG
AGATGGGCCAAGTTAATGATAGCGCAACCTCAGCACTGAAAATTGATGCA
TCGTCAGTTGCAGCTACCGGCGCAGGAATCAGAGATTTGGATTGGACTGA
AGTCGCAAACCTTGAAGGAAGGCCTGAACACGCTGTATTTGATACCAGCA
CTAGTAGCACACAGCATAAAAGACATAAAAATGTAACTCACCCGTTCTTG
GTCCACCCGAATTATTATGTAGCATATGATGCTCCAATCAGAGCGAATTT
CACCACTACTGGACTCGGCGCATTCAAATTAGCAGTGAGTTACACAAACA
GAACTGCTGATGGAATATATCGCGATATTTTGGATTTCGGTTGTGGATAT
ACCGGAATTGCAAAGAATCTGAACTATGGTGTTTCCATCACTGGGCAAAC
CAGCCTCATAGAGCCAACTGGAAATCTGCACCATCCTCTAAAGAGATTCG
AGATTGGCGGAATGGCAGAGATGTATGGTATCAAGCTTGCAGGATCATTT
GGCAATFCTTTCCTTTCTGGAATTAAAATAAATAAAAACATGCAACTTGA
TCTCTCAAAGGGTATAGATGATCCAAAGCAATTTGTCAGTACAAACGGTC
AACTTACCTATATGACATTAGGTACAGCATTCGAAAGTGGCCCAATGATG
TTCAGTGTCAACTACATGAAGAGCGATAATATGTTGAAAAAATCCGACAA
AAGTACATTCGCATGTTATTTCTATTGGAACACACTACCGCTTAACAGGA
GAAGCGCATGAACTCACTCCTTATGTGAGTGGAAGATATTTTGTCACCTC
AGAAGCTGGTGTACCAAAAGGTGATAATAACAAAGGTTATGTAATTTCTT
CAGGTCTCAAAGTATCATATTGA.

In some embodiments, the polynucleotide encodes an NH P51 functional derivative. In some embodiments, the polynucleotide encodes an NH P51 variant. In some embodiments, the polynucleotide encodes an NH P51 variant with a nucleic acid sequence which is at least 85% (for example, at least 85%, at least 90%, at least 95%) identical to SEQ ID NO:6.

In some embodiments, the polynucleotide encodes an NH SSA protein, or an immunogenic fragment thereof. Therefore in some embodiments, the polynucleotide comprises the nucleic acid sequence SEQ D NO:7.

Neorickettsia helminthoeca Oregon SSA Gene Sequence:

(SEQ ID NO: 7)
ATGGCAAACGGTGTCACACTATTTGATATTTTGTCAAATGACACTAATTT
TAACACCTTAACCGATAGTACGGTCCTTGATCTGCTTAAGCATGATACCT
CAAGTAATACATTAAAAGATACAACCGCAGCTGAGGTATTAAAAAATACA
ACTGCTGGAGATATATTAAAGAATTCAACCGCAGCTGAGGTATTAAAAAA
TACAACTGCTGGAGATATATTAAAGAATTCAACCGCAGCTGAGGTATTAA
AAAATACAACTGCTGGAGATATATTAAAGAATTCAACCGCAGCTGAGGTA
CTAAAAGATGCAAATGCAAAAAATGTACTGGAAAACGCAAATGCAGCTGC
GGTATTAAAAGATTTAGGCGCGGCGGGGACCCTAAAAGATGCAACAGCAG
CAGGTGCCTTAAAAGATTTTCAGAAATTCAGGGCTTGTTAAAGGATAAGA
CCGCGGTAGACCTTTTAAAGAATGCAAGTCTCTGCGGAGTGTTAAAAAAC
AATGCAGAAGCTAGAAACCTTTTGATTAGAGACAGACTTCCAGAATCTAT
TAAAGGATCAGACAGCAGCAGGTGCCTTAAAAGATTCAGAAATTCAGGGC
TTGTTAAAGGATAAGACCGCGGTAGACAGCTTAGAAAGGGCGATTGTTCG
GGATACGCTAAAGTGCAAAGACGCAGCAATCGTTTTGCAAGATGAAGGAT
TCAGCGCTCTATTACGAGATAATGTCAATACAGAAGCTAGAAACCTTTTG
AAAGAGACAGACTTCCAGAATCTATTAAAGGATCAGACAGCAGCAGGTGC
CTTAAAAGATTCAACAATTCAGGGCCTATTAAAGGATGCAGCTGCGATAG
GGGCTTTAAAACAATCGGGTATTTCTGAGTTGTTGAAGGATACTAATGCC
AAGAGATTCTTAGAGGATAGTGCCTTCCAAGCCTCATTAAAGGCTTGTGA
GAGCTCAAGTGAGCTACAGAATAGACTTAAAGAGATAACTATCCCCAAAA
AATAA.

In some embodiments, the polynucleotide encodes an NH SSA functional derivative. In some embodiments, the polynucleotide encodes an NH SSA variant. In some embodiments, the polynucleotide encodes an NH SSA variant with a nucleic acid sequence which is at least 85% (for example, at least 85%, at least 90%, at least 95%) identical to SEQ ID NO:7.

In some embodiments, the polynucleotide encodes an NH NSP1 protein, or an immunogenic fragment thereof. Therefore in some embodiments, the polynucleotide comprises the nucleic acid sequence SEQ ID NO:8.

Neorickettsia helminthoeca Oregon NSP1 Gene Sequence:

(SEQ ID NO: 8)
ATGCTCGGATGTCGTATCGCTATTTTGCTGTCTCTGCTACTCTTTTTTGA
GTCCTGCTGAGGCGCTTTTCGGAATAAACGCGAACACCGGGTTTTACATC
AGTGGTGGATATGGCGCTTTGATGTCTGGCAAGGCGGGTGTTGATAATGC
TTGCCACTTATGCAAATCAAGCAGCTCAGAAATTTAGAAGTGTGAGCAAG
GATCATCTGCTTCACGAGGATCTGAAGAACTTCAATGTTGGCAGCTGGGT
TTTCAATTTTTAGGATTcTCATTGGACGTTGAAGGTCTCTATGCATATCT
TGAATCTGCGAAAACAAGTAAAAACGGTACCCTCAAACTCAAATTGCCAG
AAAAAGTTGGTGATCAGGAATTTTCCTATTTTCTTGGCTTTGTTAACGCG
AATCTGGAATTCTCAGGAGCGGCGTTATTGAATCCCTACGTTGGATTAGG
TATCGGCACCGGGACTGTCACATTCGCTATTGAGAATAAGGATTCGGATA
GGAGATACGGATTTCCTCTGGCGACGCAGATAAAAGCTGGCTTAGCGCTT
GATCTAGGATCCTATTTCTTTGTCTCATTGAAGCCGTATATTGGTTATCG
GATGCTGATGGTCTCTAGTACGGGAGTCGATACACTTTCCGTTGTCCCTA
CACTCTTTCCGACGCAGAATGCAAATCCTGATGCAGGAATAGCTGGTAGG
ATCAAGGAAGTTGTCACTGCAATCAGTGATATTAGTCACACCTCGCATAT
TGCTGAGATTGGAATCAAGATCCAGCTTGGAATATAA.

In some embodiments, the polynucleotide encodes an NH NSP1 functional derivative. In some embodiments, the polynucleotide encodes an NH NSP1 variant. In some embodiments, the polynucleotide encodes an NH NSP1 variant with a nucleic acid sequence which is at least 85% (for example, at least 85%, at least 90%, at least 95%) identical to SEQ ID NO:8.

In some embodiments, the polynucleotide encodes an NH NSP2 protein, or an immunogenic fragment thereof. Therefore in some embodiments, the polynucleotide comprises the nucleic acid sequence SEQ ID NO:9.

Neorickettsia helminthoeca Oregon NSP2 Gene Sequence:

(SEQ ID NO: 9)
ATGATTAATAGTAGTTTTTTGAGAAAGGCATTACTCCTCTCCTGTTTGTT
TGCGATGCCGCTGAGTGGCAACAGTGCTGCCAAAGTAGAAGAAGCGGCGA
ATGCAGGTGTTTATGGTAGAATTTTCCAGCTAAGCAAGGTTAGCGGCGAA
ACTAATTTTATGGACACTGGGCGCCATTACCACCATGCAGTTAGTGAAGA
TGTTGCTAGCCTGATTAAAGATTCACAGCATGGCCCATTATTATACCACG
ATGGTGGCGTTTTTGGAGACTACAGGCCTACACATGCACTTAACATGGTA
GGTGGTGGTTTTGCACTTGGATACCGCACCCAAAACGCAAGGTTTGAGTT
TGAAGGGATAATAAACGGCGAAGGTAAACTAAGTGACAGCGCTGAATCAC
AGTTTTATGGTCTTGCTGCTGTACCAGCTGAGGTAACCAAAGATGGTAAA
GTAAATGGACAGGACCATGAGGGATCAGGATGTAAGTACCTCAAAGGCGT
GAAGAATGTGGCGGTTGGCCCAATGAACTTTAGTAAGTTCTCTTATGCGG
CTACCCTGTTTAATATCTATCAGGATATTCCAACTGGAGATGTAATGAAA
TTGTATGTAGGCGGTGGTGTCGGAATAAGCCGTGTTACTTACAACTTGAC
AAGTACTCAAAACCTTGTTAGCACTCCATTTGTTGCGCAGGGTAAGGTCG
GTGTAACCTTTGATGTCGGCGATCTAGGAAGTATGGGCATGGTACCATAT
CTTGGCTACTCAGCGCTCTACTTCGCTGAAAAAGAAGCTAATAGTCGCGT
GACAGGTCTAACTAGCCACAAAATGAGCAAGGATAAAAAGGGCCCTTGCG
ACAAGAAGATGGTATCCCAGGACTTGAGTTTGCGCCTGTGGCAAAACACT
TGCTACATAACATTGAGTTTGGGGTTACTTTTTCACTTGACGCCTGA.

In some embodiments, the polynucleotide encodes an NH NSP2 functional derivative. In some embodiments, the polynucleotide encodes an NH NSP2 variant. In some embodiments, the polynucleotide encodes an NH NSP2 variant with a nucleic acid sequence which is at least 85% (for example, at least 85%, at least 90%, at least 95%) identical to SEQ ID NO:9.

In some embodiments, the polynucleotide encodes an NH NSP3 protein, or an immunogenic fragment thereof. Therefore in some embodiments, the polynucleotide comprises the nucleic acid sequence SEQ ID NO:10.

Neorickettsia helminthoeca Oregon NSP3 Gene Sequence:

(SEQ ID NO: 10)
ATGATAAATAAAAAGTTCCTAATAAGCGTGGCTCTTGCAGGTGTTCTTTG
CCTTGCATCTACCTCAGATGCGCAAGATGCCCTAGAGGATGCAGATATTT
TCTATGCCAAAGTTGGGTATAACGCTACCAAAATGCAGCCGGTGGAGTGG
ACTAAGGCCCGCGTATCGGGTGATACTAGTAAATTCAAGCCAGAGTATGA
AAGTAGTTTCATTGGCGGTAGTGCTGCTCTCGGATATTACTTCGGTGGCA
TGAGAGTCGAACTGGAAGGCAGCATGTATAATGTTGATTCTAAAAAAGGT
TCTAAAATACCTGAAACTAAGCAGCCCGATGCACCTGCTATAAAGTATGG
TGGCGCTTGTTTTATGGGTGGCATGCTTTCAGTAAACTACGATGTGGCTC
TAACTGATTATATCAGCCCGTACTTTGGAGTAGGTTTCGGTCTAAGCAGA
GTATCCCTAAAGCTTGATGATGATGCATTGTCTACTGCGTATCATATGTC
ATCCCAATTGAAAGGTGGTGTAAGCATCACTGGGCTCGCTGCTGTGGTCC
CTTATGCTGGATATAAGTTCACATATATGAATGACAAAGGTTATTCAAAA
GTAGCTCTTGCTAATAGTACTGAGCTTGCTCCGCAACTTTCTCATATGGT
GCACAACTTTGAGGCTGGTCTAATGCTACCTATGAATGCGTAA.

In some embodiments, the polynucleotide encodes an NH NSP3 functional derivative. In some embodiments, the polynucleotide encodes an NH NSP3 variant. In some embodiments, the polynucleotide encodes an NH NSP3 variant with a nucleic acid sequence which is at least 85% (for example, at least 85%, at least 90%, at least 95%) identical to SEQ ID NO:10.

Also disclosed are polynucleotides complementary to the disclosed nucleic acid sequences. Also disclosed are polynucleotides that can hybridize to a nucleic acid sequence disclosed herein under stringent hybridization conditions, or highly stringent hybridization conditions. It is understood that the polynucleotides encoding the NH polypeptides can have a different sequence than the nucleotide sequences disclosed herein due to the degeneracy of the genetic code. Thus, also included are the functional equivalents of the herein-described isolated polynucleotides and derivatives thereof. For example, the nucleic acid sequences can be altered by substitutions, additions or deletions that provide for functionally equivalent molecules. In addition, the polynucleotide can comprise a nucleotide sequence which results from the addition, deletion or substitution of at least one nucleotide to the 5′-end and/or the 3′-end of the disclosed nucleic acid segments, or a derivative thereof. Any polynucleotide can be used in this regard, provided that its addition, deletion or substitution does not substantially alter the amino acid sequence of the NH protein, or functional derivatives or fusion proteins thereof, encoded by the polynucleotide sequence. Moreover, the polynucleotide of the present invention can, as necessary, have restriction endonuclease recognition sites added to its 5′-end and/or 3′-end.

Further, it is possible to delete codons or to substitute one or more codons by codons other than degenerate codons to produce a structurally modified polypeptide, but one which has substantially the same utility or activity of the polypeptide produced by the unmodified nucleic acid molecule. As recognized in the art, the two polypeptides are functionally equivalent, as are the two nucleic acid molecules which give rise to their production, even though the differences between the nucleic acid molecules are not related to degeneracy of the genetic code.

The NH polynucleotides described herein are also useful for designing hybridization probes for isolating and identifying cDNA clones and genomic clones encoding the NH proteins, peptides or allelic forms thereof. Such hybridization techniques are known to those of skill in the art.

Therefore, in another embodiment, a nucleic acid probe is provided for the specific detection of the presence of one or more NH polynucleotides in a sample comprising the above-described isolated polynucleotides or at least a fragment thereof, which binds under stringent conditions, or highly stringent conditions, to NH polynucleotides.

The term “stringent conditions” as used herein is the binding which occurs within a range from about Tm 5° C. (5° C. below the melting temperature Tm of the probe) to about 20° C. to 25° C. below Tm. The term “highly stringent hybridization conditions” as used herein refers to conditions of: at least about 6×SSC and 1% SDS at 65° C., with a first wash for 10 minutes at about 42° C. with about 20% (v/v) formamide in 0.1×SSC, and with a subsequent wash with 0.2×SSC and 0.1% SDS at 65° C.

In some embodiments, the isolated nucleic acid probe consisting of 10 to 1000 nucleotides (for example: 10 to 500, 10 to 250, 10 to 100, 10 to 50, 10 to 35, 20 to 1000, 20 to 500, 20 to 250, 20 to 100, 20 to 50, or 20 to 35, etc.) which hybridizes preferentially to RNA or DNA of NH but not to RNA or DNA of non-NH organisms, wherein said nucleic acid probe is or is complementary to a nucleotide sequence consisting of at least 10 consecutive nucleotides, or 15, 20, 25, 30, 50, 100, 250, 500, 600, 700, 800, or 900 consecutive nucleotides, or along the entire length, of one or more of the NH polynucleotides described above.

Such hybridization probes can have a sequence which is at least 90%, 95%, 98%, 99% or 100% complementary with a sequence contained within the sense strand of a DNA molecule which encodes each of the NH proteins or with a sequence contained within its corresponding antisense strand. Such hybridization probes bind to the sense or antisense strand under stringent, or highly stringent, conditions.

The hybridization probes can be labeled by standard labeling techniques such as with a radiolabel, enzyme label, fluorescent label, biotin-avidin label, chemiluminescence, and the like. After hybridization, the probes can be visualized using known methods.

In some embodiments, a nucleic acid probe is immobilized on a solid support. Examples of such solid supports include, but are not limited to, plastics such as polycarbonate, complex carbohydrates such as agarose and sepharose, and acrylic resins, such as polyacrylamide and latex beads. Techniques for coupling nucleic acid probes to such solid supports are well known in the art.

NH polynucleotides disclosed herein are also useful for designing primers for polymerase chain reaction (PCR), a technique useful for obtaining large quantities of cDNA molecules that encode the NH polypeptides. PCR primers can also be used for diagnostic purposes. Thus, also included are oligonucleotides that are used as primers in polymerase chain reaction (PCR) technologies to amplify transcripts of the genes which encode the NH polypeptides, or portions of such transcripts. In some examples, the primers comprise a minimum of about 12 to 15 nucleotides and a maximum of about 30 to 35 nucleotides. The primers can have a G+C content of 40% or greater. Such oligonucleotides are at least 98% complementary with a portion of the DNA strand, i.e., the sense strand, which encodes the NH protein, or a portion of its corresponding antisense strand. In some embodiments, the primer has at least 99% complementarity, or 100% complementarity, with such sense strand or its corresponding antisense strand. Primers which have 100% complementarity with the antisense strand of a double-stranded DNA molecule encoding an NH protein have a sequence which is identical to a sequence contained within the sense strand.

One skilled in the art can readily design such probes and primers based on the sequences disclosed herein using methods of computer alignment and sequence analysis known in the art (see, for example, Molecular Cloning: A Laboratory Manual, second edition, edited by Sambrook, Fritsch, & Maniatis, Cold Spring Harbor Laboratory, 1989).

The primers herein are selected to be “substantially” complementary to different strands of a particular target DNA sequence. This means that the primers must be sufficiently complementary to hybridize with their respective strands. Therefore, the primer sequence need not reflect the exact sequence of the template. For example, a non-complementary nucleotide fragment may be attached to the 5′ end of the primer, with the remainder of the primer sequence being complementary to the strand. Alternatively, non-complementary bases or longer sequences can be interspersed into the primer, provided that the primer sequence has sufficient complementary with the sequence or hybridize therewith and thereby form the template for the synthesis of the extension product.

Also disclosed are methods for diagnosing a canine subject with Neorickettsia helminthoeca infection using the disclosed polypeptides to detect antibodies specific for Neorickettsia helminthoeca in a sample from the subject. For example, the sample can be a blood, serum, or plasma sample containing antibodies. Immunodetection methods can be used to assay for the presence of antibodies that specifically bind an NH protein or peptide disclosed herein.

The method can involve contacting the sample with one or more Neorickettsia helminthoeca polypeptides, as described herein, under conditions that allow polypeptide/antibody complexes to form; and assaying for the formation of a complex between antibodies in the test sample and the one or NH polypeptides. Accordingly, detecting the formation of such a complex is an indication that antibodies specific for Neorickettsia helminthoeca are present in the test sample.

The steps of various useful immunodetection methods have been described in the scientific literature, such as, e.g., Maggio et al., Enzyme-Immunoassay, (1987) and Nakamura, et al., Enzyme Immunoassays: Heterogeneous and Homogeneous Systems, Handbook of Experimental Immunology, Vol. 1: Immunochemistry, 27.1-27.20 (1986), each of which is incorporated herein by reference in its entirety and specifically for its teaching regarding immunodetection methods. Immunoassays, in their most simple and direct sense, are binding assays involving binding between antibodies and antigen. Many types and formats of immunoassays are known and all are suitable for detecting the disclosed biomarkers. Examples of immunoassays are enzyme linked immunosorbent assays (ELISAs), radioimmunoassays (RIA), radioimmune precipitation assays (RIPA), immunobead capture assays, Western blotting, dot blotting, gel-shift assays, Flow cytometry, protein arrays, multiplexed bead arrays, magnetic capture, in vivo imaging, fluorescence resonance energy transfer (FRET), and fluorescence recovery/localization after photobleaching (FRAP/FLAP).

Also disclosed are immunogenic compositions comprising one or more of the disclosed Neorickettsia helminthoeca proteins, or immunogenic fragments and variants thereof, or a fusion protein containing same, collectively referred to herein as an “immunogenic NH polypeptide” and a pharmaceutically acceptable carrier.

The immunogenic NH polypeptides, as used herein, comprise an epitope-bearing portion of an NH protein. An immunogenic NH polypeptide is a polypeptide that is capable of producing antibodies with a specific binding affinity to N. helminthoeca in a subject to whom the immunogenic composition has been administered.

Also disclosed is a vaccine comprising an immunogenic NH polypeptide, together with a pharmaceutically acceptable diluent, carrier, or excipient, wherein the immunogenic NH polypeptide is present in an amount effective to elicit a beneficial immune response in a subject to NH. The immunogenic NH polypeptide may be obtained as described above and using methods well known in the art.

In another embodiment, the present invention relates to a vaccine comprising an NH nucleic acid (e.g., DNA) or a segment thereof (e.g., a segment encoding an immunogenic NH polypeptide) together with a pharmaceutically acceptable diluent, carrier, or excipient, wherein the nucleic acid is present in an amount effective to elicit, in a subject, a beneficial immune response to NH. The NH nucleic acid may be obtained as described above and using methods well known in the art.

In a further embodiment, the present invention relates to a method of producing an immune response which recognizes NH in a host, comprising administering to the host one or more of the above-described immunogenic NH polypeptides.

In some embodiments, the host or subject to be protected is a member of the Canidae family including domestic dogs, foxes, and coyotes.

Also disclosed is a method of preventing or inhibiting salmon poisoning disease (SPD) in a subject comprising administering to the subject the above-described vaccine, wherein the vaccine is administered in an amount effective to prevent or inhibit SPD. The vaccine of the invention is used in an amount effective depending on the route of administration. Although intra-nasal, subcutaneous or intramuscular routes of administration are suitable, the vaccine of the present invention can also be administered by an oral, intraperitoneal or intravenous route. One skilled in the art will appreciate that the amounts to be administered for any particular treatment protocol can be readily determined without undue experimentation. Suitable amounts are within the range of 2 μg of the NH vaccine per kg body weight to 100 micrograms per kg body weight (preferably, 2 μg to 50 μg, 2 μg to 25 μg, 5 μg to 50 μg, or 5 μg to 10 μg).

Examples of vaccine formulations including antigen amounts, route of administration and addition of adjuvants can be found in Kensil, Therapeutic Drug Carrier Systems 13:1-55 (1996), Livingston et al., Vaccine 12:1275 (1994), and Powell et al., AIDS RES, Human Retroviruses 10:5105 (1994). The disclosed vaccine may be employed in such forms as capsules, liquid solutions, suspensions or elixirs for oral administration, or sterile liquid forms such as solutions or suspensions. Any inert carrier may be used, such as saline, phosphate-buffered saline, or any such carrier in which the vaccine has suitable solubility properties. The vaccines may be in the form of single dose preparations or in multi-dose flasks which can be used for mass vaccination programs. Reference is made to Remington's Pharmaceutical Sciences, Mack Publishing Co., Easton, Pa., Osol (ed.) (1980); and New Trends and Developments in Vaccines, Voller et al (eds), University Park Press, Baltimore, Md. (1978), for methods of preparing and using vaccines.

The disclosed vaccines may further comprise adjuvants which enhance production of antibodies and immune cells. Such adjuvants include, but are not limited to, various oil formulations such as Freund's complete adjuvant (CFA), the dipeptide known as MDP, saponins (ex, QS-21, U.S. Pat. No. 5,047,540), aluminum hydroxide, or lymphatic cytokines. Freund's adjuvant is an emulsion of mineral oil and water which is mixed with the immunogenic substance. Although Freund's adjuvant is powerful, it is usually not administered to humans. Instead, the adjuvant alum (aluminum hydroxide) may be used for administration to a human. Vaccine may be absorbed onto the aluminum hydroxide from which it is slowly released after injection. The vaccine may also be encapsulated within liposomes according to Fullerton, U.S. Pat. No. 4,235,877.

In some embodiments, disclosed herein is a method of detecting an infection with N. helminthoeca in a Canidae patient comprising the steps of:

(a) providing a serum sample from the patient;

(b) providing an isolated or purified N. helminthoeca protein selected from the group consisting of P51, NSP1, NSP2, NSP3, and SSA;

(c) contacting the serum sample with the isolated or purified N. helminthoeca protein; and

(d) assaying for the formation of a complex between antibodies in the serum sample and the isolated or purified N. helminthoeca protein, wherein formation of said complex is indicative of infection with N. helminthoeca.

In some embodiments, disclosed herein is a method of detecting an infection with N. helminthoeca in a Canidae patient comprising the steps of:

(a) providing a serum sample from the patient;

(b) providing one or more antibodies that specifically bind to a N. helminthoeca polypeptide, wherein the N. helminthoeca polypeptide is selected from the group consisting of P51, NSP1, NSP2, NSP3, and SSA;

(c) contacting the serum sample with the one or more antibodies; and

(d) assaying for the formation of a complex between N. helminthoeca proteins in the serum sample and the one or more antibodies, wherein formation of said complex is indicative of infection with N. helminthoeca.

In some embodiments, disclosed herein is a method of detecting N. helminthoeca polypeptides in a test sample comprising
(a) contacting one or more antibodies that specifically bind to a N. helminthoeca polypeptide with the test sample under conditions that allow polypeptide/antibody complexes to form; wherein the N. helminthoeca polypeptide comprises the amino acid sequence of one or more of the following: SEQ ID NO: 1, SEQ ID NO: 2, SEQ ID NO: 3, SEQ ID NO: 4, or SEQ ID NO: 5;
(b) detecting polypeptide/antibody complexes; wherein the detection of polypeptide/antibody complexes is an indication that an N. helminthoeca polypeptide is present in the test sample.

In some embodiments, the one or more antibodies are monoclonal antibodies, polyclonal antibodies, Fab fragments, Fab′ fragments, Fab′-SH fragments, F(ab′)₂ fragments, Fv fragments, or single chain antibodies.

In some embodiments, disclosed herein is a method of detecting antibodies specific for N. helminthoeca comprising:
(a) contacting a test sample with one or more isolated. N. helminthoeca polypeptides under conditions that allow polypeptide/antibody complexes to form; wherein the N. helminthoeca polypeptide comprises the amino acid sequence of one or more of the following: SEQ ID NO: 1, SEQ ID NO: 2, SEQ ID NO: 3, SEQ ID NO: 4, or SEQ ID NO: 5;
(b) assaying for the formation of a complex between antibodies in the test sample and the one or more N. helminthoeca polypeptides; wherein the formation of said complex is an indication that antibodies specific for N. helminthoeca are present in the test sample.

In some embodiments, the one or more isolated N. helminthoeca polypeptides is at least 85% identical to SEQ ID NO: 1, SEQ ID NO: 2, SEQ ID NO: 3, SEQ ID NO: 4, SEQ ID NO: 5 (or a functional derivative thereof).

In some embodiments, the one or more isolated N. helminthoeca polypeptides comprises an immunoreactive fragment that has a length of from 6 amino acids to less than the full length of the N. helminthoeca protein and comprises 6 or more consecutive amino acids of an amino acid sequence that is set forth in SEQ ID NO: 1, SEQ ID NO: 2, SEQ ID NO: 3, SEQ ID NO: 4, and/or SEQ ID NO: 5.

In some embodiments, disclosed herein is an isolated or purified outer membrane protein of N. helminthoeca, a variant of said outer membrane protein, or an immunogenic fragment of said outer membrane protein, wherein said outer membrane protein is P51, NSP1, NSP2, NSP3, SSA, or a fragment thereof.

In some embodiments, disclosed herein is an expression vector for transformation of a host cell, said vector comprising an isolated polynucleotide that encodes an outer membrane protein of N. helminthoeca, a variant of said outer membrane protein, or an immunogenic fragment of said outer membrane protein, wherein said outer membrane protein is P51, NSP1, NSP2, NSP3, SSA, or a fragment thereof. In some embodiments, disclosed herein is a host cell comprising the expression vector comprising an isolated polynucleotide that encodes an outer membrane protein of N. helminthoeca, a variant of said outer membrane protein, or an immunogenic fragment of said outer membrane protein, wherein said outer membrane protein is P51, NSP1, NSP2 NSP3, SSA, or a fragment thereof.

In some embodiments, disclosed herein is an isolated outer membrane protein of N. helminthoeca consisting of a sequence that is at least 85% identical to SEQ ID NO: 1, SEQ ID NO: 2, SEQ ID NO: 3, SEQ ID NO: 4, or SEQ ID NO: 5. In some embodiments, to disclosed herein is an isolated outer membrane protein of N. helminthoeca consisting of a sequence that is at least 90% identical to SEQ ID NO: 1, SEQ ID NO: 2, SEQ ID NO: 3, SEQ ID NO: 4, or SEQ ID NO: 5. In some embodiments, disclosed herein is an isolated outer membrane protein of N. helminthoeca consisting of a sequence that is at least 95% identical to SEQ ID NO: 1, SEQ ID NO: 2, SEQ ID NO: 3, SEQ ID NO: 4, or SEQ NO: 5.

In some embodiments, disclosed herein is an isolated outer membrane protein of claim 1, wherein the polypeptide contains an immunoreactive fragment that is 6 or more consecutive amino acids from the following sequences: (1) SEQ ID NO: 1; (2) SEQ ID NO: 2; (3) SEQ ID NO: 3; (4) SEQ ID NO: 4; (5) SEQ ID NO: 5; or any combination of the sequences (1)-(5).

In some embodiments, disclosed herein is a kit for detecting N. helminthoeca in a subject, said kit comprising an N. helminthoeca protein, an antigenic fragment of an N. helminthoeca protein, or both; wherein the N. helminthoeca protein is selected from the group consisting of P51, NSP1, NSP2, NSP3, and SSA. In some embodiments the kit further comprises a biomolecule for detecting interaction between the N. helminthoeca protein reagent and antibodies in a bodily sample of the animal.

In some embodiments, disclosed herein is a kit for detecting N. helminthoeca in a subject, said kit comprising an N. helminthoeca protein, an antigenic fragment of an N. helminthoeca protein, or both; wherein the N. helminthoeca protein is selected from the group consisting of P51, NSP1, NSP2, NSP3, and SSA.

In some embodiments, disclosed herein is a reagent kit for detecting infection with N. helminthoeca in a subject comprising one or more antibodies that specifically bind to a N. helminthoeca polypeptide, wherein the N. helminthoeca polypeptide is selected from the group consisting of P51, NSP1, NSP2, NSP3, and SSA.

A number of embodiments of the invention have been described. Nevertheless, it will be understood that various modifications may be made without departing from the spirit and scope of the invention. Accordingly, other embodiments are within the scope of the following claims.

### EXAMPLES

The following examples are set forth below to illustrate the compounds, compositions, methods and results according to the disclosed subject matter. These examples are not intended to be inclusive of all aspects of the subject matter disclosed herein, but rather to illustrate representative compounds, compositions, methods and results. These examples are not intended to exclude equivalents and variations of the present invention which are apparent to one skilled in the art.

### Example 1

### Analysis of Complete Genome Sequence and Major Surface Antigens of Neorickettsia helminthoeca, Causative Agent of Salmon Poisoning Disease

Neoricketts helminthoeca, a type species of the genus Neorickettsia, is an endosymbiont of digenetic trematodes of veterinary importance. Upon ingestion of salmonid fish parasitized with infected trematodes, canids develop salmon poisoning disease (SPD), an acute febrile illness that is particularly severe and often fatal in dogs without adequate treatment. The complete genome sequence of N. helminthoeca was determined and analyzed: a single small circular chromosome of 884,232 bp encoding 774 potential proteins. N. helminthoeca is unable to synthesize lipopolysaccharides and most amino acids, but is capable of synthesizing vitamins, cofactors, nucleotides, and bacterioferdtin. N. helminthoeca is, however, distinct from majority of the family Anaplasmataceae to which it belongs, as it encodes nearly all enzymes required for peptidoglycan biosynthesis, suggesting its structural hardiness and inflammatory potential. Using sera from dogs that were experimentally infected by feeding with parasitized fish or naturally infected in Southern California, western blotting analysis revealed that among five predicted N. helminthoeca outer membrane proteins, P51 and strain-variable surface antigen were uniformly recognized. These results aid in understanding pathogenesis, prevalence of N. helminthoeca infection among trematodes, canids, and potentially other animals in nature to develop effective SPD diagnostic and preventive measures. Recent progresses in large-scale genome sequencing have been uncovering broad distribution of Neorickettsia spp., the comparative genomics will facilitate understanding of biology and the natural history of these elusive environmental bacteria.

N. helminthoeca can stably be continuously cultured in a DH82 canine macrophage cell line for up to 3 months with inoculation of infected DH82 cells inducing a more severe form of the disease in dogs. This advancement has allowed for the investigation of genetic and antigenic properties of N. helminthoeca and clarification of its relationship to other members of the family Anaplasmataceae leading to reclassification of N. helminthoeca, N. risticii, N. sennetsu, and SF agent into their own clade (Table 1). In this study, experiments were conducted to synthesize the whole N. helminthoeca bacterial genome, determine, clone, and purify antigenic outer membrane proteins (OMPs), probe these recombinant OMPs using experimentally and clinically SPD infected dog sera, and determine specific highly antigenic, surface exposed regions of these outer membrane proteins that are phylogenetically divergent from species closely related to N. helminthoeca, namely N. risticii and N. sennetsu.

In this example, three results were sought: (1) determine the complete genome of N. helminthoeca and compare with closely-related N. risticii and N. sennetsu genomes; (2) determine, clone, and purify putative immunodominant major outer membrane proteins (OMPs); and (3) test immunoreactivity of these recombinant OMPs using sera from dogs that were experimentally or naturally infected with N. helminthoeca.

### Results and Discussion

General Features of the Genome

The genome of N. helminthoeca Oregon consists of a single double-stranded circular chromosome spanning 884,232 bp, which is similar to those of N. risticii (Lin et al., 2009) and N. sennetsu (Dunning Hotopp et al., 2006) (Table 2), and smaller than those of other members in the family Anaplasmataceae (approximately 1.0-1.5 Mbp) (Dunning Hotopp et al., 2006). G+C content of N. helminthoeca genome is 41.7% (Table 2), which is similar to those of other Neorickettsia and Anaplasma spp., but greater than those (approximately 30%) of Ehrlichia spp. and Wolbachia spp. (Dunning Hotopp et al., 2006). The replication origin of N. helminthoeca (FIG. 2) was predicted based on one of the GC-skew shift points, and the region between hemE (uroporphyrinogen decarboxylase, NHE_RS00005) and an uncharacterized phage protein (NHE_RS04160) as described in N. risticii (Lin et al., 2009), N. sennetsu (Dunning Hotopp et al., 2006) and other members in the family Anaplasmataceae (Ioannidis et al., 2007).

The N. helminthoeca genome encodes one copy each of the 5S, 16S, and 23S rRNA genes, which are separated in 2 loci with the 5S and 23S rRNA genes forming an operon (FIG. 2, red bars in 3^{rd} circle from outside) as in other sequenced members in the family Anaplasmataceae (Massung, et al., 2002; Dunning Hotopp et al., 2006). Thirty-three tRNA genes are identified, which include cognates for all 20 amino acids (Table 2). The numbers of tRNA genes are identical to other Neorickettsia spp., and similar to other members in the family Anaplasmataceae (Dunning Hotopp et al., 2006; Lin et al., 2009), or other bacteria with a single rrn operon (Lee et al., 2009).

With 827 protein- and RNA-coding genes (FIG. 2, Table 2), N. helminthoeca has a smaller number of predicted genes as compared to other members in the family Anaplasmataceae, including Ehrlichia, Anaplasma, and Wolbachia endosymbionts of insects or nematodes, each of which have around 1,000 or more genes (Crossman, 2006; Dunning Hotopp et al., 2006; Lin et al., 2009). Among the 774 predicted protein-coding open reading frames (ORB), 548 genes are assigned with probable functions based on sequence similarity searches. Approximately 29% of the predicted ORFs (226 genes) in the genome are annotated as hypothetical proteins, either with conserved domains or of unknown functions (Table 3).

Comparison of Genomic Contents Among Neorickettsia Species

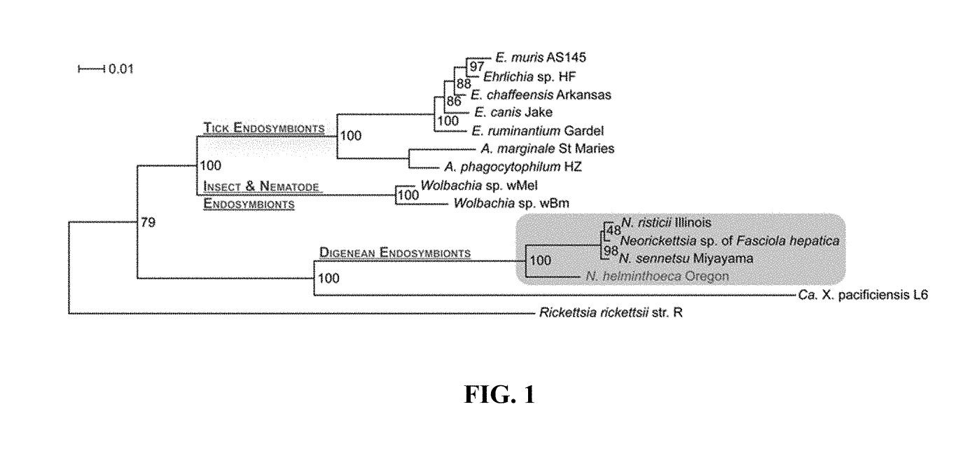

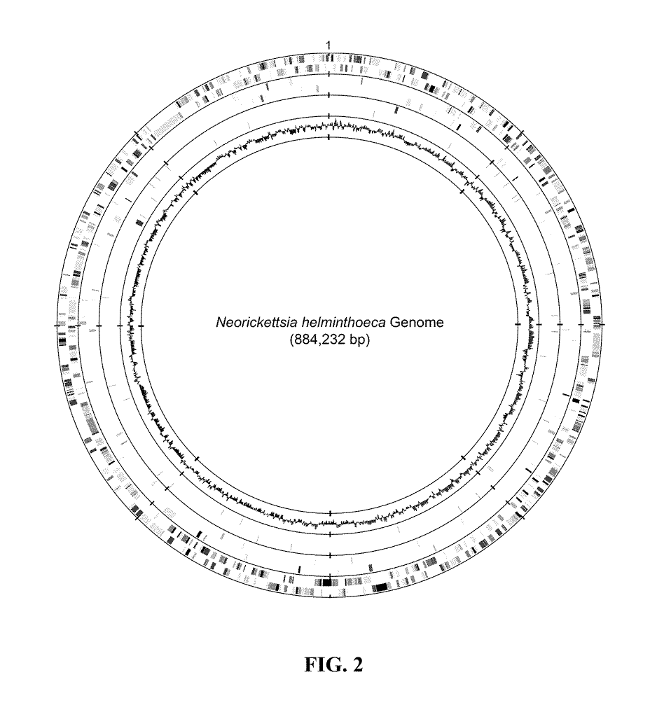

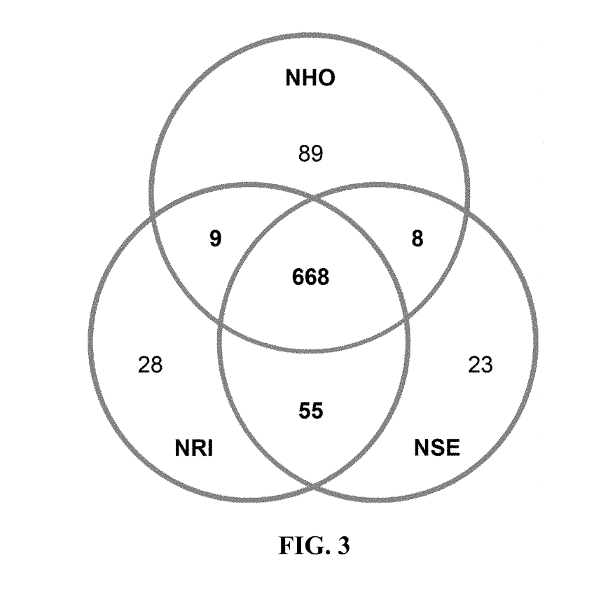

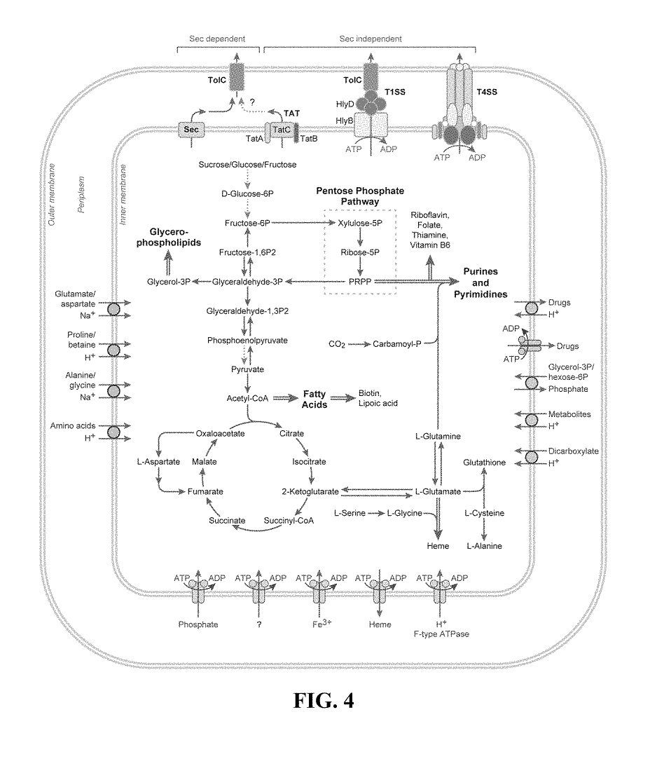

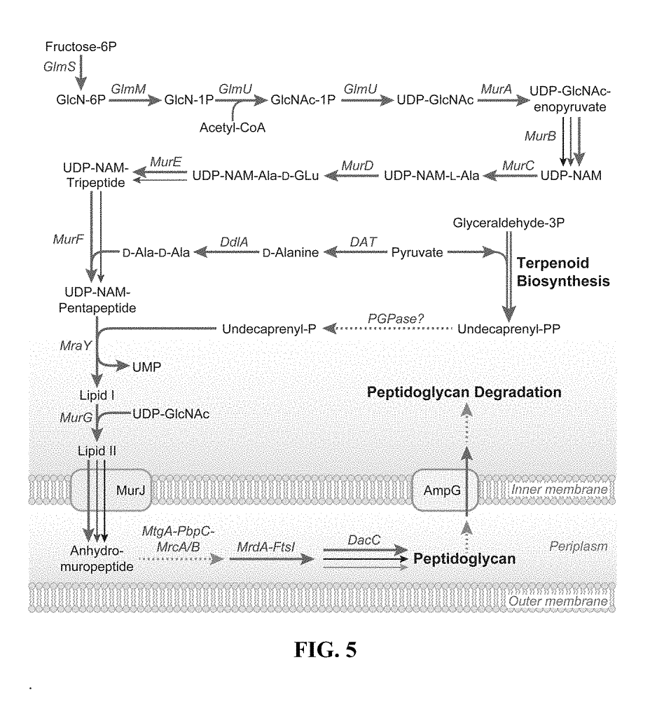

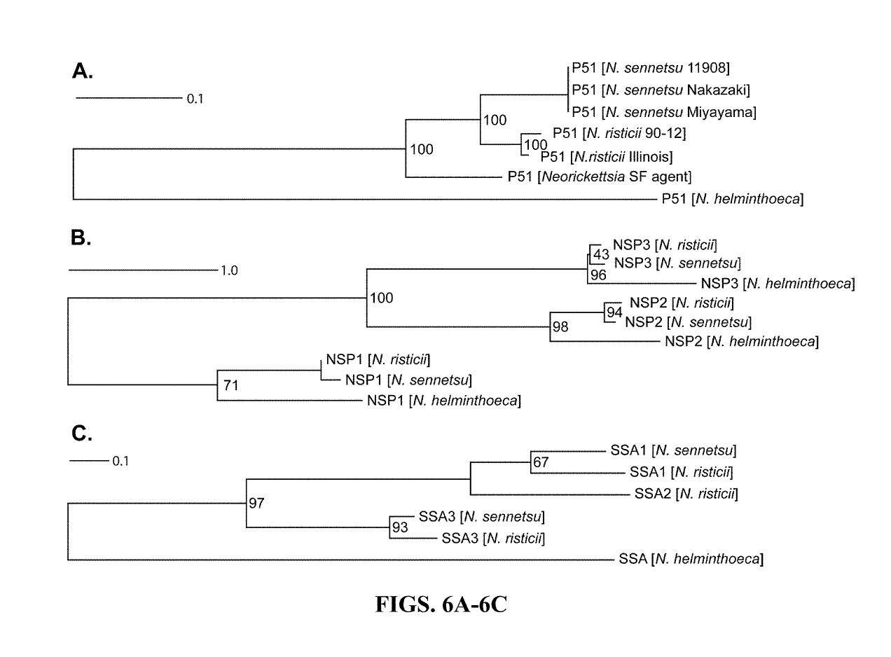

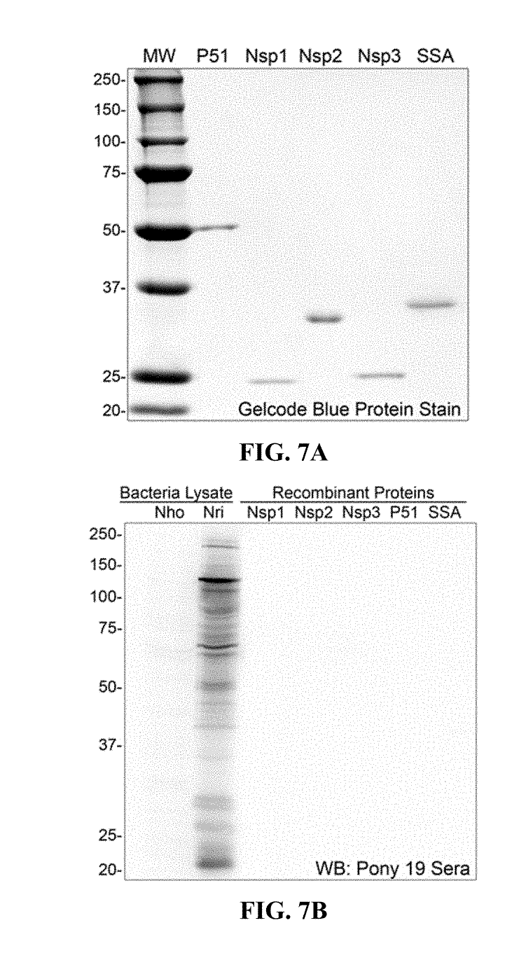

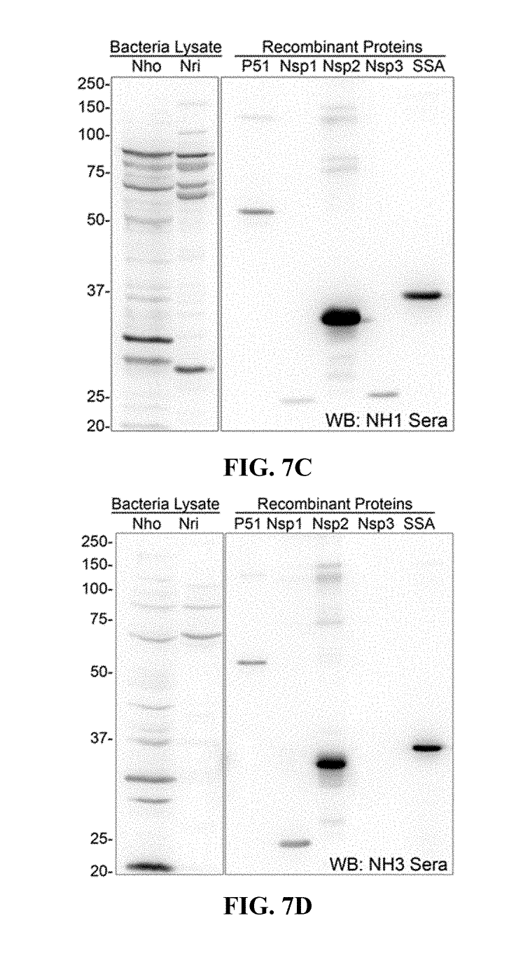

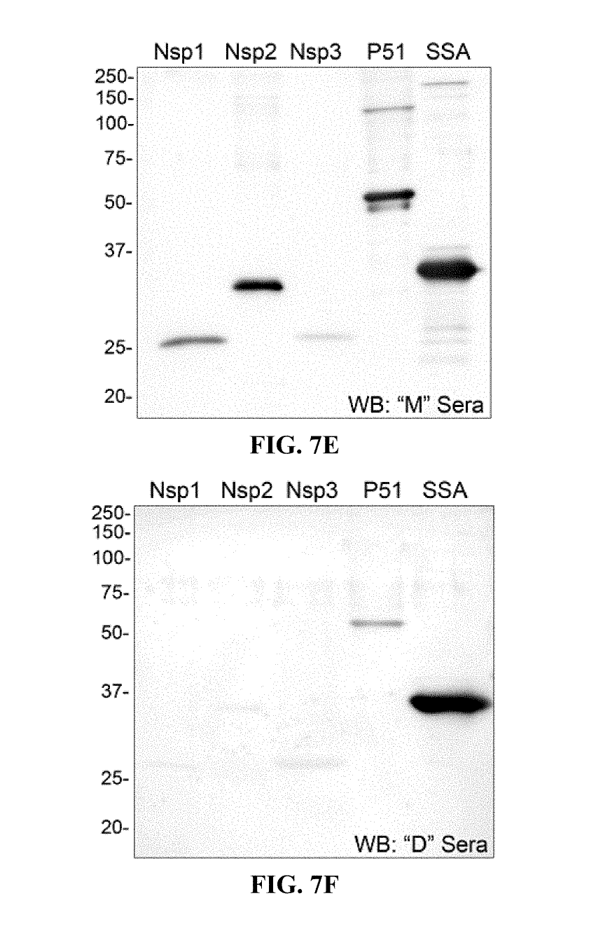

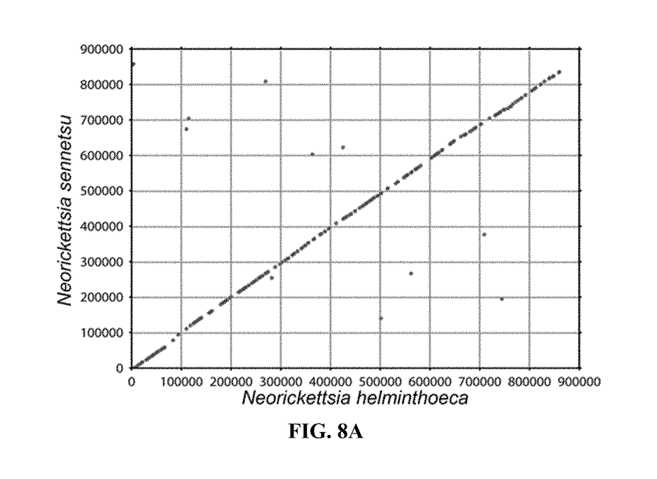

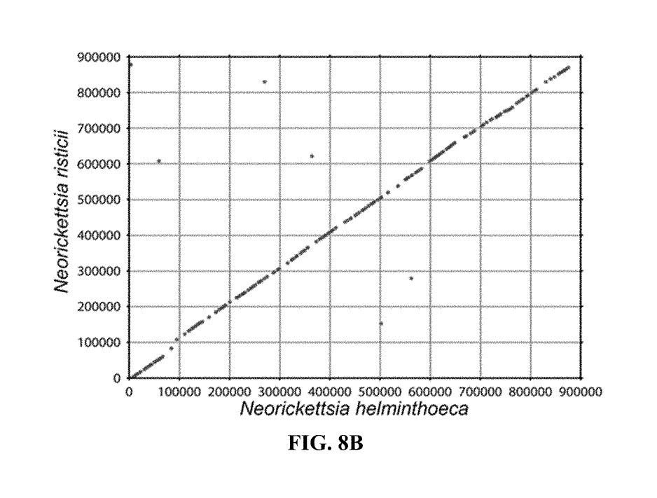

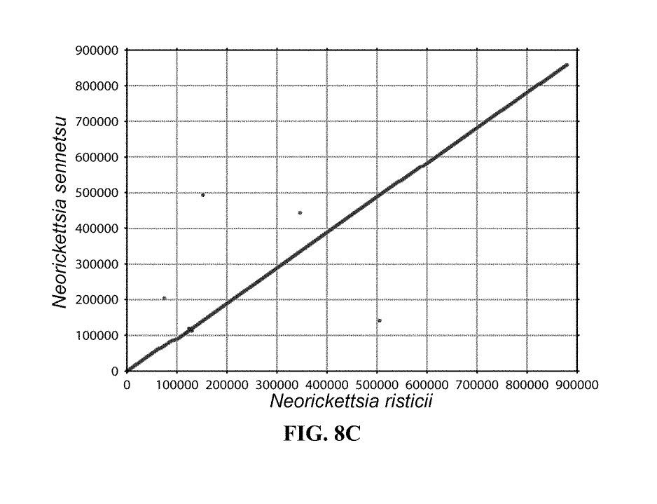

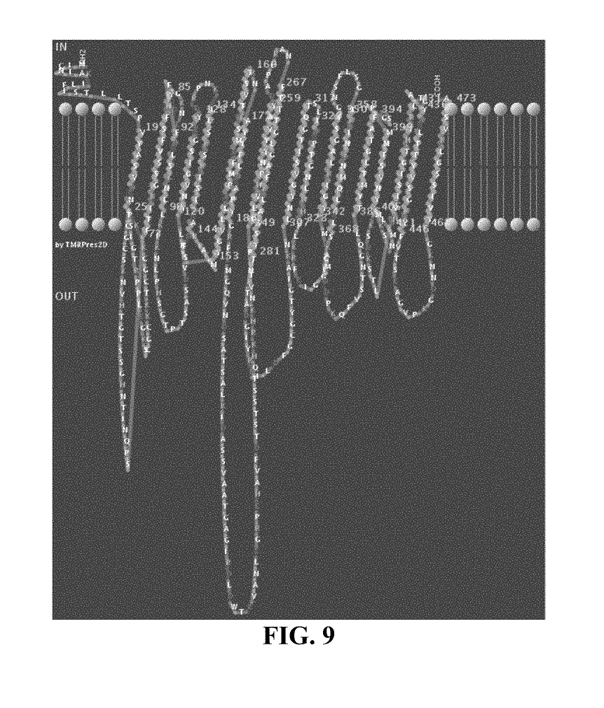

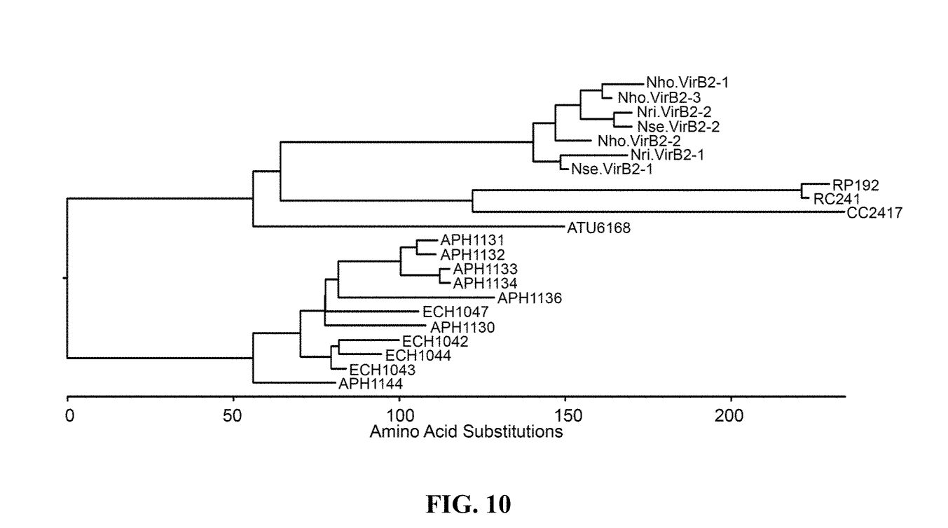

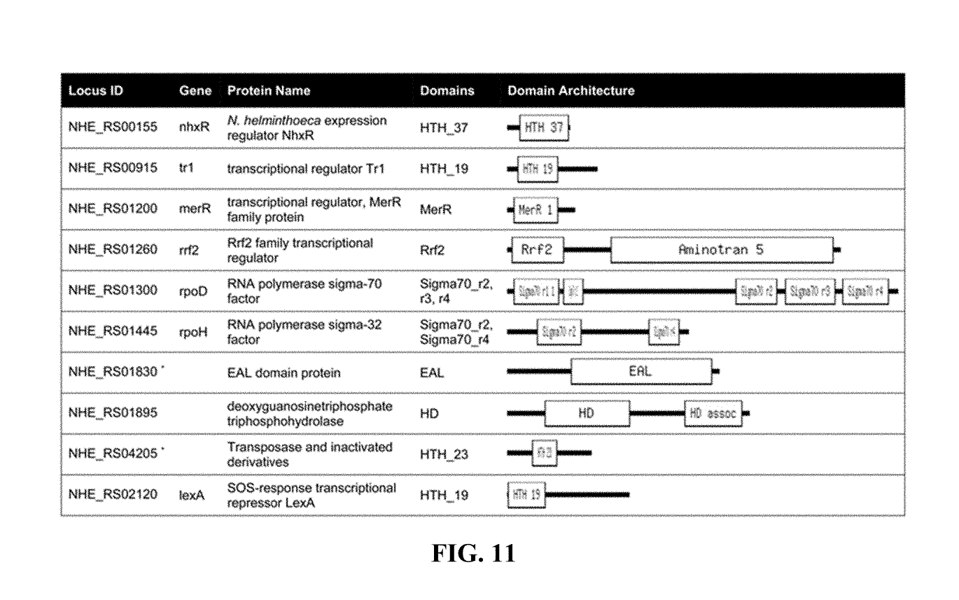

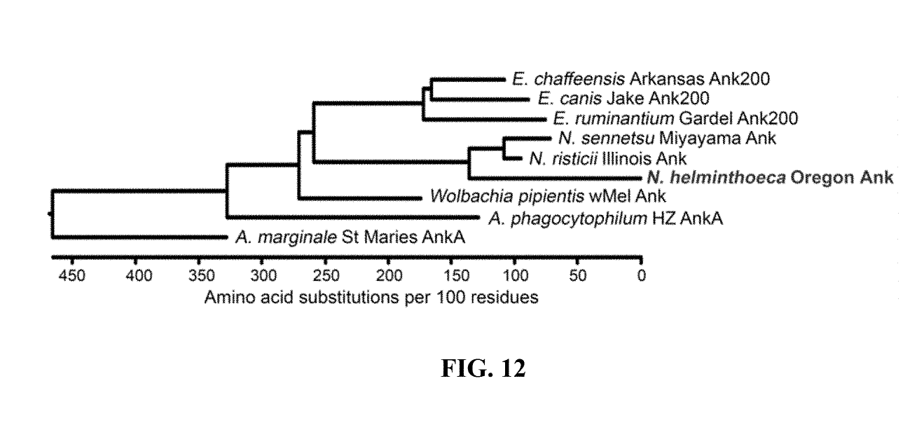

Previous studies have shown that Anaplasma spp. and Ehrlichia spp. have a single large-scale symmetrical inversion (X-alignment) near the replication origin, which is possibly mediated by duplicated rho genes (Dunning Hotopp et al., 2006; Frutos et al., 2007; Nene and Kole, 2009). In addition, Anaplasma and Wolbachia spp. have extensive genomic rearrangement throughout the genome (Wu et al., 2004; Dunning Hotopp et al., 2006). However, the synteny is highly conserved and such genomic rearrangements or a large scale inversion are not detected among N. helminthoeca, N. sennetsu, and N. risticii (FIG. 8), and rho is not duplicated in three sequenced Neorickettsia spp. in agreement with the 16S rRNA divergence (FIG. 1), N. helminthoeca exhibits multiple synteny divergence from N. risticii and N. sennetsu (FIG. 8).

In order to compare the genomic contents among Neorickettsia spp., 2- and 3-way comparisons were performed using reciprocal BLASTP algorithm with E-value<1e⁻¹⁰, and homologous protein clusters were constructed. Three-way comparison among Neorickettsia spp. showed that >86% (668 of total 774 protein-coding ORFs) of N. helminthoeca proteins are conserved with N. risticii and N. sennetsu (Table 3 and Table 5). The vast majority (>82%, 548/668 ORFs) of these conserved proteins are associated with housekeeping functions and likely essential for Neorickettsia survival (Table 3). Two-way comparisons revealed that N. risticii and N. sennetsu share an additional 55 conserved proteins, whereas N. helminthoeca shares very limited numbers of orthologs (<10 proteins) with N. risticii or N. sennetsu (FIG. 3). The result of the 2-way and 3-way comparisons is consistent with the relationship of the species revealed through 16S rRNA-based phylogeny and whole-genome synteny analysis.

The three Neorickettsia spp. are transmitted by distinct trematodes and cause severe diseases at high mortality in different mammalian hosts (Table 1) (Cordes et al., 1986; Dutta et al., 1988; Rikihisa et al., 1991; Rikihisa et al., 2004; Rikihisa et al., 2005; Gibson and Rikihisa, 2008; Lin et al., 2009). We, therefore, analyzed the species-specific genes based on the 2- and 3-way comparisons. There are 89 species-specific proteins in N. helminthoeca as compared to 23 and 28 in N. risticii and N. sennetsu, respectively (Tables 6-8). Of the genes unique to N. helminthoeca, more than half of them (50/89 ORFS) are hypothetical proteins without assigned functions (Table 6). Among the N. helminthoeca-specific proteins with assigned functions, ˜38% (15/39 ORFs) are involved in peptidoglycan biosynthesis that are absent in N. risticii and N. sennetsu (Table 6 and FIG. 5), and six proteins are categorized as transporters for iron and other substrates (Table 6). The genomic loci encoding these unique ORFs are distributed throughout N. helminthoeca genome and not clustered in certain islands (FIG. 2, 2^{nd} circle from outside). Blast searches using these N. helminthoeca-specific proteins against NCBI protein database excluding Neorickettsia spp. showed that only 29 of them match to proteins in other genera, and the majority of them (19, 65.5%) belong to α-proteobacteria (Table 6). However, whether these proteins are the results of horizontal gene transfer or mutations/deletions from the ancestors of Neorickettsia spp. remains to be determined.

Metabolism

Except for peptidoglycan biosynthesis, most metabolic pathways, transcription, translation, and regulatory functions, are highly conserved in N. helminthoeca compared to N. sennetsu and N. risticii (summarized in FIG. 4, Tables 3 and 5) (Dunning Hotopp et al., 2006; Lin et al., 2009).

Central metabolic pathways. Analysis of the metabolic pathways based on Kyoto Encyclopedia of Genes and Genomes (KEGG, http://www.kegg.jp) and BioCyc (http://biocyc.org/) indicates that, similar to other members in the family Anaplasmataceae, N. helminthoeca encodes pathways for aerobic respiration, including the tricarboxylic acid (TCA) cycle and the electron transport chain, but it is unable to use glucose, fructose, or fatty acids directly as a carbon or energy source, since essential enzymes for the utilization of these substrates such as hexokinases, the first enzyme in the glycolysis pathway that converts glucose to glucose-6-phosphate, and pyruvate kinase that converts phosphoenolpyruvate to pyruvate, are not identified (FIG. 4). It is likely that N. helminthoeca can synthesize ATP from glutamine as N. risticii, N. sennetsu, or E. chaffeensis does (Weiss et al., 1989; Cheng et al., 2014), since it encodes carbamoyl phosphate synthase (carA/B, NHE_RS00875/NHE_RS02090) and bifunctional glutamate synthase □ subunit/2-polyprenylphenol hydroxylase (GS/PH, NHE_RS02780). These enzymes can convert glutamine to ammonia and glutamate (FIG. 4), and glutamate can be further converted by glutamate dehydrogenase (NHE_RS02165) to 2-ketoglutarate, which enters the TCA cycle for energy production.

Amino acids, nucleotides, fatty acids, and cofactor biosynthesis. Like other Neorickettsia, Ehrlichia, and Anaplasma spp. (Dunning Hotopp et al., 2006; Lin et al., 2009), N. helminthoeca synthesizes very limited amino acids including alanine, aspartate, glycine, glutamate, and glutamine (FIG. 4 and Table 9). Since they are converted from other amino acids or metabolic intermediates, N. helminthoeca must transport most amino acids from its host as discussed further below (Table 11). However, as other members of the family Anaplasmataceae, analysis of KEGG pathways showed that most enzymes are identified for the biosynthesis of fatty acids and certain phospholipids, including phosphatidylglycerol, phosphatidylserine, phosphatidylethanolamine, and myo-inositol-phosphates (FIG. 4).

Similar to all other sequenced members of Anaplasmataceae (Dunning Hotopp et al., 2006), N. helminthoeca encodes a nonoxidative pentose-phosphate pathway that utilizes glyceraldehyde-3-phosphate to produce pentose for nucleotide and cofactor biosynthesis. Accordingly, N. helminthoeca encodes complete pathways for de novo purine and pyrimidine biosynthesis, and is capable of synthesizing most vitamins or cofactors, such as biotin, folate, FAD, NAD, and protoheme (FIG. 4 and Table 10). Overall, N. helminthoeca encodes large number of genes involved in the biosynthesis of cofactors, vitamins and nucleotides (17.2%, 133 of total 774 protein-coding ORFs), similar to other members of Anaplasmataceae like Ehrlichia (13.4%, 149/1115 ORFs in E. chaffeensis), Anaplasma (10.6%, 145/1370 ORFs in A. phagocytophilum) (Dunning Hotopp et al., 2006), and Wolbachia endosymbionts of the insects or nematodes (9.4%, 120/1271 in Wolbachia pipientis wMel) (Foster et al., 2005; Brownlie et al., 2009). Unlike tick-borne members in the family Anaplasmataceae (Ehrlichia and Anaplasma spp.), Neorickettsia spp. are maintained throughout the life cycle of the trematodes (Greiman et al., 2016) (FIG. 1). The presence of these biosynthesis pathways suggests that N. helminthoeca do not need to compete with the host for the essential vitamins and nucleotides, which is likely beneficial for their survival especially in invertebrate hosts.

Transporters and porins. To compensate for the incomplete biosynthesis or metabolic pathways, the N. helminthoeca genome encodes several orthologs involved in cytoplasmic membrane transport systems that can supply the necessary amino acids, metabolites, and ions, as analyzed by TransAAP (Transporter Automatic Annotation Pipeline, http://www.membranetransport.org/) (FIG. 4 and Table 11) (Ren et al., 2007; Ren and Paulsen, 2007). Transporters for acetyl-CoA involved in many metabolic pathways and glycerol-3-phosphate in phospholipid biosynthesis are identified in N. helminthoeca genome (Table 11). Transport systems for phosphates (pstA/B/C/S), cations, anions, organic ions, and multidrug resistance pumps are also present (Table 11). Putative amino acid transporters for alanine, glycine, proline, and dicarboxylate amino acids (glutamate or aspartate family) can be found (Table 11). However, since very few amino acids can be synthesized in N. helminthoeca, more transporters are required; it is possible that some ATP-binding cassette (ABC)-type transporters with no assigned functions or porins discussed below could act as transporters for amino acids as well as metabolites for protein synthesis and energy production. Orthologs of most identified transporters are conserved in N. risticii and N. sennetsu genomes (Table 5 and 11), except for few N. helminthoeca-specific transporters listed in Table 6. Unlike Rickettsia spp. (Winkler, 1976), but similar to all other sequenced members of the Family Anaplasmataceae, N. helminthoeca does not encode translocases for ATP (ATP:ADP antiporters) or NADH, so it likely relies on its own ATP production or encodes unique ATP acquisition mechanisms.

Gram-negative bacteria also express porins spanning their outer membranes that enable the transport of hydrophilic and large molecules, such as amino acids, sugars, and other nutrients (Nikaido, 2003). Similar to other members of the Anaplasmataceae that have limited capabilities of amino acids biosynthesis, intermediary metabolism, and glycolysis, nutrient uptake in these bacteria necessitates pores or channels in the bacterial outer membrane (Huang et al., 2007; Kumagai et al., 2008; Gibson et al., 2010). Previous studies have determined that the major outer membrane proteins, including A. phagocytophilum P44s (Huang et al., 2007), E. chaffeensis P28/OMP-1F (Kumagai et al., 2008), and N. sennetsu P51 (Gibson et al., 2010), possess porin activities as determined by a proteoliposome swelling assay, which allow the diffusion of L-glutamine, the monosaccharides arabinose and glucose, the disaccharide sucrose, and even the tetrasaccharide stachyose. N. helminthoeca encodes a P51 protein (NHE_RS00965) that shares 60% amino acid sequence similarity with N. sennetsu P51 protein (FIG. 6A). Prediction of the two-dimensional structure of N. helminthoeca P51 using PRED-TMBB (http://biophysics.biol.uoa.gr/PRED-TMBB/) (Bagos et al., 2004) showed that P51 protein contains 18 transmembrane domains with a discrimination value of 2.949 (FIG. 9), to suggesting that it is a β-barrel protein localized to the outer membrane similar to N. sennetsu P51 (Gibson et al., 2010). Therefore, it is likely that N. helminthoeca P51 can function as a porin for nutrient uptake from the host.

DNA, RNA, protein synthesis, and DNA repair, N. helminthoeca encodes proteins necessary for DNA replication, RNA synthesis and degradation, and ribosomal proteins. Although N. helminthoeca encodes proteins required for homologous recombination, including RecA/RecF (but not RecBCD) pathways (Lin et al., 2006) and RuvABC complexes for Holliday junction recombination as other members of the family Anaplasmataceae (Table 12), it has the least amount of enzymes involved in DNA repair compared to other members of the family Anaplasmataceae including N. sennetsu and N. risticii (7 in N. helminthoeca vs. 9 in N. sennetsu, 12 in E. chaffeensis, and 13 in A. phagocytophilum, Table 12) (Dunning Hotopp et al, 2006; Lin et al., 2009). N. helminthoeca lacks most genes required for mismatch repair, nucleotide excision repair (NER, such as uvrABC for UV-induced DNA damage), various glycosylases for base excision repair (BER), and DNA photolyases, which is an alternative mechanism to repair UV-damaged DNA identified in E. chaffeensis, A. phagocytophilum, and N. risticii (Dunning Hotopp et al., 2006; Lin et al., 2009).

Pathogenesis

Although SPD was recognized more than two centuries ago, the causative agent N. helminthoeca was only stably cultured in canine cell line in 1990 (Rikihisa et al., 1991), and there are little information available regarding the molecular determinants of N. helminthoeca to invade and cause severe disease in canine hosts. Here, genes and pathways were analyzed that are potentially involved in N. helminthoeca pathogenesis, including protein secretion systems, two-component/one-component regulatory systems, N. helminthoeca-specific genes, and putative membrane proteins or lipoproteins.

Protein secretion systems. Two major pathways exist to secrete proteins across the cytoplasmic membrane in bacteria. The general Secretion route, termed Sec-pathway, catalyzes the transmembrane translocation of proteins in their unfolded conformation, whereupon they fold into their native structure at the trans-side of the membrane (Natale et al., 2008). All major components for the Sec-dependent pathway are identified, including signal recognition particle (SRP) protein, SRP-docking protein FtsY, the cytosolic protein-export chaperone SecB, peripheral associated ATP-dependent motor protein SecA, membrane-embedded protein conducting channel SecYEG, periplasmic protein YajC that involved in preprotein translocase activity, and the membrane complex SecDF that enhances proton motive force (FIG. 4 and summarized under role category “Protein fate” in Table 5). In addition, common chaperones are identified in N. helminthoeca genome, including groEL, groES, dnaK, dnaJ, hscA/B, grpE, and htpG (summarized under role category “Protein fate” in Table 5).

Twin-arginine translocation (Tat)-pathway, which consists of the TatA, TatB, and TatC proteins, can transport folded proteins across the bacterial cytoplasmic membrane by recognizing N-terminal signal peptides harboring a distinctive twin-arginine motif (Lee et al., 2006; Sargent et al., 2006). All genes encoding Tat apparatus are identified in the N. helminthoeca genome (tatA/NHE_RS02000, tatB/NHE_RS02160, and tatC/NHE_RS00490) (FIG. 4 and Table 10) (Gillespie et al., 2015). However, despite the presence of Tat system, no protein substrate containing a putative Tat signal peptide can be identified in N. helminthoeca using both TAT-FIND (http://www.cbs.dtu.dk/services/TatP/) (Bendtsen et al 2005) and PRED-TAT (http://www.compgen.org/tools/PRED-TAT) algorithms (Bagos et al., 2010). Gillespie et al (Gillespie et al., 2015) reported only a single Tat substrate (PetA) in Rickettsia, and suggested that could be due to the substantial differences in signal peptides of Tat substrates in the obligate intracellular bacteria.

Extracellular secretion of various virulence factors across the bacterial cell envelope is one of the major mechanisms by which pathogenic bacteria alter host cell functions, thus enhancing survival of the bacteria and damaging hosts. At least six distinct extracellular protein secretion systems, referred to as type I-VI secretion systems (T1SS-T6SS) (Papanikou et al., 2007; Costa et al., 2015), have been classified in Gram-negative bacteria that secrete effector molecules across two lipid bilayers and the periplasm. Except for T2SS, all double-membrane-spanning secretion systems (T1SS, T3SS, T4SS and T6SS) use a one-step mechanism to transport substrates directly from the bacterial cytoplasm into the extracellular space or into a target cell (Costa et al., 2015). Bioinfomatic analysis shows that, similar to all other sequenced members of the family Anaplasmataceae, N. helminthoeca genome encodes both T1SS and T4SS for secretion of proteins across the membranes, but it lacks homologs of T2SS, T3SS, T5SS, or T6SS components (FIG. 4) (Henderson et al., 2004; Cianciotto, 2005; Bingle et al., 2008). T1SS, a Sec-independent ATP-driven ABC transporter system that bypasses the periplasm, is capable of transporting target proteins carrying a C-terminal uncleaned secretion signal across both inner and outer membranes and into the extracellular medium (Delepelaire, 2004). All of the three components of T1SS, including an inner membrane ATP-binding cassette (ABC) transporter HlyB (NHE_RS00175), a periplasmic membrane fusion protein (MFP) HlyD (NHE_RS04020), and an outer membrane channel protein TolC (NHE_RS03400) are identified in the N. helminthoeca genome (FIG. 4, Table 5, and 10). A previous study reported that several tandem repeat proteins (TRP120, TRP47, and TRP32/VLPT) are T1SS substrates of E. chaffeensis using an E. coli T1SS surrogate system (Wakeel et al., 2011). Current analysis using the T-REKS algorism (Jorda and Kajava, 2009) identified several tandem-repeat containing proteins (not homologous to E. chaffeensis TRPS) like VirB6 and SSAs in all three sequenced Neorickettsia; however, whether these proteins are also secreted by T1SS is unknown (Table 14) (Dunning Hotopp et al., 2006; Lin et al., 2009).

T4SS can translocate bacterial effector molecules into host cells, thus often plays a key role in pathogenesis of Gram-negative host-associated bacteria (Cascales and Christie, 2003; Backert and Meyer, 2006; Gillespie et al., 2010; Christie et al., 2014). In several intracellular bacteria including the family Anaplasmataceae such as E. chaffeensis and A. phagocytophilum, the T4SS is critical for survival and replication inside host cells, by inducing autophagy for nutrient acquisition and inhibition of host cell apoptosis (Niu et al.; 2006; Lin et al., 2007; Niu et al., 2010; Liu et al., 2012; Niu et al., 2012; Lin et al., 2016). In the N. helminthoeca genome, a T4SS encoded by virB/D genes distributed in four separate loci was identified. The organization of virB/D gene clusters is conserved among Neorickettsia spp. as with other Anaplasmataceae, with duplicated genes of virB4, virB8, and virB9, and multiple copies of virB2 and virB6 genes (Tables 5 and 10).

Subcellular fractionation and functional studies have demonstrated that VirB2 is the major pilus component of T4SS extracellular filaments (Cascales and Christie, 2003; Backert and Meyer, 2006). A previous study has confirmed that N. risticii VirB2 was localized at the opposite poles on the bacterial surface (Lin et al., 2009), suggesting that VirB2 might serve as secretion channels for the T4SS apparatus like that of Agrobacterium (Cascales and Christie, 2003), and play critical roles in mediating the interaction with host cells. Analysis of N. helminthoeca genome reveals three copies of virB2 upstream of virB4, whereas N. risticii and N. sennetsu encode two virB2 genes (Table 10) (Lin et al., 2009). Alignment of VirB2 protein sequences indicates that VirB2s of Neorickettsia spp. are closely related to those of other α-proteobacteria like Rickettsia, Agrobacterium, and Caulobacter, but are phylogenetically distinct from VirB2s of E. chaffeensis and A. phagocytophilum that form a separate clade (FIG. 10) (Gillespie et al., 2009; Gillespie et al., 2010). The different numbers of virB2 genes and distinct differences in phylogenetic trees of VirB2 from 16S rRNA gene suggest that virB2 genes might undergo lineage-specific mutations, duplications, or deletions (Gillespie et al., 2010).

Two-component regulatory systems. Two-component regulatory systems (TCRS) are signal transduction systems that allow bacteria to sense and respond rapidly to changing environmental conditions (Mitrophanov and Groisman, 2008; Wuichet et al., 2010). TCRS consists of a sensor histidine protein kinase that responds to specific signals, and a cognate response regulator. Phosphorylation of a response regulator by a cognate histidine kinase changes the biochemical properties of its output domain, which can participate in DNA binding and transcriptional control, perform enzymatic activities, bind RNA, or engage in protein—protein interactions (Gao et al., 2007). TCRS plays a key role in controlling virulence responses in a wide variety of bacterial pathogens (Dorman et al., 2001; Mitrophanov and. Groisman, 2008), including E. chaffeensis and A. phagocytophilum in the family Anaplasmataceae, which encode three pairs of TCRS, including CckA/CtrA, PleC/PleD, and NtrX/NtrY (Cheng et al., 2006; Kumagai et al., 2006; Cheng et al., 2011; Kumagai et al., 2011).

Computational analysis reveals that the three sequenced Neorickettsia spp. encode two pairs of TCRS: CckA/CtrA and PleC/PleD (Table 10). The histidine kinase CckA/response regulator CtrA pair, identified only in α-proteobacteria, also have been demonstrated to coordinate multiple cell cycle events at the transcriptional level in E. chaffeensis to regulate bacterial developmental cycle (Cheng et al., 2011). Different from Ehrlichia and Anaplasma, the three Neorickettsia spp. encode two copies of PleC histidine kinase (NHE_RS00035/NHE_RS02255, Tables 5 and 10) and a one-component signal transduction protein, an EAL domain protein (NHE_RS01830) (FIG. 11 and Table 16) (Ulrich and Zhulin, 2007; Lai et al., 2009; Lin et al., 2009; Romling, 2009; Ulrich and Zhulin, 2010), The response regulator PleD (NHE_RS02155) can function as diguanyl cyclase that produces cyclic diguanylate (c-di-GMP) to regulate cell surface adhesiveness like biofilm or extracellular matrix formation (Tischler and Camilli, 2004), whereas EAL domain protein can function as a diguanylate phosphodiesterase (PDE) that converts c-di-GMP to GMP. They likely function synergistically to regulate surface adhesiveness of Neorickettsia, resulting much smaller morulae sizes and more dispersed bacterial colonies compared to Ehrlichia and Anaplasma (Rikihisa, 1991a). In addition, Neorickettsia spp. do not encodes genes for NtrY/NtrX, which are thought to be involved in nitrogen metabolism and regulation of nitrogen fixation genes like glnA that encodes a glutamine synthase as in E. chaffeensis (Cheng et al., 2014). Despite this, N. helminthoeca encodes GlnA (NHE_RS01490) and ABC dicarboxylate amino acid transporters (NHE_RS00770) that are predicted to take up glutamine (Table 11) similar to E. chaffeensis (Cheng et al., 2014), suggesting regulation of nitrogen metabolism in Neorickettsia spp. is different from Ehrlichia and Anaplasma spp.

One-component regulatory systems and transcriptional regulations. One-component regulatory systems consist of a single protein containing both input and output domains, but lack the phospho-transfer domains of TCRS, and carry out signaling events in prokaryotes (Ulrich et al., 2005; Ulrich and Zhulin, 2007, 2010). This study found that compared to Ehrlichia and Anaplasma, the three Neorickettsia spp. encode more proteins in one-component systems (indicated by asterisks in FIG. 11, based on Microbial Signal Transduction Database at http://mistdb.com) (Ulrich et al., 2005). Other than an EAL domain protein described above and an HD-domain containing deoxyguanosinetriphosphate triphosphohydrolase protein (NHE_RS01895), most one-component regulatory systems of N. helminthoeca as well as N. risticii and N. sennetsu are predicted to be DNA-binding transcriptional regulators (FIG. 11, Table 5).

Perhaps due to the relatively homeostatic intracellular environment of the eukaryotic host cells, members of the order Rickettsiales and Chlamydiaceae have a small number of transcriptional regulators. N. helminthoeca as all other members of the family Anaplasmataceae encodes only two sigma factors: the essential RNA polymerase sigma-70 factor (RpoD, RHE_RS01300) responsible for most RNA synthesis in exponentially growing cells, and sigma-32 factor (RpoH, NHE_RS01445) responsible for expression from heat shock promoters.

N. helminthoeca encodes a putative transcriptional regulator NhxR (N. helminthoeca expression regulator), a 12.5-kDa DNA binding protein (NHE_RS00155) that has 90% amino acid identity with N. risticii NrxR (NRI_RS00145) and N. sennetsu, NsxR (NSE_RS00160). NhxR homologs, A. phagocytophilum ApxR and E. chaffeensis EcxR have shown to regulate the expression of P44 outer membrane proteins and the T4SS, respectively (Wang et al., 2007b; Wang et al., 2007a; Cheng et al., 2008). The other putative transcriptional regulator Tr1 (NHE_RS00915) is homologous to A. phagocytophilum and E. chaffeensis Tr1, which is regulated by ApxR in A. phagocytophilum and located at the upstream of the tandem genes encoding the major outer membrane proteins (OMPs), like Omp-1/Msp-2/P44 expression loci in A. phagocytophilum (Lin et al., 2004) or P28/Omp-1 gene clusters in E. chaffeensis (Ohashi et al., 2001; Wang et al., 2007a; Rikihisa, 2010). However, Tr1 in N. helminthoeca, N. risticii, or N. sennetsu is not located at upstream of any of genes encoding the major OMPs of N. helminthoeca including P51, SSA, or NSPs (Table 4).

The present study identified several other N. helminthoeca DNA-binding regulators, which are conserved in N. risticii and N. sennetsu (FIG. 11 and Table 5) (Lin et al., 2009). These proteins include (1) a putative transcriptional regulator (NHE_RS02120) containing a helix-turn-helix motif and a peptidase S24 LexA-like family domain that are likely involved in the SOS response leading to the repair of single-stranded DNA, (2) a DNA-binding protein with a putative transposase domain (NHE_RS04205), (3) a transcriptional regulator of the MerR (mercuric resistance operon regulator) family (NHE_RS01200), and (4) an Rrf2 family transcriptional regulator with aminotransferase class-V domain (NHE_RS01260) (FIG. 11). Functions of any of them remain to be studied.

Ankyrin domain proteins. Ankyrin-repeat domains (Ank), found predominantly in eukaryotic proteins, are known to mediate protein-protein interactions involved in a multitude of host processes, including cytoskeletal motility, tumor suppression, and transcriptional regulation (Bennett and Baines, 2001; Mosavi et al., 2004). Compared to free-living bacteria, Ank proteins are enriched in facultative and obligate intracellular bacteria of eukaryotes (Jernigan and Bordenstein, 2014). Several studies have shown that the ankyrin repeat-containing protein AnkA of A. phagocytophilum is secreted into host cells by the T4SS and plays an important role in facilitating intracellular infection by activating the Abl-1 protein tyrosine kinase, interacting with the host tyrosine phosphatase SHP-1, or regulation of host cell transcription (Udo et al., 2007; Lin et al., 2007; Garcia-Garcia et al., 2009). In E. chaffeensis, AnkA homolog Ank200 is translocated into the host cell nucleus though a T1SS-dependent manner, and binds to Alu elements and numerous host proteins (Zhu et al., 2009; Wakeel et al., 2011). Four ankyrin-repeat containing proteins were identified in the N. helminthoeca genome (4 in N. risticii and 3 in N. sennetsu) (Table 10). Phylogenetic analysis indicated that N. helminthoeca encodes one Ank protein (NHE_RS00105) that is clustered with E. chaffeensis T1SS substrate Ank200 (11.6% amino acid similarities) (Wakeel et al., 2011) and less related to A. phagocytophilum T4SS substrate AnkA (8.6% amino acid similarities) (Lin et al., 2007) (FIG. 12). However, whether any of these ankyrin repeat-containing proteins of Neorickettsia spp. can be secreted into host cytoplasm by the T1SS or T4SS and regulate host cell functions remain to be determined.

Iron uptake and storage. Iron is an essential element for almost all living organisms, and serves as a cofactor in key metabolic processes including energy generation, electron transport, and DNA synthesis (Skaar, 2010). This study found that the three Neorickettsia spp., E. chaffeensis, and A. phagocytophilum encode proteins for iron transport across inner membranes, including periplasmic Fe³⁺-binding protein FbpA (NHE_RS00045), cytoplasmic membrane permease component FbpB (NHE_RS01265), and cytoplasmic ABC transporter FbpC (PotC, NHE_RS01995) (Table 5). However, homologs to known bacterial siderophore and outer membrane receptors for iron or chelated iron are not identified in these bacteria, suggesting that they might use a unique system to bind and uptake iron from their host. Infection of N. risticii, N. sennetsu, and E. chaffeensis, but not A. phagocytophilum, are inhibited by an intracellular labile iron chelator deferoxamine (Park and Rikihisa, 1992; Barnewall and Rikihisa, 1994; Barnewall et al., 1999), suggesting that these bacteria may utilize different iron-uptake system to obtain iron from the host. Unlike E. chaffeensis and A. phagocytophilum, current analysis found that the three Neorickettsia spp. encode a bacterioferritin (NHE_RS01470) (Table 5, under role category “Transport and binding proteins”), which can capture soluble but potentially toxic Fe²⁺ by compartmentalizing it in the form of a bioavailable ferric mineral inside the protein's hollow cavity. In the family Anaplasmataceae, bacterioferritin is also found in the Wolbachia endosymbiont of insects or nematode (Kremer et al., 2009). This could be due to differences in their life cycle and invertebrate host: the entire life cycles of Neorickettsia and Wolbachia spp. are within trematodes, insects, or nematodes with limited labile iron pools, whereas Ehrlichia and Anaplasma live within mammalian blood cells and tick vectors fed on blood rich in iron (FIG. 1).

Cell Wall Components

Lipopolysaccharide and peptidoglycan. N. helminthoeca lacks all genes encoding lipopolysaccharide (LPS) biosynthesis pathway including lipid A (the core component of LPS) as other sequenced members of the family Anaplasmataceae (Lin and Rikihisa, 2003; Dunning Hotopp et al., 2006; Lin et al., 2009), including the recently sequenced NFh (McNulty et al., 2017). Although few genes involved in LPS biosynthesis were identified in the draft genome of Candidatus “X. pacificiensis”, it was not expected to possess a functional LPS biosynthesis pathway (Kwan and Schmidt, 2013).

Interestingly, nearly all genes involved in peptidoglycan biosynthesis are identified in N. helminthoeca, A. marginale, and Wolbachia wMel (endosymbiont of insect Drosophila melanogaster) or wBm (endosymbiont of nematode Brugia malayi) in the family Anaplasmataceae. On the contrary, only a very limited numbers of genes in peptidoglycan biosynthesis are present in the genomes of N. risticii, N. sennetsu, E. chaffeenis, E. ruminantium, and A. phagocytophilum (FIG. 5). This suggests that the ancestors of the family Anaplasmataceae have undergone independent but parallel loss of the peptidoglycan biosynthetic genes and genome reduction.

Analysis of N. helminthoeca genome suggests that it can perform de novo synthesis of D-Ala-D-Ala from pyruvate, meso-2,6-diaminopimelate (mDAP) from L-Asp, and undecaprenyl-di phosphate (Und-PP) through terpenoid biosynthesis pathways (isopentenyl- and farnesyl-diphosphate). Although undecaprenyl diphosphatase like E. coli phosphatidylglycerophosphatase B (PGPase B, PgpB) homolog was not found in N. helminthoeca, N. helminthoeca encodes two putative PgpA superfamily proteins (NHE_RS00895 and NHE_RS01205) that might function as PGPases to produce Und-P from Und-PP. A flippase (MurJ, NHE_RS02395) that transports anhydromuropeptide into periplasm was also identified in N. helminthoeca (FIG. 5).

The incorporation of anhydromuropeptide subunits into the murein sacculus requires multiple enzymes like MtgA, MrcA/B, FtsI (PbpB), PbpC, MrdA (Pbp2), MrdB, DacF, Pal, MreB/C (Vollmer and Bertsche, 2008; Gillespie et al., 2010); however, only 3 genes encoding MrdA, FtsI (PbpB), and DacC were identified in N. helminthoeca (FIG. 5). In addition, except for an AmpG permease (NHE_RS03475) that can transport components of peptidoglycan into the cytoplasm, N. helminthoeca lacks all necessary enzymes required for the degradation and recycling of peptidoglycan, including lytic transglycosylases (LTs), AmpD, AnmK, LdcA, Mpl, YcjI/G, NagA/B/K/Z, PepD, and MurQ (Gillespie et al., 2010). Furthermore, the T4SS usually encodes specialized LTs that hydrolyze and facilitate the local disruption of peptidoglycan, allowing for efficient transporter assembly across the entire cell envelope (Mushegian et al., 1996). For example, a specialized LT virB1 homolog (rvhB1) was identified in Rickettsia spp. that encode pathways for biosynthesis and degradation of peptidoglycan; however, virB1 homolog was not identified in N. helminthoeca and other members of the family Anaplasmataceae (Gillespie et al., 2010). Previous electron microscopy showed that only two layers (outer and inner) of membranes and no thickening of the inner or outer leaflet of the outer membrane were present in N. helminthoeca (Rikihisa et al., 1991), suggesting that N. helminthoeca might not possess a peptidoglycan layer.

However, it is possible that N. helminthoeca can still produce precursors or components of peptidoglycan. Since several peptidoglycan components are potent stimulants for innate immunity and anti-microbial responses in host immune defensive cells (Dziarski, 2003; Guan and Mariuzza, 2007; Sukhithasri et al., 2013), the presence of these components in N. helminthoeca could elicit anti-microbial and inflammatory activities in leukocytes, and may account for the high acute mortality of SPD (Philip, 1955; Rikihisa et al., 1991) compared to less severe or chronic infections caused by other Neorickettsia, Ehrlichia, or Anaplasma spp. that lack peptidoglycan biosynthesis genes.

Lipoproteins and putative outer membrane proteins. A previous study indicates that E. chaffeensis expresses mature lipoproteins on the bacterial surface, which induced delayed-type hypersensitivity reaction in dogs (Huang et al., 2008). This study found N. helminthoeca, like other sequenced members of the family Anaplasmataceae, encodes all three lipoprotein-processing enzymes (Lgt, LspA, and Lnt) (Table 13) (Gupta and Wu, 1991; Paetzel et al., 2002). Computational analysis with LipoP 1.0 (http://www.cbs.dtu.dk/services/LipoP) (Juncker et 2003) identified thirteen putative lipoproteins in N. helminthoeca (Table 13), which may also be involved in pathogenesis and immune response in infected canids as in E. chaffeensis (Huang et al., 2008). Homologs of several N. helminthoeca lipoproteins are also identified as lipoproteins in N. risticii, including OmpA, CBS domain protein and VirB6 family proteins (Table 5 and 14) (Lin et al., 2009).

Computational analysis using the pSort-B algorithm predicted only four outer membrane proteins, two of which (BamD lipoprotein and beta-barrel OMP BamA, also called Omp85/YaeT), are part of the beta-barrel assembly machinery (BAM) and essential for the folding and insertion of outer membrane proteins of Gram-negative bacteria (Surana et al., 2004) (Table 4). Unlike Ehrlichia and Anaplasma spp. that encode a diverse members of the OMP-1/P28/MSP2/P44 outer member superfamily proteins (Pfam01617), Neorickettsia spp. encode only one group of putative outer surface proteins that falls into this PFAM family (Dunning Hotopp et al., 2006). This group of proteins consists of three N. helminthoeca surface proteins (NSP1/2/3), which are approximately 30 kDa in mass and likely surface-exposed based on their similarities to Ehrlichia P28/Omp-1 (Ohashi et al., 1998a; Ohashi et al., 2001), A. phagocytophilum P44 (Zhi et al, 1998), and N. risticii/N. sennetsu NSPs (Gibson et al., 2010; Gibson et al., 2011) (FIG. 6B and Table 4).

In addition to NSP family OMPs, several studies have identified additional sets of potential surface proteins in other Neorickettsia spp., which include a 51-kDa protein (P51) and Neorickettsia strain-specific antigens (SSA) (Biswas et al., 1998; Vemulapalli et al., 1998; Rikihisa et al., 2004; Lin et al., 2009; Gibson et al., 2010; Gibson et al., 2011). P51 belongs to an ortholog cluster (cluster 409) that exists in all Rickettsiales (Dunning Hotopp et al., 2006), and is highly conserved among all sequenced Neorickettsia spp. including N. helminthoeca (NHE_RS00965) and the SF agent (Rikihisa et al., 2004) (FIG. 6A). Previous studies have shown that P51 is the major antigenic protein recognized in horses with Potomac horse fever, and an immunofluorescence assay (IFA) using anti-P51 antibody on non-permeabilized N. risticii organisms showed a ring-like labeling pattern surrounding the bacteria, indicating that P51 is a surface-exposed antigen (Gibson and Rikihisa, 2008). P51 of N. sennetsu was demonstrated as a porin (Gibson et al., 2010). Phylogeny estimation (FIG. 6A), SignalP prediction (http://www.cbs.dtu.dk/services/SignalP/), and two-dimensional structures (FIG. 9) suggests that, similar to P51 of N. sennetsu and N. risticii, N. helminthoeca P51 is likely a β-barrel protein localized to the outer membrane.

Strain-specific antigens (SSAs), proteins of ˜50 kDa with extensive intramolecular repeats, have been reported to be a protective antigen of N. risticii against homologous challenge (Biswas et al., 1998; Dutta et al., 1998). Unlike N. risticii or N. sennetsu that encodes two to three tandem genes of nonidentical SSAs, N. helminthoeca only encodes one SSA protein (NHE_RS03855, 35 kDa) (FIG. 6C, Table 4 and 13). Phylogenetic analysis reveals that the SSA family proteins in N. sennetsu and N. risticii likely expanded following divergence from N. helminthoeca, but prior to the divergence of N. risticii and N. sennetsu (FIG. 6C). Sequence analysis also identified several intramolecular tandem repeats in N. helminthoeca P51 and SSA proteins (Table 14), suggesting that they might play important roles in pathogenesis and pathogen-host interactions (Citti and Wise, 1995; Smith et al., 1996).

Immunoreactivities of putative outer membrane proteins. Except for Candidatus“X. pacificiensis” that maintains many genes involved in flagella assembly like hook, ring, and rod (Kwan and Schmidt, 2013), all members of the family Anaplasmataceae lack LPS, capsule, flagella, or common pili (Dunning Hotopp et al., 2006). In agreement with previous electron microscope images (Rikihisa et al., 1991), analysis of N. helminthoeca genome indicates that it did not produce a type 4 pili. Therefore, outer membrane proteins play critical roles in bacterium-host cell interactions and induce strong humoral immune responses (Rikihisa et al., 1992; Rikihisa et al., 1994; Ohashi et al., 1998b; Zhi et al., 1998; Rikihisa et al., 2004; Gibson et al., 2011). Analysis of infection-induced immune reactions to outer membrane proteins provide tools to determine prevalence of N. helminthoeca exposure/infection among various species of animals, and provide novel rapid immunodiagnostic methods and protective vaccines for SPD as disclosed herein.

To elucidate immune reactions of SPD dog sera to P51, NSP1/2/3, and SSA, these proteins were cloned into the pET-33b(+) expression vector, and recombinant proteins were purified from transformed E. coli (FIG. 7A). The immunoreactivities of these surface proteins were analyzed using defined N. helminthoeca IFA-positive dog sera (Rikihisa et al., 1991). Western blot analysis results showed that P51, NSP1/2/3, and SSA proteins were recognized by antisera from NH1 and NH3 dogs experimentally infected with N. helminthoeca by feeding trematodes-parasitized fish and seroconverted (IFA titers of 1:640 and 1:1,280, respectively, using N. helminthoeca-infected DH82 cells as the antigen) (Rikihisa et 1991), with NSP2 and SSA as the strongest sero-reactive antigens (FIGS. 7C-D). In addition, N. helminthoeca-positive dog sera from naturally infected dogs from Southern California recognized P51 and SSA and weakly against NPS3, whereas NSP1 and NSP2 were only detected by “M” sera (FIGS. 7E-F). As a control, antisera from the horse experimentally infected with N. risticii did not react with any of these membrane proteins from N. helminthoeca (FIG. 7B). These data indicate that N. helminthoeca OMPs including P51, SSA, and NSPs can be recognized by the immune system of N. helminthoeca-infected dogs.

A previous study showed that sera from N. helminthoeca-infected dogs, N. sennetsu-infected horse, N. risticii-infected horses, or E. canis-infected dogs cross-reacted with other species but with at least 16-fold lower than those for homologous antigens by immunofluorescence assay (Rikihisa, 1991b; Rikihisa. et al., 1991). This study also showed that approximately 78-80 kDa and 64 kDa proteins were the major antigens shared by N. helminthoeca, N. risticii, N. sennetsu, and E. canis (Rikihisa, 1991b) (FIGS. 7B-D). These cross-reactive antigens were likely more conserved heat-shock proteins or molecular chaperones, and their molecular weights were different from predicted outer membrane proteins of N. helminthoeca analyzed in the current study (from 23 to 51 kDa). Therefore, in current Western blotting with the dilution of sera at 1:400, horse sera against N. risticii recognized none of N. helminthoeca OMPs (FIG. 7B), whereas dog sera against N. helminthoeca only detected proteins at ˜64 and 80-kD from N. risticii (FIGS. 7C-D), showing that these recombinant OMPs can be used for specific diagnosis of N. helminthoeca-infected dogs.

### Conclusion and Discussion

Despite expansion of DNA sequences of Neorickettsia spp. in various trematode species worldwide, biology and natural history have been best studied in N. helminthoeca, the type species of the genus Neorickettsia. In this study, the complete genome sequence of N. helminthoeca was determined and analyzed, providing a valuable resource necessary for understanding the metabolism of N. helminthoeca and its digenean host associations, the evolution and phylogeny among Neorickettsia spp., potential virulence factors of N. helminthoeca, pathogenic mechanisms of SPD, and environmental spreading of N. helminthoeca and trematodes infection in nature. Comparative genomics data of three Neorickettsia spp. of known biological significance is expected to help elucidating biology of other Neorickettsia spp. in the environment.

As SPD progression is rapid, and the case fatality rate is quite high, prevention and early diagnosis of SPD are critical. The serological assay based on defined outer membrane protein antigens is simple, consistent, specific, objective, and convenient, thus helps generating epidemiological information on N. helminthoeca exposure among various wild and domestic animals to raise awareness of SPD. Similar to bats that are the definitive hosts of Acanthatrium oregonense trematodes, the vector of N. risticii transmission (Gibson et al., 2005; Gibson and Rikihisa, 2008), the definitive hosts of N. helminthoeca-infected trematodes in nature are likely asymptomatic, but have antibodies against N. helminthoeca.

Furthermore, these recombinant proteins are used herein in a simple and rapid serodiagnostic test for SPD in dogs. The limitation of the assay is, as in any other serologic assays, false negative results at early stages of infection and in immunosuppressed dogs. Clinical diagnosis is used to determine sensitivity and specificity of the test using a larger number of well-defined canine specimens from broader geographic regions. For this and understanding the pathogenesis and canine immune responses in SPD, culture isolation of additional N. helminthoeca strains is desirable. Characterization of the antigenic surface proteins of N. helminthoeca provides valuable information for the development of rapid, sensitive, and specific serodiagnostic approaches or preventive vaccines for SPD as disclosed herein.

### Experimental Procedures

Organisms Culture, Bacteria Purification, and DNA Preparation.

N. helminthoeca Oregon strain, which was previously isolated from dog NH1 fed with fluke N. salmincola-infested salmon kidneys (Rikihisa et al, 1991), was cultured in DH82 cells from the frozen cell stock in Dulbecco's minimal essential medium supplemented with 10% fetal bovine serum and 2 mM L-glutamine. Cultures were incubated at 37° C. under 5% CO₂ in a humidified atmosphere. To purify host cell-free bacteria for genome sequencing, infected cells (>95% infection) were harvested and Dounce homogenized in SPK buffer (0.2 M sucrose and 0.05 M potassium phosphate, pH 7.4). Lysed cells were centrifuged at 500×g and 700×g to remove unbroken cells and nuclei, filtered through 5.0- and 2.7-μm syringe filters, and centrifuged at 10,000×g to pellet host cell-free bacteria, Genomic DNA was purified using a Genomic-tip 20/G (QIAGEN, Valencia, Calif.) according to manufacturer's instructions, and host DNA contamination was verified to be <0.1% by PCR using specific primers targeting N. helminthoeca 16S rRNA gene and canine G3PDH DNA.

Sequencing and Annotation.

Indexed Illumina mate pair libraries were prepared following the mate pair library v2 sample preparation guide (Illumina, San Diego, Calif.), with two modifications. First, the shearing was performed with the Covaris E210 (Covaris, Wobad, Mass.). The DNA was purified between enzymatic reactions and the size selection of the library was performed with AMPure XT beads (Beckman Coulter Genomics, Danvers, Mass.).

Illumina non-Truseq paired end genomic DNA libraries were constructed using the KAPA library preparation kit (Kapa Biosystems, Woburn, Mass.). DNA was fragmented with the Covaris E210. Then libraries were prepared using a modified version of manufacturer's protocol. The DNA was purified between enzymatic reactions and the size selection of the library was performed with AMPure XT beads (Beckman Coulter Genomics, Danvers, Mass.). For indexed samples the PCR amplification step was performed with primers containing a six nucleotide index sequence.

Concentration and fragment size of libraries were determined using the DNA High Sensitivity Assay on the LabChip GX (Perkin Elmer, Waltham, Mass.) and qPCR using the KAPA Library Quantification Kit (Complete, Universal) (Kapa Biosystems, Woburn, Mass.). The mate pair library was sequenced on an Illumina HiSeq 2500 (Illumina, San Diego, Calif.) while the paired end library was sequenced on an illumina MiSeq (Illumina, San Diego, Calif.).

DNA samples for PacBio sequencing were sheared to 8 khp using the Covaris gTube (Woburn, Mass.). Sequencing libraries were constructed and prepared for sequencing using the DNA Template Prep Kit 2.0 (3 kbp-10 khp) and the DNA/Polymerase Binding Kit 2.0 (Pacific Biosciences. Menlo Park, Calif.). Libraries were loaded onto v2 SMRT Cells, and sequenced with the DNA Sequencing Kit 2.0 (Pacific Biosciences).

Five assemblies were generated with various combinations of the data and assembly algorithms: (1) Celera Assembler v7.0 of only PacBio data, (2) Celera Assembler v7.0 of to PacBio data with correction using Illumina paired end data, (3) HGAP assembly of only PacBio data, (4) MaSuRCA 1.9.2 assembly of Illumina paired end data subsampled to 50× coverage, and (5) MaSuRCA 1.9.2 assembly of Illumina paired end data subsampled to 80× coverage. The first assembly was the optimal assembly, namely the one generated with Cetera Assembler v7.0 with only the PacBio data. The data set was subsampled to ˜22× coverage of the longest reads using an 8 kbp minimum read length cutoff, with the remainder of the reads used for the error correction step. The resulting single-contig assembly totaled ˜89.4 Kbp with 41.68% GC-content. The genome was trimmed to remove overlapping sequences, oriented, circularized, and rotated to the predicted origin of replication. Annotation for this finalized genome assembly was generated using the IGS prokaryotic annotation pipeline (Galens et al., 2011) and deposited in GenBank (accession number NZ_CP007481.1).

Bioinformatic Analysis.

The 16S rRNA, NSP, P51, and SSA proteins were aligned with their Neorickettsia orthologs using CLUSTALW (Thompson et al., 1994) as implemented in BioEdit 7.2.5 (Hall, 1999) resulting in 1522 nt, 326 aa, 516 aa, and 578 aa alignments, respectively. A phylogenetic tree was inferred from the 16S rRNA alignment using RAxML v.7.3.0 (Stamatakis et al., 2005) with the GTRGAMMA model, specifically “RAxMLHPC -f a -m GTRGAMMA -p12345-x12345-N autoMRE -n T20”. The DIRE-based bootstopping criterion was not met, resulting in the use of 1000 bootstraps. For the protein alignments, the best-fit model of amino acid substitution was determined for each alignment separately with ProtTest3.2 (Darriba et al., 2011), with all 15 models of protein evolution tested in addition to the +G parameter. WAG+G was determined to be the best model for NSP and SSA while JTT was determined to be the best model for P51. Phylogenetic trees were inferred from the NSP and SSA alignments using RAxML v.7.3.0 (Stamatakis et al., 2005) with the best model, specifically “RAxMLHPC -f a -m PROTGAMMAWAG -p12345 -x 12345 -N autoMRE -n T20”. The MRE-based bootstopping criterion was met at 350 replicates for NSP and SSA. Phylogenetic trees were inferred from the P51 alignment using RAxML v.7.3.0 (Stamatakis et al., 2005) with the best model, specifically “RAxMLHPC -f a -m PROTCATJTT -p12345 -x12345 -N autoMRE -n T20”. The MRE-based bootstopping criterion was met at 50 replicates for P51. All trees and bootstrap values were visualized in Dendroscope v3.5.7.

The GC-skew was calculated as (C−G)/(C+G) in windows of 500 bp with step size of 250 bp along the chromosome. Synteny plots between Neorickettsia spp. were generated using MUMmer 3 program with default parameters (Delcher et al., 2002). Protein ortholog clusters among Neorickettsia spp., and N. helminthoeca-specific genes compared to other related organisms were determined by using reciprocal BLASTP with cutoff scores of E<10⁻¹⁰.

Metabolic pathways and transporters were compared across genomes using (1) the ortholog clusters generated with reciprocal BLASTP, (2) Genome Properties (Haft et al., 2005), (3) TransportDB (Ren et al., 2007), (4) Kyoto Encyclopedia of Genes and Genomes (KEGG, http://www.kegg.jp), and (5) Biocyc (Krieger et al., 2004). Signal peptides and membrane proteins were predicted using the pSort-B algorithm (http://psort.org/psortb/) (Yu et al., 2010), and lipoproteins were predicted by LipoP 1.0 (http://www.cbs.dtu.dk/services/LipoP) (Juncker et al., 2003).

Cloning, Expression, and Western Blot Analysis of Putative N. helminthoeca Outer Membrane Proteins.

Full-length p51, nsp1/2/3, and ssa genes without the signal peptide sequence were PCR amplified from N. helminthoeca genomic DNA, using specific primers (Table 15) and cloned into the pET-33b(+) vector (Novagen, Billerica). The plasmids were amplified by transformation into Escherichia coli PX5α cells (Protein Express, Inc. Cincinnati, Ohio), and the inserts were confirmed by sequencing. The plasmids were transformed into E. coli BL21(DE3) (Protein Express), and the expression of recombinant proteins was induced with 1 mM isopropyl β-d-thiogalactopyranoside. E. coli was sonicated for a total of 5 min (15 s pulse with 45 s interval) on ice, and the pellet containing recombinant protein was washed with 1% Triton X-100 in sodium phosphate buffer (SPB: 50 mM sodium phosphate, pH 8.0, 0.3 M NaCl). Recombinant proteins were denatured and solubilized with 6 M urea in SPB (for P51, SSA, and NSP2/3), or 6M Guanidine HCl in SPB (for NSP1) at 4° C. for 1 hr. Proteins were purified on a HisPur Cobalt Affinity resin (Pierce, Rockford, Ill.) and dialyzed using Buffer A (50 mM KCl, 100 mM NaCl, 50 mM Tris-HCl, pH 8.0) containing decreasing concentrations of urea (3 M, 1 M, then 0 M). Protein concentrations were determined by BCA assay (Pierce).

Bacterial lysates of purified N. risticii or N. helminthoeca, and recombinant NSP1/2/3, SSA, and P51 were subjected to SDS-polyacrylamide gel electrophoresis (SDS-PAGE) and Western blot analysis as described previously (Lin et al., 2002). Gels were stained using GelCode Blue (Pierce), and the immuno-reactivities of these recombinant proteins were determined by western blot analysis using SPD dog sera against N. helminthoeca or horse anti-N. risticii serum as a negative control at 1:400 dilutions. Defined SPD dog sera against N. helminthoeca were obtained from dogs orally fed by fluke N. salmincola-infested salmon kidneys infected with N. helminthoeca, and sera collected at day 13 and 15 post exposure with IFA titers at 1:640 (NH1) and 1:1,280 (NH3), respectively (Rikihisa. et al., 1991). Clinical dog sera tested positive for N. helminthoeca-infection were received from southern California (“M” sera—IFA titer 1:80, from Dana Point, Calif. In 2012; “D” sera—PCR-positive for N. helminthoeca 16S rRNA gene, from Aliso Viejo, Calif. In 2010). Horse anti-N. risticii serum (Pony 19) was collected from a pony inoculated intravenously with N. risticii-infected U-937 cells (IFA titer 1:640) (Rikihisa et al., 1988). Reacting bands were detected with Horseradish peroxidase (HRP)-conjugated goat anti-dog (KPL Gaithersburg, Md.) or anti-horse (Jackson Immuno Research, West Grove, Pa.) secondary antibodies, and visualized with enhanced chemiluminescence (ECL) by incubating the membranes with LumiGLO™ chemiluminescent reagent (Pierce). Images were captured using an LAS3000 image documentation system (FUJIFILM Medical Systems USA, Stamford, Conn.).

GenBank Accession Numbers and Abbreviations of Bacteria.

N. helminthoeca Oregon (NHO), NZ_CP007481.1 (this example); N. risticii Illinois (NRI), NC_013009.1; N. sennetsu Miyayama (NSE), NC_007798.1; A. phagocytophilum HZ (APH), NC_007797.1; A. marginale Florida (AMA), NC_012026.1; E. chaffeensis Arkansas (ECH), NC_007799.1; E. canis Jake (ECA), NC_007354.1; E. ruminantium Welgevonden (ERU), NC_005295.2; E. muris AS145 (EMU), NC_023063.1; Ehrlichia sp. HF (EHF), NZ_CP007474.1; Wolbachia pipientis (wMel, Wolbachia endosymbiont of Drosophila melanoga), NC_002978.6; Wolbachia endosymbiont of Brugia malayi (wBm), NC_006833.1; Neorickettsia endobacterium of Fasciola hepatica (NFh), NZ _LNGI00000000, Candidatus Xenolissoclinum pacificiensis L6, AXCJ00000000.

### REFERENCES

Anderson, B. E., Greene, C. E., Jones, D. C., and Dawson, J. E. (1992) Ehrlichia ewingii sp. nov., the etiologic agent of canine granulocytic ehrlichiosis. Int J Syst Bacteriol 42: 299-302.
Backert, S., and Meyer, T. F. (2006) Type IV secretion systems and their effectors in bacterial pathogenesis. Curr Opin Microbiol 9: 207-217.
Bagos, P. G., Liakopoulos, T. D., Spyropoulos, I. C., and Hamodrakas, S. J. (2004) PRED-TMBB: a web server for predicting the topology of beta-barrel outer membrane proteins. Nucleic Acids Res 32: W400-404.
Bagos, P. G., Nikolaou, E. P., Liakopoulos, T. D., and Tsirigos, K. D. (2010) Combined prediction of Tat and Sec signal peptides with hidden Markov models. Bioinformatics 26: 2811-2817.
Barnewall, R. E., and Rikihisa, Y. (1994) Abrogation of gamma interferon-induced inhibition of Ehrlichia chaffeensis infection in human monocytes with iron-transferrin. Infect Immun 62: 4804-4810.
Barnewall, R. E., Ohashi, N., and Rikihisa, Y. (1999) Ehrlichia chaffeensis and E. sennetsu, but not the human granulocytic ehrlichiosis agent, colocalize with transferrin receptor and up-regulate transferrin receptor mRNA by activating iron-responsive protein 1. Infect Immun 67: 2258-2265.
Bendtsen, J. D., Nielsen, H., Widdick, D., Palmer, T., and Brunak, S. (2005) Prediction of twin-arginine signal peptides. BMC Bioinformatics 6: 167.
Bennett, V., and Baines, A. J. (2001) Spectrin and ankyrin-based pathways: metazoan inventions for integrating cells into tissues. Physiol Rev 81: 1353-1392.
Bennington, E., and Pratt, I. (1960) The life history of the salmonpoisoning fluke, Nanophyetus salmincola (Chapin). J Parasitol 46: 91-100.
Bingle, L. E., Bailey, C. M., and Pallen, M. J. (2008) Type VI secretion: a beginner's guide. Curr Opin Microbiol 11: 3-8.
Biswas, B., Vemulapalli, R, and Dutta, S. K. (1998) Molecular basis for antigenic variation of a protective strain-specific antigen of Ehrlichia risticii. Infect Immnun 66: 3682-3688.
Booth, A. J., Stogdale, L., and Grigor, J. A. (1984) Salmon poisoning disease in dogs on southern vancouver island. Can Vet J 25: 2-6.
Brownlie, J. C., Cass, B. N., Riegler, M., Witsenburg, J. J., Iturbe-Ormaetxe, I., McGraw, E. A., and O'Neill, S. L. (2009) Evidence for metabolic provisioning by a common invertebrate endosymbiont, Wolbachia pipientis, during periods of nutritional stress. PLoS Pathog 5: e1000368.
Cascales, E., and Christie, P. J. (2003) The versatile bacterial type IV secretion systems. Nat Rev Microbiol 1: 137-149.
Cheng, Z., Wang, X., and Rikihisa., Y. (2008) Regulation of type IV secretion apparatus genes during Ehrlichia chaffeensis intracellular development by a previously unidentified protein. J Bacteriol 190: 2096-2105.
Cheng, Z., Lin, M., and Rikihisa, Y. (2014) Ehrlichia chaffeensis proliferation begins with NtrY/NtrX and PutA/GlnA upregulation and CtrA degradation induced by proline and glutamine uptake. MBio 5: e02141.
Cheng, Z., Kumagai, Y., Lin, M., Zhang, C., and Rikihisa, Y. (2006) Intra-leukocyte expression of two-component systems in Ehrlichia chaffeensis and Anaplasma phagocytophilum and effects of the histidine kinase inhibitor closantel. Cell Microbiol 8: 1241-1252.
Cheng, Z., Miura, K., Popov, V. L., Kumagai, Y., and Rikihisa, Y. (2011) Insights into the CtrA regulon in development of stress resistance in obligatory intracellular pathogen Ehrlichia chaffeensis. Mol Microbiol 82: 1217-1234.
Christie, P. J., Whitaker, N., and Gonzalez-Rivera, C. (2014) Mechanism and structure of the bacterial type IV secretion systems. Biochim Biophys Acta 1843: 1578-1591.
Cianciotto, N. P. (2005) Type II secretion: a protein secretion system for all seasons. Trends Microbiol 13: 581-588.
Citti, C., and Wise, K. S. (1995) Mycoplasma hyorhinis vlp gene transcription: critical role in phase variation and expression of surface lipoproteins. Mol Microbiol 18: 649-660.
Cordes, D. O., Perry, B. D., Rikihisa, Y., and Chickering, W. R. (1986) Enterocolitis caused by Ehrlichia sp. in the horse (Potomac horse fever). Vet Pathol 23: 471-477.
Cordy, D. R., and Gorham, J. R. (1950) The pathology and etiology of salmon disease. Am J Pathol 26: 617-637.
Costa, T. R., Felisberto-Rodrigues, C., Meir, A., Prevost, M. S., Redzej, A., Trokter, M., and Waksman, G. (2015) Secretion systems in Gram-negative bacteria: structural and mechanistic insights. Nat Rev Microbiol 13: 343-359.
Crossman, L. (2006) Budget genome. Nat Rev Microbiol 4: 326-327.
Darriba, D., Taboada, G. L., Doallo, R., and Posada, D. (2011) ProtTest 3: fast selection of best-fit models of protein evolution. Bioinformatics 27: 1164-1165.
Delcher, A. L., Phillippy, A., Carlton, J., and Salzberg, S. L. (2002) Fast algorithms for large-scale genome alignment and comparison. Nucleic Acids Res 30: 2478-2483.
Delepelaire, P. (2004) Type I secretion in gram-negative bacteria. Biochim Biophys Acta 1694: 149-161.
Dorman, C. J., McKenna, S., and Beloin, C. (2001) Regulation of virulence gene expression in Shigella flexneri, a facultative intracellular pathogen. Int J Med Microbiol 291: 89-96.
Dumler, J. S., Barbet, A. F., Bekker, C. P., Dasch, G. A., Palmer, G. H., Ray, S. C. et al. (2001) Reorganization of genera in the families Rickettsiaceae and Anaplasmataceae in the order Rickettsiales: unification of some species of Ehrlichia with Anaplasma, Cowdria with Ehrlichia and Ehrlichia with Neorickettsia, descriptions of six new species combinations and designation of Ehrlichia equi and ‘HGE agent’ as subjective synonyms of Ehrlichia phagocytophilum. Int J Syst Evol Microbiol 51: 2145-2165.
Dunning Hotopp, J. C., Lin, M., Madupu, R., Crabtree, J., Angiuoli, S. V., Eisen, J. A. et al. (2006) Comparative genomics of emerging human ehrlichiosis agents. PLoS Genet 2: e21.
Dutta, S. K., Vemulapalli, R., and Biswas, B. (1998) Association of deficiency in antibody response to vaccine and heterogeneity of Ehrlichia risticii strains with Potomac horse fever vaccine failure in horses. J Clin Microbiol 36: 506-512.
Dutta, S. K., Penney, B. E., Myrup, A. C., Robl, M. G., and Rice, R. M. (1988) Disease features in horses with induced equine monocytic ehrlichiosis (Potomac horse fever) Am J Vet Res 49: 1747-1751.
Dziarski, R. (2003) Recognition of bacterial peptidoglycan by the innate immune system. Cell Mol Life Sci 60: 1793-1804.
Foreyt, W. J., Gorham, J. R., Green, J. S., Leathers, C. W., and LeaMaster, B. R. (1987) Salmon poisoning disease in juvenile coyotes: clinical evaluation and infectivity of metacercariae and rickettsiae. J Wildl Dis 23: 412-417.
Foster, J., Ganatra, M., Kamal, I., Ware, J., Makarova, K., Ivanova, N. et al. (2005) The Wolbachia genome of Brugia malayi: endosymbiont evolution within a human pathogenic nematode. PLoS Biol 3: e121.
Frutos, R., Viari, A., Vachiery, N., Boyer, F., and Martinez, D. (2007) Ehrlichia ruminantium: genomic and evolutionary features. Trends Parasitol 23: 414-419.
Galens, K., Orvis, J., Daugherty, S., Creasy, H. H., Angiuoli, S., White, O. et al. (2011) The IGS Standard Operating Procedure for Automated Prokaryotic Annotation. Stand Genomic Sci 4: 244-251.
Gao, R., Mack, T. R., and Stock, A. M. (2007) Bacterial response regulators: versatile regulatory strategies from common domains. Trends Biochem Sci 32: 225-234.
Garcia-Garcia, J. C., Rennoll-Bankert, K. E., Pelly, S., Milstone, A. M., and Dumler, J. S. (2009) Silencing of host cell CYBB gene expression by the nuclear effector AnkA of the intracellular pathogen Anaplasma phagocytophilum. Infect Immun 77: 2385-2391.
Gibson, K., Kumagai, Y., and Rikihisa, Y. (2010) Proteomic analysis of Neorickettsia sennetsu surface-exposed proteins and porin activity of the major surface protein P51. J Bacteriol 192: 5898-5905.
Gibson, K. E., and Rikihisa, Y. (2008) Molecular link of different stages of the trematode host of Neorickettsia risticii to Acanthatrium oregonense. Environ Microbiol 10: 2064-2073.
Gibson, K. E., Rikihisa, Y., Zhang, C., and Martin, C. (2005) Neorickettsia risticii is vertically transmitted in the trematode Acanthatrium oregonense and horizontally transmitted to bats. Environ Microbiol 7: 203-212.
Gibson, K. E., Pastenkos, G., Moesta, S., and Rikihisa, Y. (2011) Neorickettsia risticii surface-exposed proteins: proteomics identification, recognition by naturally-infected horses, and strain variations. Vet Res 42: 71.
Gillespie, J. J., Brayton, K. A., Williams, K. P., Diaz, M. A., Brown, W. C., Azad, A. F., and Sobral, B. W. (2010) Phylogenomics reveals a diverse Rickettsiales type IV secretion system. Infect Immun 78: 1809-1823.
Gillespie, J. J., Kaur, S. J., Rahman, M. S., Rennoll-Bankert, K., Sears, K. T., Beier-Sexton, M., and Azad, A. F. (2015) Secretome of obligate intracellular Rickettsia. FEMS Microbiol Rev 39: 47-80.
Gillespie, J. J., Ammerman, N. C., Dreher-Lesnick, S. M., Rahman, M. S., Worley, M. J., Setubal, J. C. et al. (2009) An anomalous type IV secretion system in Rickettsia is evolutionarily conserved. PLoS One 4: e4833.
Greiman, S. E., Tkach, V. V., Pulis, E., Fayton, T. J., and Curran, S. S. (2014) Large scale screening of digeneans for Neorickettsia endosymbionts using real-time PCR reveals new Neorickettsia genotypes, host associations and geographic records. PLoS One 9: e98453.
Greiman, S. E., Rikihisa, Y., Cain, J., Vaughan, J. A., and Tkach, V. V. (2016) Germs within Worms: Localization of Neorickettsia sp. within Life Cycle Stages of the Digenean Plagiorchis elegans. Appl Environ Microbiol 82: 2356-2362.
Greiman, S. E., Vaughan, J. A., Elmahy, R., Adisakwattana, P., Van Ha, N., Fayton, T. J. et al. (2017) Real-time PCR detection and phylogenetic relationships of Neorickettsia spp. in digeneans from Egypt, Philippines, Thailand, Vietnam and the United States. Parasitol Int 66: 1003-1007.
Guan, R., and Mariuzza, R. A. (2007) Peptidoglycan recognition proteins of the innate immune system. Trends Microbiol 15: 127-134.
Gupta, S. D., and Wu, H. C. (1991) Identification and subcellular localization of apolipoprotein N-acyltransferase in Escherichia coli. FEMS Microbiol Lett 62: 37-41.
Haft, D. H., Selengut, J. D., Brinkac, L. M., Zafar, N., and White, O. (2005) Genome Properties: a system for the investigation of prokaryotic genetic content for microbiology, genome annotation and comparative genomics. Bioinformatics 21: 293-306.
Hall, T. A. (1999) BioEdit: a user-friendly biological sequence alignment editor and analysis program for Windows 95/98/NT. Nucleic Acids Symposium Series 41: 95-98.
Headley, S. A., Scorpio, D. G., Vidotto, O., and Dumler, J. S. (2011) Neorickettsia helminthoeca and salmon poisoning disease: a review. Vet J 187: 165-173.
Headley, S. A., Vidotto, O., Scorpio, D., Dumler, J. S., and Mankowski, J. (2004) Suspected cases of Neorickettsia-like organisms in Brazilian dogs. Ann NY Acad Sci 1026: 79-83.
Headley, S. A., Scorpio, D. G., Barat, N. C., Vidotto, O., and Dumler, J. S. (2006) Neorickettsia helminthoeca in dog, Brazil. Emerg Infect Dis 12: 1303-1304.
Henderson, I. R., Navarro-Garcia, F., Desvaux, M., Fernandez, R. C., and Ala'Aldeen, D. (2004) Type V protein secretion pathway: the autotransporter story. Microbiol Mol Biol Rev 68: 692-744.
Huang, H., Wang, X., Kikuchi, T., Kumagai, Y., and Rikihisa, Y. (2007) Porin activity of Anaplasma phagocytophilum outer membrane fraction and purified P44. J Bacteriol 189: 1998-2006.
Huang, H., Un, M., Wang, X., Kikuchi, T., Mottaz, H., Norbeck, A., and Rikihisa, Y. (2008) Proteomic analysis of and immune responses to Ehrlichia chaffeensis lipoproteins. Infect Immun 76: 3405-3414.
IJdo, J. W., Carlson, A. C., and Kennedy, E. L. (2007) Anaplasma phagocytophilum AnkA is tyrosine phosphorylated at EPIYA motifs and recruits SHP-1 during early infection. Cell Microbiol 9: 1284-1296.
Ioannidis, P., Dunning Hotopp, J. C., Sapountzis, P., Siozios, S., Tsiamis, G., Bordenstein, S. R. et al, (2007) New criteria for selecting the origin of DNA replication in Wolbachia and closely related bacteria. BMC Genomics 8: 182.
Jernigan, K. K., and Bordenstein, S. R. (2014) Ankyrin domains across the Tree of Life. Peer J 2: e264.
Jorda, J., and Kajava, A. V. (2009) T-REKS: identification of Tandem REpeats in sequences with a K-meanS based algorithm. Bioinformatics 25: 2632-2638.
Juncker, A. S., Willenbrock, H., Von Heijne, G., Brunak, S., Nielsen, H., and Krogh, A. (2003) Prediction of lipoprotein signal peptides in Gram-negative bacteria. Protein Sci 12: 1652-1662.
Kremer, N., Voronin, D., Charif, D., Mavingui, P., Mollereau, B., and Vavre, F. (2009) Wolbachia interferes with ferritin expression and iron metabolism in insects. PLoS Pathog 5: e1000630.
Krieger, C. J., Zhang, P., Mueller, L. A., Wang, A., Paley, S., Arnaud, M. et al. (2004) MetaCyc: a multiorganism database of metabolic pathways and enzymes. Nucleic Acids Res 32: D438-442.
Kumagai, Y., Huang, H., and Rikihisa, Y. (2008) Expression and porin activity of P28 and OMP-1F during intracellular Ehrlichia chaffeensis development. J Bacteriol 190: 3597-3605.
Kumagai, Y., Cheng, Z., Lin, M., and Rikihisa, Y. (2006) Biochemical activities of three pairs of Ehrlichia chaffeensis two-component regulatory system proteins involved in inhibition of lysosomal fusion. Infect Immun 74: 5014-5022.
Kumagai, Y., Matsuo, J., Cheng, Z., Hayakawa, Y., and Rikihisa, Y. (2011) Cyclic dimeric GMP signaling regulates intracellular aggregation, sessility, and growth of Ehrlichia chaffeensis. Infection and Immunity 79: 3905-3912.
Kwan, J. C., and Schmidt, E. W. (2013) Bacterial endosymbiosis in a chordate host: long-term co-evolution and conservation of secondary metabolism. PLoS One 8: e80822.
Lai, T. H., Kumagai, Y., Hyodo, M., Hayakawa, Y., and Rikihisa, Y. (2009) The Anaplasma phagocytophilum PleC histidine kinase and PleD diguanylate cyclase two-component system and role of cyclic Di-GMP in host cell infection. J Bacteriol 191: 693-700.
Lee, P. A., Tullman-Ercek, D., and Georgiou, G. (2006) The bacterial twin-arginine translocation pathway. Annu Rev Microbiol 60: 373-395.
Lee, Z. M., Bussema, C., 3rd, and Schmidt, T. M. (2009) rrnDB: documenting the number of rRNA and tRNA genes in bacteria and archaea. Nucleic Acids Res 37: D489-493.
Lin, M., and Rikihisa, Y. (2003) Ehrlichia chaffeensis and Anaplasma phagocytophilum lack genes for lipid A biosynthesis and incorporate cholesterol for their survival. Infect Immun 71: 5324-5331.

Lin, M., Zhu, M. X., and Rikihisa, Y. (2002) Rapid activation of protein tyrosine kinase and phospholipase C-gamma2 and increase in cytosolic free calcium are required by Ehrlichia chaffeensis for internalization and growth in THP-1 cells. Infect Human 70: 889-898.

Lin, M., den Dulk-Ras, A., Hooykaas, P. J., and Rikihisa, Y. (2007) Anaplasma phagocytophilum AnkA secreted by type IV secretion system is tyrosine phosphorylated by Abl-1 to facilitate infection. Cell Microbiol 9: 2644-2657.
Lin, M., Zhang, C., Gibson, K., and Rikihisa, Y. (2009) Analysis of complete genome sequence of Neorickettsia risticii: causative agent of Potomac horse fever. Nucleic Acids Res 37: 6076-6091.
Lin, M., Liu, H., Xiong, Q., Niu, H., Cheng, Z., Yamamoto, A., and Rikihisa, Y. (2016) Ehrlichia secretes Etf-1 to induce autophagy and capture nutrients for its growth through RAB5 and class III phosphatidylinositol 3-kinase. Autophagy 12: 2145-2166.
Lin, Q., Zhang, C., and Rikihisa, Y. (2006) Analysis of involvement of the RecF pathway in p44 recombination in Anaplasma phagocytophilum and in Escherichia coli by using a plasmid carrying the p44 expression and p44 donor loci. Infect Immun 74: 2052-2062.
Lin, Q., Rikihisa, Y., Felek, S., Wang, X., Massung, R. F., and Woldehiwet, Z. (2004) Anaplasma phagocytophilum has a functional msp2 gene that is distinct from p44. Infect Immun 72: 3883-3889.
Liu, H., Bao, W., Lin, M., Niu, H., and Rikihisa., Y. (2012) Ehrlichia type IV secretion effector ECH0825 is translocated to mitochondria and curbs ROS and apoptosis by upregulating host MnSOD. Cell Microbiol 14: 1037-1050.
Massung, R. F., Lee, K., Mauel, M., and Gusa, A. (2002) Characterization of the rRNA genes of Ehrlichia cheffeensis and Anaplasma phagocytophila. DNA Cell Biol 21: 587-596.
McNulty, S. N., Tort, J. F., Rinaldi, G., Fischer, K., Rosa, B. A., Smircich, P. et al. (2017) Genomes of Fasciola hepatica from the Americas Reveal Colonization with Neorickettsia Endobacteria Related to the Agents of Potomac Horse and Human Sennetsu Fevers. PLoS Genet 13: el 006537.
Millemann, R. E., and Knapp, S. E. (1970) Biology of Nanophyetus salmincola and “salmon poisoning” disease. Adv Parasitol 8: 1-41.
Mitrophanov, A. Y., and Groisman, E. A. (2008) Signal integration in bacterial two-component regulatory systems. Genes Dev 22: 2601-2611.
Mosavi, L. K., Cammett, T. J., Desrosiers, D. C., and Peng, Z. Y. (2004) The ankyrin repeat as molecular architecture for protein recognition. Protein Sci 13: 1435-1448.
Mushegian, A. R., Fuliner, K. J., Koonin, E. V., and Nester, E. W. (1996) A family of lysozyme-like virulence factors in bacterial pathogens of plants and animals. Proc Natl Acad Sci USA 93: 7321-7326.
Natale, P., Bruser, T., and. Driessen, A. J. (2008) Sec- and Tat-mediated protein secretion across the bacterial cytoplasmic membrane—distinct translocases and mechanisms. Biochim Biophys Acta 1778: 1735-1756.
Nene, V., and Kole, C. (2009) Genome mapping and genomics in animal-associated microbes. Berlin: Springer.
Nikaido, H. (2003) Molecular basis of bacterial outer membrane permeability revisited. Microbiol Mol Biol Rev 67: 593-656.
Niu, H., Rikihisa, Y., Yamaguchi, M., and Ohashi, N. (2006) Differential expression of VirB9 and VirB6 during the life cycle of Anaplasma phagocytophilum in human leucocytes is associated with differential binding and avoidance of lysosome pathway. Cell Microbiol 8: 523-534.
Niu, H., Kozjak-Pavlovic, V., Rudel, T., and Rikihisa, Y. (2010) Anaplasma phagocytophilum Ats-1 is imported into host cell mitochondria and interferes with apoptosis induction. PLoS Pathog 6: e1000774.
Niu, H., Xiong, Q., Yamamoto, A., Hayashi-Nishino, M., and Rikihisa, Y. (2012) Autophagosomes induced by a bacterial Beclin 1 binding protein facilitate obligatory intracellular infection. Proc Natl Acad Sci USA 109: 20800-20807.
Ohashi, N., Rikihisa, Y., and Unver, A. (2001) Analysis of transcriptionally active gene clusters of major outer membrane protein multigene family in Ehrlichia canis and E. chaffeensis. Infect Immun 69: 2083-2091.
Ohashi, N., Unver, A., Zhi, N., and Rikihisa, Y. (1998a) Cloning and characterization of multigenes encoding the immunodominant 30-kilodalton major outer membrane proteins of Ehrlichia canis and application of the recombinant protein for serodiagnosis. J Clin Microbiol 36: 2671-2680.
Ohashi, N., Zhi, N., Zhang, Y., and Rikihisa, Y. (1998b) immunodominant major outer membrane proteins of Ehrlichia chaffeensis are encoded by a polymorphic multigene family. Infect Immun 66: 132-139.
Paetzel, M., Karla, A., Strynadka N. C., and Dalbey, R. E. (2002) Signal peptidases. Chem Rev 102: 4549-4580.
Papanikou, E., Karamanou, S., and Economou, A. (2007) Bacterial protein secretion through the translocase nanomachine. Nat Rev Microbiol 5: 839-851.
Park, J., and Rikihisa, Y. (1992) L-arginine-dependent killing of intracellular Ehrlichia risticii by macrophages treated with gamma interferon. Infect Immun 60: 3504-3508.
Philip, C. B. (1955) There's always something new under the parasitological sun (the unique story of helminth-borne salmon poisoning disease). J Parasitol 41: 125-148.
Philip, C. B., Hadlow, W. J., and Hughes, L. E. (1954a) Studies on salmon poisoning disease of canines. I. The rickettsial relationships and pathogenicity of Neorickettsia helminthoeca. Exp Parasitol 3: 336-350.
Philip C. B., Hughes, L. E., Locker, B., and Hadlow, W. J. (1954b) Salmon poisoning disease of canines. II. Further observations on etiologic agent. Proc Soc Exp Biol Med 87: 397-400.
Pretzman, C., Ralph, D., Stothard, D. R., Fuerst, P. A., and Rikihisa, Y. (1995) 16S rRNA gene sequence of Neorickettsia helminthoeca and its phylogenetic alignment with members of the genus Ehrlichia. Int J Syst Bacteriol 45: 207-211.
Ren, Q., and Paulsen, I. T. (2007) Large-scale comparative genomic analyses of cytoplasmic membrane transport systems in prokaryotes. J Mol Microbiol Biotechnol 12: 165-179.
Ren, Q., Chen, K., and Paulsen, I. T. (2007) TransportDB: a comprehensive database resource for cytoplasmic membrane transport systems and outer membrane channels. Nucleic Acids Res 35: 1)274-279.
Rikihisa, Y. (1991a) The tribe Ehrlichieae and ehrlichial diseases. Clin Microbiol Rev 4: 286-308.
Rikihisa, Y. (1991b) Cross-reacting antigens between Neorickettsia helminthoeca and Ehrlichia species, shown by immunofluorescence and Western immunoblotting. J Clin Microbiol 29: 2024-2029.
Rikihisa, Y. (2010) Anaplasma phagocytophilum and Ehrlichia chaffeensis: subversive manipulators of host cells. Nat Rev Microbiol 8: 328-339.
Rikihisa, Y., Stills, H., and Zimmerman, G. (1991) Isolation and continuous culture of Neorickettsia helminthoeca in a macrophage cell line. J Clin Microbiol 29: 1928-1933.
Rikihisa, Y., Ewing, S. A., and. Fox, J. C. (1994) Western immunoblot analysis of Ehrlichia chaffeensis, E. canis, or E. ewingii infections in dogs and humans. J Clin Microbiol 32: 2107-2112.
Rikihisa, Y., Dumler, J. S., and Dasch, G. C. (2005) Neorickettsia. In The Proteobacteria, Part C, Bergey's Manual of Systematic Bacteriology, 2nd Ed. Brenner, K., Staley and Garrity (ed). New York, N.Y.: Springer, pp. 132-137.
Rikihisa, Y., Pretzman, C. I., Johnson, G. C., Reed, S. M., Yamamoto, S., and Andrews, F. (1988) Clinical, histopathological, and immunological responses of ponies to Ehrlichia sennetsu and subsequent Ehrlichia risticii challenge. Infect Immun 56: 2960-2966.
Rikihisa, Y., Ewing, S. A., Fox, J. C., Siregar, A. G., Pasaribu, F. H., and Malole, M. B. (1992) Analyses of Ehrlichia canis and a canine granulocytic Ehrlichia infection. J Clin Microbiol 30: 143-148.
Rikihisa, Y., Zhang, C., Kanter, M., Cheng, Z., Ohashi, N., and Fukuda, T. (2004) Analysis of p51, groESL, and the major antigen P51 in various species of Neorickettsia, an obligatory intracellular bacterium that infects trematodes and mammals. J Clin Microbiol 42: 3823-3826.
Rikihisa, Y., Kawahara, M., Wen, B., Kociba, G., Fuerst, P., Kawamori, F. et al. (1997) Western immunoblot analysis of Haemobartonella muris and comparison of 16S rRNA gene sequences of H. muris, H. felis, and Eperythrozoon suis. J Clin Microbiol 35: 823-829.
Romling, U. (2009) Cyclic Di-GMP (c-Di-GMP) goes into host cells—c-Di-GMP signaling in the obligate intracellular pathogen Anaplasma phagocytophilum. J Bacteriol 191: 683-686.
Sargent, F., Berks, B. C., and Palmer, T. (2006) Pathfinders and trailblazers: a prokaryotic targeting system for transport of folded proteins. FEMS Microbiol Lett 254: 198-207.
Schlegel, M. W., Knapp, S. E., and Millemann, R. E. (1968) “Salmon poisoning” disease. V. Definitive hosts of the trematode vector, Nanophyetus salmincola. J Parasitol 54: 770-774.
Skaar, E. P. (2010) The battle for iron between bacterial pathogens and their vertebrate hosts. PLoS Pathog 6: e1000949.
Smith, G. A., Theriot, J. A., and Portnoy, D. A. (1996) The tandem repeat domain in the Listeria monocytogenes ActA protein controls the rate of actin-based motility, the percentage of moving bacteria, and the localization of vasodilator-stimulated phosphoprotein and profilin. J Cell Biol 135: 647-660.
Stamatakis, A., Ludwig, T., and Meier, H. (2005) RAxML-III: a fast program for maximum likelihood-based inference of large phylogenetic trees. Bioinformatics 21: 456-463.
Sukhithasri, V., Nisha, N., Biswas, L., Anil Kumar, V., and Biswas, R. (2013) Innate immune recognition of microbiol cell wall components and microbiol strategies to evade such recognitions. Microbiol Res 168: 396-406.
Surana, N. K., Grass, S., Hardy, G. G., Li, H., Thanassi, D. G., and Geme, J. W., 3rd (2004) Evidence for conservation of architecture and physical properties of Omp85-like proteins throughout evolution. Proc Natl Acad Sci USA 101: 14497-14502.
Thompson, J. D., Higgins, D. G., and Gibson, T. J. (1994) CLUSTAL W: improving the sensitivity of progressive multiple sequence alignment through sequence weighting, position-specific gap penalties and weight matrix choice. Nucleic Acids Res 22: 4673-4680.
Tischler, A. D., and Camilli A. (2004) Cyclic diguanylate (c-di-GMP) regulates Vibrio cholerae biofilm formation. Mol Microbiol 53: 857-869.
Tkach, V. V., Schroeder, J. A., Greiman, S. E., and Vaughan, J. A. (2012) New genetic lineages, host associations and circulation pathways of Neorickettsia endosymbionts of digeneans. Acta Parasitol 57: 285-292.
Ulrich, L. E., and Zhulin, I. B. (2007) MiST: a microbiol signal transduction database. Nucleic Acids Res 35: D386-390.
Ulrich, L. E., and Zhulin, I. B. (2010) The MiST2 database: a comprehensive genomics resource on microbiol signal transduction. Nucleic Acids Res 38: D401-407.
Ulrich, L. E., Koonin, E. V., and Zhulin, I. B. (2005) One-component systems dominate signal transduction in prokaryotes. Trends Microbiol 13: 52-56.
Vemulapalli, R., Biswas, B., and Dutta, S. K. (1998) Cloning and molecular analysis of genes encoding two immunodominant antigens of Ehrlichia risticii. Microb Pathog 24: 361-372.
Veterinary Practice News (2009). Salmon Poisoning Disease Found In Southern California, URL http://www.veterinarypracticenews.cotn/April-2009/Salmon-Poisoning-Disease-Found-In-Southern-California/
Vollmer, W., and Bertsche, U. (2008) Murein (peptidoglycan) structure, architecture and biosynthesis in Escherichia coli. Biochim Biophys Acta 1778:1714-1734.
Wakeel, A., den Dulk-Ras, A., Hooykaas, P. J., and McBride, J. W. (2011) Ehrlichia chaffeensis tandem repeat proteins and Ank200 are type 1 secretion system substrates related to the repeats-in-toxin exoprotein family. Front Cell Infect Microbiol 1: 22.
Wang, X., Kikuchi, T., and Rikihisa, Y. (2007a) Proteomic identification of a novel Anaplasma phagocytophilum DNA binding protein that regulates a putative transcription factor. J Bacteriol 189: 4880-4886.
Wang, X., Cheng, Z., Zhang, C., Kikuchi, T., and Rikihisa, Y. (2007b) Anaplasma phagocylophilum p44 mRNA expression is differentially regulated in mammalian and tick host cells: involvement of the DNA binding protein ApxR. J Bacteriol 189: 8651-8659.
Ward, N. L., Steven, B., Penn, K., Methe, B. A., and Detrick, W. H., 3rd (2009) Characterization of the intestinal microbiota of two Antarctic notothenioid fish species. Extremophiles 13: 679-685.
Weiss, E., Williams, J. C., Dasch, G. A., and Kang, Y. H. (1989) Energy metabolism of monocytic Ehrlichia. Proc Natl Acad Sci USA 86: 1674-1678.
Wen, B., Rikihisa, Y., Yamamoto, S., Kawabata, N., and Fuerst, P. A. (1996) Characterization of the SF agent, an Ehrlichia sp. isolated from the fluke Stellantchasinus falcatus, by 16S rRNA base sequence, serological, and morphological analyses. Int J Syst Bacteriol 46: 149-154.
Wen, B., Rikihisa, Y., Mott, J., Fuerst, P. A., Kawahara, M., and Suto, C. (1995) Ehrlichia muris sp. nov., identified on the basis of 16S rRNA base sequences and serological, morphological, and biological characteristics. Int J Syst Bacteriol 45: 250-254.
Winkler, H. H. (1976) Rickettsial permeability. An ADP-ATP transport system. J Biol Chem 251: 389-396.
Wu, M., Sun, L. V., Vamathevan, J., Riegler, M., Deboy, R., Brownlie, J. C. et al. (2004) Phylogenomics of the reproductive parasite Wolbachia pipientis wMel: a streamlined genome overrun by mobile genetic elements. PLoS Biol 2: E69.
Wuichet, K., Cantwell, B. J., and Zhulin, I. B. (2010) Evolution and phyletic distribution of two-component signal transduction systems. Curr Opin Microbiol 13: 219-225.
Yu, N. Y., Wagner, J. R., Laird, M. R., Melli, G., Rey, S., Lo, R. et al. (2010) PSORTb 3.0: improved protein subcellular localization prediction with refined localization subcategories and predictive capabilities for all prokaryotes. Bioinformatics 26: 1608-1615.
Zhi, N., Ohashi, N. Rikihisa, Y., Horowitz, H. W., Wormser, G. P., and Hechemy, K. (1998) Cloning and expression of the 44-kilodalton major outer membrane protein gene of the human granulocytic ehrlichiosis agent and application of the recombinant protein to serodiagnosis. J Clin Microbiol 36: 1666-1673.
Zhu, B., Nethery, K. A., Kuriakose, J. A., Wakeel, A., Zhang, X., and McBride, J. W. (2009) Nuclear Translocated Ehrlichia chaffeensis Ankyrin Protein Interacts with the Mid A-Stretch of Host Promoter and Intronic Alu Elements. Infect Immun 77: 4243-4255.

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

TABLE 1
Biological characteristics of Neorickettsia species

| | | | In vivo-infected | | |
|---|---|---|---|---|---|
| | Vertebrate | Invertebrate | Mammalian | Diseases & | Geographical |
| Species | Host | Vector/Host¹ | Cells | Symptoms | Distribution |
| N. helminthoeca | Canidae | Digenetic | Monocytes and | Salmon | California, |
| | | trematode | Macrophages | Poisoning | Washing-ton, |
| | | Nanophyetus | | Disease (pyrexia, | Oregon, |
| | | salmincola in | | anorexia, ocular | Idaho, |
| | | snails | | discharge, weight | Canada, |
| | | (Oxytrema | | loss, lethargy, | Brazil |
| | | silicula) and | | and dehydration, | |
| | | fish (salmonid) | | >90% mortality) | |
| N. risticii | Horse, Bat | Digenetic | Monocytes, | Potomac horse | USA, |
| | | trematode | Macrophages, | fever (fever, | Canada, |
| | | Acanthatrium | intestinal | depression, | Brazil, |
| | | oregonense in | epithelial cells, | anorexia, | Uruguay |
| | | snails (Elimia | and mast cells | dehydration, | |
| | | virginica) and | | watery diarrhea, | |
| | | aquatic insects | | laminitis, and/or | |
| | | (caddisflies, | | abortion, ~9% | |
| | | mayflies) | | fatality) | |
| N. sennetsu | Human | Unknown | Monocytes and | Sennetsu | Japan, |
| | | trematodes in | Macrophages | neorickettsiosis | Southeast |
| | | snails and grey | | (fever, fatigue, | Asia |
| | | mullet fish | | general malaise, | |
| | | | | and lymphadenopathy) | |

¹Transmission mode: all Neorickettsia spp. are transstadially and vertically transmitted through generations of trematodes.

TABLE 2
Genome properties of Neorickettsia spp.

| Strains¹ | NHO | NRI | NSE |
|---|---|---|---|
| RefSeq | NZ_CP007481.1 | NC_013009.1 | NC_007798.1 |
| Size (bp) | 884,232 | 879,977 | 859,006 |
| GC (%) | 41.7 | 41.3 | 41.1 |
| Protein | 774 | 760 | 754 |
| tRNA | 33 | 33 | 33 |
| rRNA | 3 | 3 | 3 |
| Other RNA | 1 | 2 | 3 |
| Pseudogene | 16 | 11 | 2 |
| Total Gene | 827 | 808 | 795 |
| Average gene length | 865 | 842 | 803 |
| Percent Coding² | 87.1 | 87.0 | 89.3 |
| Assigned functions | 548 | 534 | 540 |
| Unknown functions | 226 (29.2%) | 226 (29.7%) | 214 (28.4%) |

¹Abbreviations: NHO, N. helminthoeca Oregon (data obtained from in this study); NRI, N. risticii Illinois (Lin et al., 2009); NSE, N. sennetsu Miyayama (Dunning Hotopp et al., 2006).
²Percent coding includes tRNA, rRNA, small RNA, and all protein-coding genes.

TABLE 3
Role category breakdown of protein coding genes in Neorickettsia species

| | | | | Con- | Unique in |
|---|---|---|---|---|---|
| Role Category¹ | NHO | NSE | NRI | served² | NHO³ |
| Amino acid biosynthesis | 12 | 9 | 9 | 9 | 3 |
| Biosynthesis of cofactor and | 62 | 63 | 64 | 60 | 1 |
| vitamin | | | | | |
| Cell envelope | 45 | 31 | 31 | 28 | 17¹ |
| Cellular processes | 43 | 36 | 36 | 37 | 3 |
| Central intermediary | 8 | 5 | 5 | 5 | 3 |
| metabolism | | | | | |
| DNA metabolism | 33 | 36 | 37 | 33 | |
| Energy metabolism | 76 | 74 | 76 | 74 | |
| Fatty acid and phospholipid | 22 | 22 | 22 | 22 | |
| metabolism | | | | | |
| Mobile elements | 4 | 4 | 4 | 4 | |
| Protein fate | 87 | 86 | 88 | 86 | |
| Protein synthesis | 107 | 104 | 105 | 102 | 2 |
| Nucleotide biosynthesis | 37 | 36 | 36 | 36 | |
| Regulatory functions | 10 | 10 | 10 | 10 | |
| Signal transduction | 5 | 5 | 5 | 5 | |
| Transcription | 23 | 23 | 24 | 22 | |
| Transport and binding | 50 | 45 | 45 | 44 | 5 |
| proteins | | | | | |
| Unknown functions | 226 | 226 | 214 | 160 | 55 |
| Total Proteins⁴ | 774 | 760 | 754 | 668 | 89 |
| Total Assigned Functions: | 548 | 534 | 540 | 525 | |

¹Abbreviations: NHO, N. helminthoeca Oregon; NRI, N. risticii Illinois; NSE, N. sennetsu Miyayama.
²Proteins conserved among three Neorickettsia spp. and specific to N. helminthoeca are based on 3-way comparison analysis by BlastP (E < e⁻¹⁰).
³N. helminthoeca encodes nearly complete pathways for peptidoglycan biosynthesis.
⁴Certain proteins are assigned to multiple role categories.

TABLE 4
Putative outer membrane proteins of Neorickettsia helminthoeca¹

| | | | |
|---|---|---|---|
| | Gene | | MW |
| Locus ID | Symbol | Protein Name | (kDa) |

Molecular Characterization:

| | | | |
|---|---|---|---|
| NHE_RS00965 | p51 | P51 gram-negative porin family | 51.6 |
| | | protein | |
| NHE_RS03715 | nsp1 | Neorickettsia surface protein 1 | 27.6 |
| NHE_RS03720 | nsp2 | Neorickettsia surface protein 2 | 33.7 |
| NHE_RS03725 | nsp3 | Neorickettsia surface protein 3 | 24.7 |
| NHE_RS03855 | ssa | Strain-specific surface antigen | 35.5 |

pSort-B Prediction:

| | | | |
|---|---|---|---|
| NHE_RS00040 | | conserved hypothetical protein | 46.3 |
| NHE_RS01885 | | conserved hypothetical protein | 73.4 |
| NHE_RS03040 | yaeT | outer membrane protein assembly | 82.9 |
| | | complex, YaeT protein | |
| NHE_RS03940 | bamD | BamD lipoprotein | 26.5 |

¹Location of outer member proteins is predicted by the pSort-B algorithm (http://psort.org/psortb). Other putative OMPs (P51, NSP1/2/3, and SSA) are determined by homology searches to N. risticii and N. sennetsu protein database using BLASTP.

TABLE 5
Ortholog clusters conserved among Neorickettsia helminthoeca, N. risticii, and N. sennetsu
based on three-way comparison analysis¹

| | | |
|---|---|---|
| Ortholog Clusters | Protein Name | Role Category |

Amino acid biosynthesis

| | | |
|---|---|---|
| NSE_RS00705, NRI_RS00745, NHE_RS00695 | putative 3- | Amino acid biosynthesis\| |
| | phosphoshikimate 1- | Aromatic amino acid family |
| | carboxyvinyltransferase | |
| NSE_RS03830, NRI_RS03910, NHE_RS04025 | AraM domain protein | Amino acid biosynthesis\| |
| | | Aromatic amino acid family |
| NSE_RS01045, NRI_RS01085, NHE_RS01045 | aspartate-semialdehyde | Amino acid biosynthesis\| |
| | dehydrogenase | Aspartate family |
| NSE_RS03085, NRI_RS03170, NHE_RS03235 | aspartate aminotransferase | Amino acid biosynthesis\| |
| | | Aspartate family |
| NSE_RS03785, NRI_RS03870, NHE_RS03980 | dihydrodipicolinate | Amino acid biosynthesis\| |
| | synthase | Aspartate family |
| NSE_RS01455, NRI_RS01505, NHE_RS01490 | glutamine synthetase, type I | Amino acid biosynthesis\| |
| | | Glutamate family |
| NSE_RS02825, NRI_RS02915, NHE_RS02945 | glutamine synthetase | Amino acid biosynthesis\| |
| | domain protein | Glutamate family |
| NSE_RS02670, NRI_RS02760, NHE_RS02780 | bifunctional glutamate | Amino acid biosynthesis\| |
| | synthase subunit beta/2- | Glutamate family |
| | polyprenylphenol | |
| | hydroxylase (GS/PH) | |
| NSE_RS00865, NRI_RS00905, NHE_RS00860 | serine | Amino acid biosynthesis\|Serine |
| | hydroxymethyltransferase | family |

Biosynthesis of cofactors and prosthetic groups

| | | |
|---|---|---|
| NSE_RS02480, NRI_RS02540, NHE_RS02585 | biotin synthase | Biosynthesis of cofactors, |
| | | prosthetic groups, and carriers\| |
| | | Biotin |
| NSE_RS02485, NRI_RS02545, NHE_RS02590, | 8-amino-7-oxononanoate | Biosynthesis of cofactors, |
| NHE_RS03545, NRI_RS03445 | synthase | prosthetic groups, and carriers\| |
| | | Biotin |
| NSE_RS02495, NRI_RS02555, NHE_RS02600 | biotin biosynthesis protein | Biosynthesis of cofactors, |
| | BioC | prosthetic groups, and carriers\| |
| | | Biotin |
| NSE_RS02505, NRI_RS02565, NHE_RS02610, | adenosylmethionine-8- | Biosynthesis of cofactors, |
| NHE_RS03625, NRI_RS03545 | amino-7-oxononanoate | prosthetic groups, and carriers\| |
| | aminotransferase | Biotin |
| NSE_RS03365, NRI_RS03445, NHE_RS03545, | 5-aminolevulinic acid | Biosynthesis of cofactors, |
| NRI_RS02545, NHE_RS02590 | synthase | prosthetic groups, and carriers\| |
| | | Biotin |
| NSE_RS03465, NRI_RS03545, NHE_RS03625, | acetylornithine | Biosynthesis of cofactors, |
| NRI_RS02565, NHE_RS02610 | aminotransferase | prosthetic groups, and carriers\| |
| | | Biotin |
| NSE_RS01965, NRI_RS02005, NHE_RS02010 | dihydropteroate synthase | Biosynthesis of cofactors, |
| | | prosthetic groups, and carriers\| |
| | | Folic acid |
| NSE_RS02030, NRI_RS02075, NHE_RS02085 | putative dihydroneopterin | Biosynthesis of cofactors, |
| | aldolase | prosthetic groups, and carriers\| |
| | | Folic acid |
| NSE_RS02405, NRI_RS02460, NHE_RS02505 | GTP cyclohydrolase I | Biosynthesis of cofactors, |
| | | prosthetic groups, and carriers\| |
| | | Folic acid |
| NSE_RS02905, NRI_RS02995, NHE_RS03030 | folylpolyglutamate | Biosynthesis of cofactors, |
| | synthase | prosthetic groups, and carriers\| |
| | | Folic acid |
| NSE_RS03340, NRI_RS03420, NHE_RS03505 | FolD bifunctional protein | Biosynthesis of cofactors, |
| | | prosthetic groups, and carriers\| |
| | | Folic acid |
| NSE_RS00730, NRI_RS00765, NHE_RS00720 | glutathione synthetase | Biosynthesis of cofactors, |
| | | prosthetic groups, and carriers\| |
| | | Glutathione and analogs |
| NSE_RS01275, NRI_RS01320, NHE_RS01280 | glutamate--cysteine ligase | Biosynthesis of cofactors, |
| | | prosthetic groups, and carriers\| |
| | | Glutathione and analogs |
| NSE_RS01560, NRI_RS01610, NHE_RS01610 | putative porphobilinogen | Biosynthesis of cofactors, |
| | deaminase | prosthetic groups, and carriers\| |
| | | Heme, porphyrin, and cobalamin |
| NSE_RS01595, NRI_RS01645, NHE_RS01645 | porphobilinogen synthase | Biosynthesis of cofactors, |
| | | prosthetic groups, and carriers\| |
| | | Heme, porphyrin, and cobalamin |
| NSE_RS01830, NRI_RS01870, NHE_RS01875 | coproporphyrinogen III | Biosynthesis of cofactors, |
| | oxidase, aerobic | prosthetic groups, and carriers\| |
| | | Heme, porphyrin, and cobalamin |
| NSE_RS02530, NRI_RS02590, NHE_RS02635 | protoheme IX | Biosynthesis of cofactors, |
| | farnesyltransferase | prosthetic groups, and carriers\| |
| | | Heme, porphyrin, and cobalamin |
| NSE_RS03200, NRI_RS03285, NHE_RS03360 | ferrochelatase | Biosynthesis of cofactors, |
| | | prosthetic groups, and carriers\| |
| | | Heme, porphyrin, and cobalamin |
| NSE_RS03950, NRI_RS04030, NHE_RS00005 | uroporphyrinogen | Biosynthesis of cofactors, |
| | decarboxylase | prosthetic groups, and carriers\| |
| | | Heme, porphyrin, and cobalamin |
| NSE_RS01340, NRI_RS01390, NHE_RS01350 | lipoic acid synthetase | Biosynthesis of cofactors, |
| | | prosthetic groups, and carriers\| |
| | | Lipoate |
| NSE_RS01405, NRI_RS01455, NHE_RS01435 | Coq7 family protein | Biosynthesis of cofactors, |
| | | prosthetic groups, and carriers\| |
| | | Menaquinone and ubiquinone |
| NSE_RS01555, NRI_RS01605, NHE_RS01605 | ubiquinone biosynthesis | Biosynthesis of cofactors, |
| | hydroxylase, | prosthetic groups, and carriers\| |
| | UbiH/LibiF/VisC/COQ6 | Menaquinone and ubiquinone |
| | family | |
| NSE_RS02555, NRI_RS02615, NHE_RS02665 | 3-demethylubiquinone-9 3- | Biosynthesis of cofactors, |
| | methyltransferase | prosthetic groups, and carriers\| |
| | | Menaquinone and ubiquinone |
| NSE_RS02585, NRI_RS02655, NHE_RS02705 | putative ubiquinone | Biosynthesis of cofactors, |
| | biosynthesis protein | prosthetic groups, and carriers\| |
| | | Menaquinone and ubiquinone |
| NSE_RS03275, NRI_RS03350, NHE_RS03435 | 4-hydroxybenzoate | Biosynthesis of cofactors, |
| | octaprenyltransferase | prosthetic groups, and carriers\| |
| | | Menaquinone and ubiquinone |
| NSE_RS03150, NRI_RS03235, NHE_RS03305 | molybdopterin biosynthesis | Biosynthesis of cofactors, |
| | protein MoeB | prosthetic groups, and carriers\| |
| | | Molybdopterin |
| NSE_RS00525, NRI_RS00570, NHE_RS00520 | 2C-methyl-D-erythritol | Biosynthesis of cofactors, |
| | 2,4-cyclodiphosphate | prosthetic groups, and carriers\| |
| | synthase | Other |
| NSE_RS00695, NRI_RS00735, NHE_RS00685 | putative 2-C-methyl-D- | Biosynthesis of cofactors, |
| | erythritol 4-phosphate | prosthetic groups, and carriers\| |
| | cytidylyltransferase | Other |
| NSE_RS00950, NRI_RS00990, NHE_RS00950, | polyprenyl synthetase | Biosynthesis of cofactors, |
| NRI_RS02905, NHE_RS02935 | family protein | prosthetic groups, and carriers\| |
| | | Other |
| NSE_RS01240, NRI_RS01285, NHE_RS01245 | iron-sulfur cluster | Biosynthesis of cofactors, |
| | assembly accessory protein | prosthetic groups, and carriers\| |
| | | Other |
| NSE_RS01245, NRI_RS01290, NHE_RS01250 | FeS cluster assembly | Biosynthesis of cofactors, |
| | scaffold IscU | prosthetic groups, and carriers\| |
| | | Other |
| NSE_RS01250, NRI_RS01295, NHE_RS01255 | cysteine desulfurase | Biosynthesis of cofactors, |
| | | prosthetic groups, and carriers\| |
| | | Other |
| NSE_RS01255, NRI_RS01300, NHE_RS01260 | rrf2 family transcriptional | Biosynthesis of cofactors, |
| | regulator with | prosthetic groups, and carriers\| |
| | aminotransferase, class V | Other |
| | family protein | |
| NSE_RS01770, NRI_RS01810, NHE_RS01815 | 4-hydroxy-3-methylbut-2- | Biosynthesis of cofactors, |
| | enyl diphosphate reductase | prosthetic groups, and carriers\| |
| | | Other |
| NSE_RS01790, NRI_RS01830, NHE_RS01835 | 1-deoxy-D-xylulose 5- | Biosynthesis of cofactors, |
| | phosphate | prosthetic groups, and carriers\| |
| | reductoisomerase | Other |
| NSE_RS01845, NRI_RS01885, NHE_RS01890 | putative iron-sulfur cluster | Biosynthesis of cofactors, |
| | assembly accessory protein | prosthetic groups, and carriers\| |
| | | Other |
| NSE_RS02815, NRI_RS02905, NHE_RS02935, | putative | Biosynthesis of cofactors, |
| NRI_RS00990, NHE_RS00950 | geranyltranstransferase | prosthetic groups, and carriers\| |
| | | Other |
| NSE_RS02925, NRI_RS03015, NHE_RS03050 | putative 4- | Biosynthesis of cofactors, |
| | diphosphocytidyl-2C- | prosthetic groups, and carriers\| |
| | methyl-D-erythritol kinase | Other |
| NSE_RS03245, NRI_RS03325, NHE_RS03405 | 1-hydroxy-2-methyl-2-(E)- | Biosynthesis of cofactors, |
| | butenyl 4-diphosphate | prosthetic groups, and carriers\| |
| | synthase | Other |
| NSE_RS01280, NRI_RS01325, NHE_RS01285 | dephospho-CoA kinase | Biosynthesis of cofactors, |
| | | prosthetic groups, and carriers\| |
| | | Pantothenate and coenzyme A |
| NSE_RS03960, NRI_RS04040, NHE_RS00015 | pantetheine-phosphate | Biosynthesis of cofactors, |
| | adenylyltransferase | prosthetic groups, and carriers\| |
| | | Pantothenate and coenzyme A |
| NSE_RS00395, NRI_RS00400, NHE_RS00400 | NAD+ synthetase | Biosynthesis of cofactors, |
| | | prosthetic groups, and carriers\| |
| | | Pyridine nucleotides |
| NSE_RS00470, NRI_RS00515, NHE_RS00465 | nicotinate-nucleotide | Biosynthesis of cofactors, |
| | pyrophosphorylase | prosthetic groups, and carriers\| |
| | | Pyridine nucleotides |
| NSE_RS02890, NRI_RS02980, NHE_RS04215 | putative nicotinate | Biosynthesis of cofactors, |
| | (nicotinamide) nucleotide | prosthetic groups, and carriers\| |
| | adenylyltransferase | Pyridine nucleotides |
| NSE_RS01330, NRI_RS01380, NHE_RS01340 | pyridoxal phosphate | Biosynthesis of cofactors, |
| | biosynthetic protein PdxJ | prosthetic groups, and carriers\| |
| | | Pyridoxine |
| NSE_RS01515, NRI_RS01565, NHE_RS01560 | putative pyridoxamine 5- | Biosynthesis of cofactors, |
| | phosphate oxidase | prosthetic groups, and carriers\| |
| | | Pyridoxine |
| NSE_RS00170, NRI_RS00155, NHE_RS00165 | riboflavin biosynthesis | Biosynthesis of cofactors, |
| | protein RibF | prosthetic groups, and carriers\| |
| | | Riboflavin, FMN, and FAD |
| NSE_RS00365, NRI_RS00360, NHE_RS00375 | cytidine/deoxycytidylate | Biosynthesis of cofactors, |
| | deaminase family protein | prosthetic groups, and carriers\| |
| | | Riboflavin, FMN, and FAD |
| NSE_RS01630, NRI_RS01680, NHE_RS01680 | 6,7-dimethyl-8- | Biosynthesis of cofactors, |
| | ribityllumazine synthase | prosthetic groups, and carriers\| |
| | | Riboflavin, FMN, and FAD |
| NSE_RS02020, NRI_RS02065, NHE_RS02075 | riboflavin biosynthesis | Biosynthesis of cofactors, |
| | protein RibD | prosthetic groups, and carriers\| |
| | | Riboflavin, FMN, and FAD |
| NSE_RS02635, NRI_RS02705, NHE_RS02755 | 3,4-dihydroxy-2-butanone | Biosynthesis of cofactors, |
| | 4-phosphate synthase | prosthetic groups, and carriers\| |
| | | Riboflavin, FMN, and FAD |
| NSE_RS03025, NRI_RS03115, NHE_RS03170 | GTP cyclohydrolase II | Biosynthesis of cofactors, |
| | | prosthetic groups, and carriers\| |
| | | Riboflavin, FMN, and FAD |
| NSE_RS03480, NRI_RS03560, NHE_RS03640 | riboflavin synthase, alpha | Biosynthesis of cofactors, |
| | subunit | prosthetic groups, and carriers\| |
| | | Riboflavin, FMN, and FAD |
| NSE_RS00190, NRI_RS00175, NHE_RS00185 | thiamine biosynthesis | Biosynthesis of cofactors, |
| | protein ThiS | prosthetic groups, and carriers\| |
| | | Thiamine |
| NSE_RS00875, NRI_RS00915, NHE_RS00870 | putative thiamine- | Biosynthesis of cofactors, |
| | phosphate | prosthetic groups, and carriers\| |
| | pyrophosphorylase | Thiamine |
| NSE_RS01955, NRI_RS02000, NHE_RS02005 | thiamin biosynthesis ThiG | Biosynthesis of cofactors, |
| | | prosthetic groups, and carriers\| |
| | | Thiamine |
| NSE_RS01995, NRI_RS02035, NHE_RS02040 | coenzyme PQQ synthesis | Biosynthesis of cofactors, |
| | protein C | prosthetic groups, and carriers\| |
| | | Thiamine |
| NSE_RS03880, NRI_RS03960, NHE_RS04080 | putative | Biosynthesis of cofactors, |
| | phosphomethylpyrimidine | prosthetic groups, and carriers\| |
| | kinase | Thiamine |

Cell envelope

| | | |
|---|---|---|
| NSE_RS01935, NRI_RS01980, NHE_RS01985 | UDP-N-acetylmuramoyl- | Cell envelope\|Biosynthesis and |
| | tripeptide--D-alanyl-D- | degradation of murein sacculus |
| | alanine ligase truncation, | and peptidoglycan |
| | partial | |
| NSE_RS02415, NRI_RS02470, NHE_RS02520 | putative UDP-N- | Cell envelope\|Biosynthesis and |
| | acetylenolpyruvoylglucosamine | degradation of murein sacculus |
| | reductase | and peptidoglycan |
| NSE_RS03755, NRI_RS03840, NHE_RS03930 | S-adenosyl- | Cell envelope\|Biosynthesis and |
| | methyltransferase MraW | degradation of murein sacculus |
| | | and peptidoglycan |
| NSE_RS00820, NRI_RS00860, NHE_RS00815 | exopolysaccharide | Cell envelope\|Biosynthesis and |
| | synthesis protein | degradation of surface |
| | | polysaccharides and |
| | | lipopolysaccharides |
| NSE_RS03845, NRI_RS03925, NHE_RS04040 | undecaprenyl diphosphate | Cell envelope\|Biosynthesis and |
| | synthase | degradation of surface |
| | | polysaccharides and |
| | | lipopolysaccharides |
| NSE_RS00220, NRI_RS00205, NHE_RS00215 | putative membrane protein | Cell envelope\|Other |
| NSE_RS00295, NRI_RS00285, NHE_RS00295 | putative membrane protein | Cell envelope\|Other |
| NSE_RS00300, NRI_RS00290, NHE_RS00305 | putative lipoprotein | Cell envelope\|Other |
| NSE_RS00465, NRI_RS00510, NHE_RS00460 | putative membrane protein | Cell envelope\|Other |
| NSE_RS00815, NRI_RS00855, NHE_RS00810 | hypothetical protein | Cell envelope\|Other |
| NSE_RS00965, NRI_RS01005, NHE_RS00965 | 51 kDa major antigen | Cell envelope\|Other |
| | (P51) | |
| NSE_RS01635, NRI_RS01685, NHE_RS01685 | inner membrane protein, | Cell envelope\|Other |
| | 60 kDa | |
| NSE_RS02220, NRI_RS02265, NHE_RS02300 | putative membrane protein | Cell envelope\|Other |
| NSE_RS02265, NRI_RS02310, NHE_RS02345 | major surface protein | Cell envelope\|Other |
| NSE_RS02305, NRI_RS02355, NHE_RS02395 | membrane protein, MviN | Cell envelope\|Other |
| | family | |
| NSE_RS02355, NRI_RS02405, NHE_RS02445 | putative membrane protein | Cell envelope\|Other |
| NSE_RS02995, NRI_RS03085, NHE_RS03135 | putative membrane protein | Cell envelope\|Other |
| NSE_RS03220, NRI_RS03305, NHE_RS03385 | membrane protein, TerC | Cell envelope\|Other |
| | family | |
| NSE_RS03550, NRI_RS03630, NHE_RS03715 | Neorickettsia surface | Cell envelope\|Other |
| | protein 1 | |
| NSE_RS03555, NRI_RS03635, NHE_RS03720 | Neorickettsia surface | Cell envelope\|Other |
| | protein 2 | |
| NSE_RS03560, NRI_RS03640, NHE_RS03725 | Neorickettsia surface | Cell envelope\|Other |
| | protein 3 | |
| NSE_RS03620, NRI_RS03705, NHE_RS03785 | putative peptidoglycan- | Cell envelope\|Other |
| | associated lipoprotein | |
| NSE_RS03690, NRI_RS03700, NRI_RS03775, | strain-specific surface | Cell envelope\|Other |
| NRI_RS03780, | antigen | |
| NRI_RS03785, NHE_RS03855 | | |
| NSE_RS03775, NRI_RS03860, NHE_RS03965, | putative membrane protein | Cell envelope\|Other |
| NRI_RS03865, NHE_RS03970 | | |
| NSE_RS03780, NRI_RS03865, NHE_RS03970, | putative membrane protein | Cell envelope\|Other |
| NRI_RS03860, NHE_RS03965 | | |
| NSE_RS03810, NRI_RS03890, NHE_RS04005 | putative vacJ lipoprotein | Cell envelope\|Other |

Cellular processes

| | | |
|---|---|---|
| NSE_RS00245, NRI_RS00230, NHE_RS00240 | putative osmotically | Cellular processes\|Adaptations to |
| | inducible protein | atypical conditions |
| NSE_RS01530, NRI_RS01580, NHE_RS01580 | acid phosphatase SurE | Cellular processes\|Adaptations to |
| | | atypical conditions |
| NSE_RS00015, NRI_RS00005, NHE_RS00025 | chromosome partitioning | Cellular processes\|Cell division |
| | protein, ParB family | |
| NSE_RS00460, NRI_RS00505, NHE_RS00455 | ribonuclease, Rne/Rng | Cellular processes\|Cell division |
| | family | |
| NSE_RS01420, NRI_RS01470, NHE_RS01450 | cell division protein FtsZ | Cellular processes\|Cell division |
| NSE_RS01730, NRI_RS01770, NHE_RS01770 | cell division protein FtsA | Cellular processes\|Cell division |
| NSE_RS02400, NRI_RS02455, NHE_RS02500 | putative cell division | Cellular processes\|Cell division |
| | protein | |
| NSE_RS03905, NRI_RS03985, NHE_RS04110 | GTP-binding protein Era | Cellular processes\|Cell division |
| NSE_RS03990, NRI_RS04075, NHE_RS04210 | putative cell division | Cellular processes\|Cell division |
| | protein FtsK | |
| NSE_RS02295, NRI_RS02340, NHE_RS02380 | antioxidant, AhpC/Tsa | Cellular processes\|Detoxification |
| | family | |
| NSE_RS03430, NRI_RS03510, NHE_RS03595 | superoxide dismutase, Fe | Cellular processes\|Detoxification |
| NSE_RS01690, NRI_RS01740, NHE_RS04195, | putative competence | Cellular processes\|DNA |
| NHE_RS04190 | protein F | transformation |
| NSE_RS03765, NRI_RS03850, NHE_RS03940 | putative competence | Cellular processes\|DNA |
| | protein ComL | transformation |
| NSE_RS01610, NRI_RS01660, NHE_RS01660 | ATP synthase F0, C chain | Cellular processes\|Pathogenesis |
| NSE_RS03105, NRI_RS03190, NHE_RS03255 | ATP synthase F1, epsilon | Cellular processes\|Pathogenesis |
| | subunit | |
| NSE_RS00290, NRI_RS00280, NHE_RS00290 | drug resistance transporter, | Cellular processes\|Toxin |
| | Bcr/CflA family | production and resistance |
| NSE_RS00750, NRI_RS00790, NHE_RS00745 | transporter, | Cellular processes\|Toxin |
| | AcrB/AcrD/AcrF family | production and resistance |
| NSE_RS00520, NRI_RS00565, NHE_RS00515 | 5,10- | Central intermediary metabolism\| |
| | methenyltetrahydrofolate | One-carbon metabolism |
| | synthetase | |
| NSE_RS01800, NRI_RS01840, NHE_RS01845, | oxidoreductase, short-chain | Central intermediary\|metabolism |
| NRI_RS02775, NHE_RS02800 | dehydrogenase/reductase | Other |
| | family | |
| NSE_RS01975, NRI_RS02015, NHE_RS02020 | S-adenosylmethionine | Central intermediary metabolism\| |
| | synthetase | Other |
| NSE_RS02685, NRI_RS02775, NHE_RS02800, | 3-oxoacyl-[acyl-carrier | Central intermediary metabolism\| |
| NRI_RS01840 | protein] reductase | Other |
| NSE_RS02980, NRI_RS03070, NHE_RS03110 | inorganic pyrophosphatase | Central intermediary metabolism\| |
| | | Phosphorus compounds |

DNA metabolism

| | | |
|---|---|---|
| NSE_RS02595, NRI_RS02665, NHE_RS02715 | DNA-binding protein HU | DNA metabolism\|Chromosome- |
| | | associated proteins |
| NSE_RS00560, NRI_RS00600, NHE_RS00545, | tyrosine recombinase XerD | DNA metabolism\|DNA |
| NHE_RS01850, NRI_RS01845 | | replication, recombination, and |
| | | repair |
| NSE_RS00620, NRI_RS00660, NHE_RS00605 | DnaK suppressor protein | DNA metabolism\|DNA |
| | | replication, recombination, and |
| | | repair |
| NSE_RS00645, NRI_RS00685, NHE_RS00630 | DNA polymerase III, alpha | DNA metabolism\|DNA |
| | subunit | replication, recombination, and |
| | | repair |
| NSE_RS00660, NRI_RS00700, NHE_RS00645 | DNA polymerase III, beta | DNA metabolism\|DNA |
| | subunit | replication, recombination, and |
| | | repair |
| NSE_RS00780, NRI_RS00820, NHE_RS00775 | putative DNA replication | DNA metabolism\|DNA |
| | and repair protein RecF | replication, recombination, and |
| | | repair |
| NSE_RS00810, NRI_RS00850, NHE_RS00805 | primosomal protein N′ | DNA metabolism\|DNA |
| | | replication, recombination, and |
| | | repair |
| NSE_RS00915, NRI_RS04065, NHE_RS00910, | DNA repair protein RadC | DNA metabolism\|DNA |
| NRI_RS04060 | | replication, recombination, and |
| | | repair |
| NSE_RS00975, NRI_RS01015, NHE_RS00975 | endonuclease III | DNA metabolism\|DNA |
| | | replication, recombination, and |
| | | repair |
| NSE_RS01040, NRI_RS01080, NHE_RS01040 | chromosomal replication | DNA metabolism\|DNA |
| | initiator protein DnaA | replication, recombination, and |
| | | repair |
| NSE_RS01685, NRI_RS01735, NHE_RS01735 | exodeoxyribonuclease III | DNA metabolism\|DNA |
| | | replication, recombination, and |
| | | repair |
| NSE_RS01805, NRI_RS01845, NHE_RS01850, | site-specific recombinase, | DNA metabolism\|DNA |
| NRI_RS00600, NHE_RS00545 | phage integrase family | replication, recombination, and |
| | | repair |
| NSE_RS01855, NRI_RS01895, NHE_RS01900 | putative DNA repair | DNA metabolism\|DNA |
| | protein RecO | replication, recombination, and |
| | | repair |
| NSE_RS01885, NRI_RS01930, NHE_RS01930 | ATP-dependent DNA | DNA metabolism\|DNA |
| | helicase, UvrD/REP family | replication, recombination, and |
| | | repair |
| NSE_RS01895, NRI_RS01940, NHE_RS01945 | DNA polymerase III, | DNA metabolism\|DNA |
| | epsilon subunit | replication, recombination, and |
| | | repair |
| NSE_RS01990, NRI_RS02030, NHE_RS02035 | putative DNA polymerase | DNA metabolism\|DNA |
| | III, gamma/tau subunit | replication, recombination, and |
| | | repair |
| NSE_RS02025, NRI_RS02070, NHE_RS02080 | DNA ligase, NAD- | DNA metabolism\|DNA |
| | dependent | replication, recombination, and |
| | | repair |
| NSE_RS02170, NRI_RS02215, NHE_RS02250 | recA protein | DNA metabolism\|DNA |
| | | replication, recombination, and |
| | | repair |
| NSE_RS02360, NRI_RS02410, NHE_RS02455 | holliday junction DNA | DNA metabolism\|DNA |
| | helicase RuvA | replication, recombination, and |
| | | repair |
| NSE_RS02365, NRI_RS02415, NHE_RS02460 | holliday junction DNA | DNA metabolism\|DNA |
| | helicase RuvB | replication, recombination, and |
| | | repair |
| NSE_RS02430, NRI_RS02485, NHE_RS02535 | DNA topoisomerase I | DNA metabolism\|DNA |
| | | replication, recombination, and |
| | | repair |
| NSE_RS02625, NRI_RS02695, NHE_RS02745 | polyA polymerase family | DNA metabolism\|DNA |
| | protein | replication, recombination, and |
| | | repair |
| NSE_RS02720, NRI_RS02810, NHE_RS02840 | DNA polymerase I | DNA metabolism\|DNA |
| | | replication, recombination, and |
| | | repair |
| NSE_RS02795, NRI_RS02885, NHE_RS02915 | ATP-dependent DNA | DNA metabolism\|DNA |
| | helicase RecG | replication, recombination, and |
| | | repair |
| NSE_RS02895, NRI_RS02985, NHE_RS03020 | single-stranded-DNA- | DNA metabolism\|DNA |
| | specific exonuclease RecJ | replication, recombination, and |
| | | repair |
| NSE_RS02930, NRI_RS03020, NHE_RS03055 | DNA gyrase, B subunit | DNA metabolism\|DNA |
| | | replication, recombination, and |
| | | repair |
| NSE_RS03090, NRI_RS03175, NHE_RS03240 | single-strand binding | DNA metabolism\|DNA |
| | protein | replication, recombination, and |
| | | repair |
| NSE_RS03670, NRI_RS03755, NHE_RS03835 | recombination protein | DNA metabolism\|DNA |
| | RecR | replication, recombination, and |
| | | repair |
| NSE_RS03805, NRI_RS03885, NHE_RS04000 | uracil-DNA glycosylase, | DNA metabolism\|DNA |
| | family 4 | replication, recombination, and |
| | | repair |
| NSE_RS03885, NRI_RS03965, NHE_RS04085 | crossover junction | DNA metabolism\|DNA |
| | endodeoxyribonuclease | replication, recombination, and |
| | RuvC | repair |
| NSE_RS03900, NRI_RS03980, NHE_RS04105 | DNA gyrase, A subunit | DNA metabolism\|DNA |
| | | replication, recombination, and |
| | | repair |
| NSE_RS03915, NRI_RS03995, NHE_RS04120 | DNA primase | DNA metabolism\|DNA |
| | | replication, recombination, and |
| | | repair |

Energy metabolism

| | | |
|---|---|---|
| NSE_RS00910, NRI_RS00950, NHE_RS00905 | glycerol-3-phosphate | Energy metabolism\|Aerobic |
| | dehydrogenase (NAD(P)+) | |
| NSE_RS03315, NRI_RS03395, NHE_RS03480 | propionyl-CoA | Energy metabolism\|Amino acids |
| | carboxylase, alpha subunit | and amines |
| NSE_RS00510, NRI_RS00555, NHE_RS00505, | ATP synthase F1, alpha | Energy metabolism\|ATP-proton |
| NRI_RS03195, NHE_RS03260 | subunit | motive force interconversion |
| NSE_RS00515, NRI_RS00560, NHE_RS00510 | ATP synthase F1, delta | Energy metabolism\|ATP-proton |
| | subunit | motive force interconversion |
| NSE_RS01605, NRI_RS01655, NHE_RS01655 | ATP synthase F0, A | Energy metabolism\|ATP-proton |
| | subunit | motive force interconversion |
| NSE_RS01620, NRI_RS01670, NHE_RS01670 | putative ATPase F0, B | Energy metabolism\|ATP-proton |
| | chain | motive force interconversion |
| NSE_RS02410, NRI_RS02465, NHE_RS02510 | ATP synthase F1, gamma | Energy metabolism\|ATP-proton |
| | subunit | motive force interconversion |
| NSE_RS03110, NRI_RS03195, NHE_RS03260, | ATP synthase F1, beta | Energy metabolism\|ATP-proton |
| NRI_RS00555, NHE_RS00505 | subunit | motive force interconversion |
| NSE_RS00060, NRI_RS00050, NHE_RS00065 | NADH dehydrogenase I, J | Energy metabolism\|Electron |
| | subunit | transport |
| NSE_RS00065, NRI_RS00055, NHE_RS00070 | NADH dehydrogenase I, K | Energy metabolism\|Electron |
| | subunit | transport |
| NSE_RS00070, NRI_RS00060, NHE_RS00075, | NADH dehydrogenase I, L | Energy metabolism\|Electron |
| NHE_RS02400, NRI_RS02360, | subunit | transport |
| NRI_RS02890, NHE_RS02920, | | |
| NRI_RS02955, NHE_RS02990, | | |
| NRI_RS02895, NHE_RS02925 | | |
| NSE_RS00235, NRI_RS00220, NHE_RS00230 | NADH dehydrogenase I, G | Energy metabolism\|Electron |
| | subunit | transport |
| NSE_RS00240, NRI_RS00225, NHE_RS00235 | NADH dehydrogenase I, H | Energy metabolism\|Electron |
| | subunit | transport |
| NSE_RS00905, NRI_RS00945, NHE_RS00900 | thioredoxin | Energy metabolism\|Electron |
| | | transport |
| NSE_RS01035, NRI_RS01075, NHE_RS01035 | cytochrome c oxidase | Energy metabolism\|Electron |
| | assembly protein CtaG | transport |
| NSE_RS01225, NRI_RS01270, NHE_RS01230 | iron-sulfur cluster binding | Energy metabolism\|Electron |
| | protein | transport |
| NSE_RS01290, NRI_RS01335, NHE_RS01295 | glutaredoxin 3 | Energy metabolism\|Electron |
| | | transport |
| NSE_RS01370, NRI_RS01420, NHE_RS01380 | ferredoxin | Energy metabolism\|Electron |
| | | transport |
| NSE_RS01545, NRI_RS01595, NHE_RS01595 | quinone oxidoreductase | Energy metabolism\|Electron |
| | | transport |
| NSE_RS01550, NRI_RS01600, NHE_RS01600 | putative oxidoreductase | Energy metabolism\|Electron |
| | | transport |
| NSE_RS01740, NRI_RS01780, NHE_RS01780 | NADH dehydrogenase I, A | Energy metabolism\|Electron |
| | subunit | transport |
| NSE_RS01745, NRI_RS01785, NHE_RS01785 | NADH dehydrogenase I, B | Energy metabolism\|Electron |
| | subunit | transport |
| NSE_RS01750, NRI_RS01790, NHE_RS01790 | NADH dehydrogenase I, C | Energy metabolism\|Electron |
| | subunit | transport |
| NSE_RS01910, NRI_RS01955, NHE_RS01960 | cytochrome c | Energy metabolism\|Electron |
| | | transport |
| NSE_RS02290, NRI_RS02335, NHE_RS02375, | thioredoxin-disulfide | Energy metabolism\|Electron |
| NHE_RS03310, NRI_RS03240 | reductase | transport |
| NSE_RS02325, NRI_RS02375, NHE_RS02415 | NADH dehydrogenase I, D | Energy metabolism\|Electron |
| | subunit | transport |
| NSE_RS02350, NRI_RS02400, NHE_RS02440 | cytochrome c-type | Energy metabolism\|Electron |
| | biogenesis protein, | transport |
| | CcmF/CycK/CcsA family | |
| NSE_RS02520, NRI_RS02580, NHE_RS02625 | cytochrome c oxidase, | Energy metabolism\|Electron |
| | subunit II | transport |
| NSE_RS02525, NRI_RS02585, NHE_RS02630 | cytochrome c oxidase, | Energy metabolism\|Electron |
| | subunit I | transport |
| NSE_RS02535, NRI_RS02595, NHE_RS02640 | ubiquinol-cytochrome c | Energy metabolism\|Electron |
| | reductase, iron-sulfur | transport |
| | subunit | |
| NSE_RS02540, NRI_RS02600, NHE_RS02645 | ubiquinol-cytochrome c | Energy metabolism\|Electron |
| | reductase, cytochrome b | transport |
| NSE_RS02545, NRI_RS02605, NHE_RS02650 | ubiquinol-cytochrome c | Energy metabolism\|Electron |
| | reductase, cytochrome c1 | transport |
| NSE_RS02580, NRI_RS02650, NHE_RS02700 | NADH dehydrogenase I, E | Energy metabolism\|Electron |
| | subunit | transport |
| NSE_RS02725, NRI_RS02815, NHE_RS02845 | cytochrome c oxidase, | Energy metabolism\|Electron |
| | subunit III | transport |
| NSE_RS02310, NRI_RS02360, NHE_RS02400, | NADH- | Energy metabolism\|Electron |
| NHE_RS02990, NHE_RS00075, | ubiquinone/plastoquinone | transport |
| NRI_RS00060, NRI_RS02955, | oxidoreductase family | |
| NHE_RS02920, NRI_RS02890 | protein | |
| NSE_RS02800, NRI_RS02890, NHE_RS02920, | NADH dehydrogenase I, | Energy metabolism\|Electron |
| NHE_RS00075, NHE_RS02400, | M subunit | transport |
| NRI_RS02955, NRI_RS00060, | | |
| NRI_RS02360, NHE_RS02990, | | |
| NHE_RS02925 | | |
| NSE_RS02805, NRI_RS02895, NHE_RS02925, | NADH dehydrogenase I, N | Energy metabolism\|Electron |
| NHE_RS02990, NRI_RS02955, | subunit | transport |
| NRI_RS00060, | | |
| NHE_RS00075, NHE_RS02920 | | |
| NSE_RS02865, NRI_RS02955, NHE_RS02990, | NADH- | Energy metabolism\|Electron |
| NRI_RS02360, NRI_RS02890, | ubiquinone/plastoquinone | transport |
| NHE_RS02920, | oxidoreductase family | |
| NHE_RS02400, NHE_RS00075, NHE_RS02925, | protein | |
| NRI_RS00060, NRI_RS02895 | | |
| NSE_RS02900, NRI_RS02990, NHE_RS03025 | NADH dehydrogenase I, F | Energy metabolism\|Electron |
| | subunit | transport |
| NSE_RS03155, NRI_RS03240, NHE_RS03310, | pyridine nucleotide- | Energy metabolism\|Electron |
| NHE_RS02375, NRI_RS02335 | disulphide oxidoreductase | transport |
| | family protein | |
| NSE_RS03335, NRI_RS03415, NHE_RS03500 | NADH dehydrogenase I, I | Energy metabolism\|Electron |
| | subunit | transport |
| NSE_RS03375, NRI_RS03455, NHE_RS03555 | putative cytochrome c-type | Energy metabolism\|Electron |
| | biogenesis protein CcmE | transport |
| NSE_RS03490, NRI_RS03570, NHE_RS03650 | putative cytochrome | Energy metabolism\|Electron |
| | oxidase assembly protein | transport |
| NSE_RS03645, NRI_RS03730, NHE_RS03810 | thioredoxin 1 | Energy metabolism\|Electron |
| | | transport |
| NSE_RS03790, NRI_RS03875, NHE_RS03985 | cytochrome b561 family | Energy metabolism\|Electron |
| | protein | transport |
| NSE_RS00555, NRI_RS00595, NHE_RS00550 | putative fructose- | Energy metabolism\| |
| | bisphosphate aldolase, | Glycolysis/gluconeogenesis |
| | class I | |
| NSE_RS01015, NRI_RS01055, NHE_RS01015 | triosephosphate isomerase | Energy metabolism\| |
| | | Glycolysis/gluconeogenesis |
| NSE_RS01760, NRI_RS01800, NHE_RS01800 | glyceraldehyde-3- | Energy metabolism\| |
| | phosphate dehydrogenase, | Glycolysis/gluconeogenesis |
| | type I | |
| NSE_RS01765, NRI_RS01805, NHE_RS01805 | phosphoglycerate kinase | Energy metabolism\| |
| | | Glycolysis/gluconeogenesis |
| NSE_RS02975, NRI_RS03065, NHE_RS03105 | enolase | Energy metabolism\| |
| | | Glycolysis/gluconeogenesis |
| NSE_RS03630, NRI_RS03715, NHE_RS03795 | 2,3-bisphosphoglycerate- | Energy metabolism\| |
| | independent | Glycolysis/gluconeogenesis |
| | phosphoglycerate mutase | |
| NSE_RS01510, NRI_RS01560, NHE_RS01555 | pyruvate, phosphate | Energy metabolism\|Other |
| | dikinase | |
| NSE_RS00860, NRI_RS00900, NHE_RS00855 | ribose 5-phosphate | Energy metabolism\|Pentose |
| | isomerase B | phosphate pathway |
| NSE_RS01395, NRI_RS01445, NHE_RS01420 | ribulose-phosphate 3- | Energy metabolism\|Pentose |
| | epimerase | phosphate pathway |
| NSE_RS02860, NRI_RS02950, NHE_RS02985, | transketolase | Energy metabolism\|Pentose |
| NHE_RS02985 | | phosphate pathway |
| NSE_RS03100, NRI_RS03185, NHE_RS03250 | putative transaldolase | Energy metabolism\|Pentose |
| | | phosphate pathway |
| NSE_RS01865, NRI_RS01910, NHE_RS01915, | dihydrolipoamide | Energy metabolism\|Pyruvate |
| NHE_RS02820, NRI_RS02795 | dehydrogenase | dehydrogenase |
| NSE_RS02705, NRI_RS02795, NHE_RS02820, | dihydrolipoamide | Energy metabolism\|Pyruvate |
| NHE_RS01915, NRI_RS01910 | dehydrogenase | dehydrogenase |
| NSE_RS03030, NRI_RS03120, NHE_RS03185 | putative pyruvate | Energy metabolism\|Pyruvate |
| | dehydrogenase complex, | dehydrogenase |
| | E1 component, beta | |
| | subunit | |
| NSE_RS03255, NRI_RS03335, NHE_RS03420 | pyruvate dehydrogenase | Energy metabolism\|Pyruvate |
| | complex, E1 component, | dehydrogenase |
| | pyruvate dehydrogenase | |
| | alpha subunit | |
| NSE_RS00205, NRI_RS00190, NHE_RS00200 | succinate dehydrogenase, | Energy metabolism\|TCA cycle |
| | cytochrome b556 subunit | |
| NSE_RS00210, NRI_RS00195, NHE_RS00205 | putative succinate | Energy metabolism\|TCA cycle |
| | dehydrogenase, | |
| | hydrophobic membrane | |
| | anchor protein | |
| NSE_RS00250, NRI_RS00235, NHE_RS00245 | fumarate hydratase, class II | Energy metabolism\|TCA cycle |
| NSE_RS00670, NRI_RS00710, NHE_RS00655 | dehydrogenase, | Energy metabolism\|TCA cycle |
| | isocitrate/isopropylmalate | |
| | family | |
| NSE_RS00995, NRI_RS01035, NHE_RS00995 | succinyl-CoA synthetase, | Energy metabolism\|TCA cycle |
| | alpha subunit | |
| NSE_RS01000, NRI_RS01040, NHE_RS01000 | succinyl-CoA synthetase, | Energy metabolism\|TCA cycle |
| | beta subunit | |
| NSE_RS02185, NRI_RS02230, NHE_RS02265 | succinate dehydrogenase | Energy metabolism\|TCA cycle |
| | and tumarate reductase | |
| | iron-sulfur protein | |
| NSE_RS02255, NRI_RS02300, NHE_RS02335, | 2-oxoglutarate | Energy metabolism\|TCA cycle |
| NHE_RS04075, NRI_RS03955 | dehydrogenase, E2 | |
| | component, | |
| | dihydrolipoamide | |
| | succinyltransferase | |
| NSE_RS02370, NRI_RS02420, NHE_RS02465 | 2-oxoglutarate | Energy metabolism\|TCA cycle |
| | dehydrogenase, E1 | |
| | component | |
| NSE_RS02445, NRI_RS02500, NHE_RS02550 | aconitate hydratase 1 | Energy metabolism\|TCA cycle |
| NSE_RS02965, NRI_RS03055, NHE_RS03090 | citrate synthase | Energy metabolism\|TCA cycle |
| NSE_RS03875, NRI_RS03955, NHE_RS04075, | pyruvate dehydrogenase | Energy metabolism\|TCA cycle |
| NRI_RS02300, NHE_RS02335 | complex E2 component, | |
| | dihydrolipoamide | |
| | acetyltransferase | |
| NSE_RS03890, NRI_RS03970, NHE_RS04090 | malate dehydrogenase, | Energy metabolism\|TCA cycle |
| | NAD-dependent | |

Fatty acid and phospholipid metabolism

| | | |
|---|---|---|
| NSE_RS00055, NRI_RS00045, NHE_RS00060 | CDP-diacylglycerol-- | Fatty acid and phospholipid |
| | glycerol-3-phosphate 3- | metabolism\|Biosynthesis |
| | phosphatidyltransferase | |
| NSE_RS00185, NRI_RS00170, NHE_RS00180 | enoyl-(acyl-carrier-protein) | Fatty acid and phospholipid |
| | reductase | metabolism\|Biosynthesis |
| NSE_RS00675, NRI_RS00715, NHE_RS00660 | putative transporter | Fatty acid and phospholipid |
| | | metabolism\|Biosynthesis |
| NSE_RS00980, NRI_RS01020, NHE_RS00980 | putative CDP- | Fatty acid and phospholipid |
| | diacylglycerol--serine O- | metabolism\|Biosynthesis |
| | phosphatidyltransferase | |
| NSE_RS01650, NRI_RS01700, NHE_RS01700 | 1-acyl-sn-glycerol-3- | Fatty acid and phospholipid |
| | phosphate acyltransferase | metabolism\|Biosynthesis |
| | family protein | |
| NSE_RS01820, NRI_RS01860, NHE_RS01865 | acyl carrier protein | Fatty acid and phospholipid |
| | | metabolism\|Biosynthesis |
| NSE_RS01825, NRI_RS01865, NHE_RS01870 | 3-oxoacyl-(acyl-carrier- | Fatty acid and phospholipid |
| | protein) synthase II | metabolism\|Biosynthesis |
| NSE_RS02235, NRI_RS02280, NHE_RS02315 | enoyl-(acyl-carrier-protein) | Fatty acid and phospholipid |
| | reductase II | metabolism\|Biosynthesis |
| NSE_RS02565, NRI_RS02625, NHE_RS02680 | 3-oxoacyl-(acyl-carrier- | Fatty acid and phospholipid |
| | protein) synthase III | metabolism\|Biosynthesis |
| NSE_RS02570, NRI_RS02635, NHE_RS02685 | fatty acid phospholipid | Fatty acid and phospholipid |
| | synthesis protein PlsX | metabolism\|Biosynthesis |
| NSE_RS02675, NRI_RS02765, NHE_RS02785 | beta-hydroxyacyl-[acyl | Fatty acid and phospholipid |
| | carreir protein] dehydratase | metabolism\|Biosynthesis |
| | FabZ | |
| NSE_RS03840, NRI_RS03920, NHE_RS04035 | putative phosphatidate | Fatty acid and phospholipid |
| | cytidylyltransferase | metabolism\|Biosynthesis |
| NSE_RS03910, NRI_RS03990, NHE_RS04115 | malonyl CoA-acyl carrier | Fatty acid and phospholipid |
| | protein transacylase | metabolism\|Biosynthesis |
| NSE_RS03920, NRI_RS04000, NHE_RS04125 | holo-(acyl-carrier protein) | Fatty acid and phospholipid |
| | synthase | metabolism\|Biosynthesis |
| NSE_RS00900, NRI_RS00940, NHE_RS00895 | putative | Fatty acid and phospholipid |
| | phosphatidylglycerophosphatase A | metabolism\|Degradation |
| NSE_RS00970, NRI_RS01010, NHE_RS00970 | conserved hypothetical | Fatty acid and phospholipid |
| | protein | metabolism\|Degradation |
| NSE_RS01200, NRI_RS01245, NHE_RS01205 | phosphatidylglycerophosphatase A | Fatty acid and phospholipid |
| | | metabolism\|Degradation |
| NSE_RS03230, NRI_RS03310, NHE_RS03390 | propionyl-CoA | Fatty acid and phospholipid |
| | carboxylase, beta subunit | metabolism\|Degradation |
| NSE_RS03535, NRI_RS03615, NHE_RS03700 | patatin-like phospholipase | Fatty acid and phospholipid |
| | family protein | metabolism\|Degradation |

Protein Fate
Sec-dependent pathway:

| | | |
|---|---|---|
| NSE_RS02215, NRI_RS02260, NHE_RS02295 | signal recognition particle | Protein fate\|Protein and peptide |
| | protein SRP | secretion and trafficking |
| NSE_RS02240, NRI_RS02285, NHE_RS02320 | signal recognition particle- | Protein fate\|Protein and peptide |
| | docking protein FtsY | secretion and trafficking |
| NSE_RS00925, NRI_RS00965, NHE_RS00920 | preprotein translocase, | Protein fate\|Protein and peptide |
| | SecA subunit | secretion and trafficking |
| NSE_RS01475, NRI_RS01525, NHE_RS01515 | putative protein-export | Protein fate\|Protein and peptide |
| | protein SecB | secretion and trafficking |
| NSE_RS02765, NRI_RS02855, NHE_RS02885 | preprotein translocase, | Protein fate\|Protein and peptide |
| | SecE subunit | secretion and trafficking |
| NSE_RS01165, NRI_RS01210, NHE_RS01170 | preprotein translocase, | Protein fate\|Protein and peptide |
| | SecY subunit | secretion and trafficking |
| NSE_RS03565, NRI_RS03645, NHE_RS03730 | preprotein transiocase, | Protein fate\|Protein and peptide |
| | SecG subunit | secretion and trafficking |
| NSE_RS01700, NRI_RS01745, NHE_RS01745 | putative protein-export | Protein fate\|Protein and peptide |
| | membrane protein SecF | secretion and trafficking |
| NSE_RS02550, NRI_RS02610, NHE_RS02655 | protein-export membrane | Protein fate\|Protein and peptide |
| | protein SecD | secretion and trafficking |
| NSE_RS01325, NRI_RS01375, NHE_RS01335 | preprotein transiocase, | Protein fate\|Protein and peptide |
| | YajC subunit | secretion and trafficking |

Tat pathway:

| | | |
|---|---|---|
| NSE_RS01950, NRI_RS01995, NHE_RS02000 | twin-arginine translocation | Protein fate\|Protein and peptide |
| | protein, TatA/E family | secretion and trafficking |
| NSE_RS02090, NRI_RS02135, NHE_RS02160 | twin-arginine translocation | Protein fate\|Protein and peptide |
| | protein, TatB | secretion and trafficking |
| NSE_RS00495, NRI_RS00540, NHE_RS00490 | Sec-independent protein | Protein fate\|Protein and peptide |
| | translocase TatC | secretion and trafficking |

T1SS:

| | | |
|---|---|---|
| NSE_RS03825, NRI_RS03905, NHE_RS04020 | type I secretion membrane | Protein fate\|Protein and peptide |
| | fusion protein, HlyD | secretion and trafficking |
| | family | |
| NSE_RS00180, NRI_RS00165, NHE_RS00175 | type I secretion system | Protein fate\|Protein and peptide |
| | ATPase HlyB | secretion and trafficking |
| NSE_RS03240, NRI_RS03320, NHE_RS03400 | outer membrane efflux | Protein fate\|Protein and peptide |
| | protein TolC | secretion and trafficking\|\| |
| | | Transport and binding proteins\| |
| | | Unknown substrate |

T4SS:

| | | |
|---|---|---|
| NSE_RS03000, NRI_RS03090, NHE_RS03145 | type IV secretion system | Protein fate\|Protein and peptide |
| | protein VirD4 | secretion and trafficking |
| NSE_RS03005, NRI_RS03095, NHE_RS03150 | type IV secretion system | Protein fate\|Protein and peptide |
| | protein VirB11 | secretion and trafficking |
| NSE_RS03010, NRI_RS03100, NHE_RS03155 | type IV secretion system | Protein fate\|Protein and peptide |
| | protein VirB10 | secretion and trafficking |
| NSE_RS03015, NRI_RS03105, NHE_RS03160 | type IV secretion system | Protein fate\|Protein and peptide |
| | protein VirB9 (VirB9-1) | secretion and trafficking |
| NSE_RS03020, NRI_RS03110, NHE_RS03165 | type IV secretion system | Protein fate\|Protein and peptide |
| | protein VirB8 (VirB8-1) | secretion and trafficking |
| NSE_RS03120, NRI_RS03205, NHE_RS03270 | type IV secretion system | Protein fate\|Protein and peptide |
| | protein VirB4 (VirB4-2) | secretion and trafficking |
| NSE_RS03125, NRI_RS03210, NHE_RS03285 | type IV secretion system | Protein fate\|Protein and peptide |
| | protein VirB2 (VirB2-2) | secretion and trafficking |
| NSE_RS03130, NRI_RS03215, NHE_RS03285 | type IV secretion system | Protein fate\|Protein and peptide |
| | protein VirB2 (VirB2-1) | secretion and trafficking |
| NSE_RS00825, NRI_RS00865, NHE_RS00820 | type IV secretion system | Protein fate\|Protein and peptide |
| | protein VirB9 (VirB9-2) | secretion and trafficking |
| NSE_RS00830, NRI_RS00870, NHE_RS00825 | type IV secretion system | Protein fate\|Protein and peptide |
| | protein VirB8 (VirB8-2) | secretion and trafficking |
| NSE_RS03500, NRI_RS03580, NHE_RS03665 | type IV secretion system | Protein fate\|Protein and peptide |
| | protein, VirB6 family | secretion and trafficking |
| | (VirB6-4) | |
| NSE_RS03505, NRI_RS03585, NHE_RS03670 | type IV secretion system | Protein fate\|Protein and peptide |
| | protein, VirB6 family | secretion and trafficking |
| | (VirB6-3) | |
| NSE_RS03510, NRI_RS03590, NHE_RS03675 | type IV secretion system | Protein fate\|Protein and peptide |
| | protein, VirB6 family | secretion and trafficking |
| | (VirB6-2) | |
| NSE_RS03515, NRI_RS03595, NHE_RS03680 | type IV secretion system | Protein fate\|Protein and peptide |
| | protein VirB6 (VirB6-1) | secretion and trafficking |
| NSE_RS03520, NRI_RS03600, NHE_RS03685 | type IV secretion system | Protein fate\|Protein and peptide |
| | protein VirB4 (VirB4-1) | secretion and trafficking |
| NSE_RS04020, NRI_RS04090, NHE_RS03236 | type IV secretion system | Protein fate\|Protein and peptide |
| | protein VirB7 | secretion and trafficking |
| NSE_RS03525, NRI_RS03605, NHE_RS03690 | type IV secretion system | Protein fate\|Protein and peptide |
| | protein VirB3 | secretion and trafficking |

Chaperones:

| | | |
|---|---|---|
| NSE_RS02605, NRI_RS02675, NHE_RS02725 | 60 kDa chaperonin GroEL | Protein fate\|Protein folding and |
| | | stabilization |
| NSE_RS02610, NRI_RS02680, NHE_RS02730 | 10 kDa chaperomn GroES | Protein fate\|Protein folding and |
| | | stabilization |
| NSE_RS02190, NRI_RS02235, NHE_RS02270 | chaperone protein DnaJ | Protein fate\|Protein folding and |
| | | stabilization |
| NSE_RS03330, NRI_RS03410, NHE_RS03495 | DnaJ domain protein | Protein fate\|Protein folding and |
| | | stabilization |
| NSE_RS00085, NRI_RS00075, NHE_RS00095 | chaperone protein Dnak | Protein fate\|Protein folding and |
| | | stabilization |
| NSE_RS01235, NRI_RS01280, NHE_RS01240 | putative chaperone protein | Protein fate\|Protein folding and |
| | HscB | stabilization |
| NSE_RS00835, NRI_RS00875, NHE_RS00830 | co-chaperone GrpE | Protein fate\|Protein folding and |
| | | stabilization |
| NSE_RS02000, NRI_RS02040, NHE_RS02045 | heat shock protein HtpG | Protein fate\|Protein folding and |
| | | stabilization |
| NSE_RS01230, NRI_RS01275, NHE_RS01235 | putative chaperone protein | Protein fate\|Protein folding and |
| | HscA | stabilization |

Other functions:

| | | |
|---|---|---|
| NSE_RS00630, NRI_RS00670, NHE_RS00615, | HflK protein | Protein fate\|Degradation of |
| NHE_RS00620 | | proteins, peptides, and |
| | | glycopeptides |
| NSE_RS00635, NRI_RS00675, NHE_RS00620 | HflC protein | Protein fate\|Degradation of |
| | | proteins, peptides, and |
| | | glycopeptides |
| NSE_RS00680, NRI_RS00720, NHE_RS00670, | peptidase, M16 family | Protein fate\|Degradation of |
| NHE_RS03895, NRI_RS03805, | | proteins, peptides, and |
| NRI_RS03800, | | glycopeptides |
| NHE_RS03890 | | |
| NSE_RS00940, NRI_RS00980, NHE_RS00935 | putative | Protein fate\|Degradation of |
| | metalloendopeptidase, | proteins, peptides, and |
| | glycoprotease family | glycopeptides |
| NSE_RS01440, NRI_RS01490, NHE_RS01475 | ATP-dependent protease | Protein fate\|Degradation of |
| | La | proteins, peptides, and |
| | | glycopeptides |
| NSE_RS01660, NRI_RS01710, NHE_RS01710 | signal peptide peptidase | Protein fate\|Degradation of |
| | SppA, 36K type | proteins, peptides, and |
| | | glycopeptides |
| NSE_RS01720, NRI_RS01760, NHE_RS01760 | ATP-dependent | Protein fate\|Degradation of |
| | metalloprotease FtsH | proteins, peptides, and |
| | | glycopeptides |
| NSE_RS01900, NRI_RS01945, NHE_RS01950 | metallopeptidase, M24 | Protein fate\|Degradation of |
| | family | proteins, peptides, and |
| | | glycopeptides |
| NSE_RS01920, NRI_RS01965, NHE_RS01970 | cytosol aminopeptidase | Protein fate\|Degradation of |
| | | proteins, peptides, and |
| | | glycopeptides |
| NSE_RS02920, NRI_RS03010, NHE_RS03045 | putative membrane- | Protein fate\|Degradation of |
| | associated zinc | proteins, peptides, and |
| | metalloprotease | glycopeptides |
| NSE_RS03055, NRI_RS03145, NHE_RS03210 | ATP-dependent Clp | Protein fate\|Degradation of |
| | protease, proteolytic | proteins, peptides, and |
| | subunit ClpP | glycopeptides |
| NSE_RS03435, NRI_RS03515, NHE_RS03600 | glycoprotease family | Protein fate\|Degradation of |
| | protein | proteins, peptides, and |
| | | glycopeptides |
| NSE_RS03720, NRI_RS03800, NHE_RS03890, | peptidase, M16 family | Protein fate\|Degradation of |
| NHE_RS00670, NRI_RS00720 | | proteins, peptides, and |
| | | glycopeptides |
| NSE_RS03725, NRI_RS03805, NHE_RS03895 | peptidase, M16 family | Protein fate\|Degradation of |
| | | proteins, peptides, and |
| | | glycopeptides |
| NSE_RS03735, NRI_RS03815, NHE_RS03905 | putative carboxypeptidase | Protein fate\|Degradation of |
| | | proteins, peptides, and |
| | | glycopeptides |
| NSE_RS01310, NRI_RS01360, NHE_RS01315, | putative lipoprotein | Protein fate\|Protein and peptide |
| NHE_RS02955, NRI_RS02925, | releasing system ATP- | secretion and trafficking |
| NHE_RS01995, NRI_RS01990, | binding protein LolD | |
| NHE_RS03450, NRI_RS03360, | | |
| NRI_RS03610, NHE_RS02960 | | |
| NSE_RS01665, NRI_RS01715, NHE_RS01715, | putative ABC transporter, | Protein fate\|Protein and peptide |
| NRI_RS03610, NHE_RS03695, | ATP-binding/permease | secretion and trafficking |
| NHE_RS01315, NHE_RS02955, | protein | |
| NRI_RS03360, NHE_RS03450 | | |
| NSE_RS01945, NRI_RS01990, NHE_RS01995, | ABC transporter, ATP- | Protein fate\|Protein and peptide |
| NHE_RS03450, NRI_RS01360, NRI_RS01715, | binding protein | secretion and trafficking |
| NHE_RS03695 | | |
| NSE_RS02835, NRI_RS02925, NHE_RS02955, | ABC transporter, ATP- | Protein fate\|Protein and peptide |
| NRI_RS01990, NHE_RS03450, NRI_RS03360, | binding protein | secretion and trafficking |
| NHE_RS01315, NHE_RS01995, NRI_RS01360, | | |
| NHE_RS03695, NRI_RS03610 | | |
| NSE_RS02885, NRI_RS02975, NHE_RS03010 | conserved hypothetical | Protein fate\|Protein and peptide |
| | protein | secretion and trafficking |
| NSE_RS02915, NRI_RS03005, NHE_RS03040 | outer membrane protein, | Protein fate\|Protein and peptide |
| | OMP85 family | secretion and trafficking |
| NSE_RS03175, NRI_RS03260, NHE_RS03335 | signal peptidase I | Protein fate\|Protein and peptide |
| | | secretion and trafficking |
| NSE_RS03285, NRI_RS03360, NHE_RS03450, | putative phosphate ABC | Protein fate\|Protein and peptide |
| NRI_RS01990, NHE_RS01995, | transporter, ATP-binding | secretion and trafficking |
| NHE_RS02955, NRI_RS02925, | protein | |
| NHE_RS01315, NHE_RS03695, | | |
| NRI_RS01360, NRI_RS03610 | | |
| NSE_RS03530, NRI_RS03610, NHE_RS03695, | putative ABC transporter, | Protein fate\|Protein and peptide |
| NHE_RS01715, NHE_RS00175, | ATP-binding | secretion and trafficking |
| NRI_RS00165, NRI_RS01715, | protein/permease protein | |
| NHE_RS01995, NRI_RS02925, | | |
| NRI_RS03360, NHE_RS02955 | | |
| NSE_RS03730, NRI_RS03810, NHE_RS03900 | signal peptidase II | Protein fate\|Protein and peptide |
| | | secretion and trafficking |
| NSE_RS00455, NRI_RS00500, NHE_RS00450, | ClpB protein | Protein fate\|Protein folding and |
| NHE_RS01305, NHE_RS01305, | | stabilization |
| NRI_RS01350 | | |
| NSE_RS00640, NRI_RS00680, NHE_RS00625 | periplasmic serine | Protein fate\|Protein folding and |
| | protease, DO/DeqQ family | stabilization |
| NSE_RS00685, NRI_RS00725, NHE_RS00675 | heat shock protein HslVU, | Protein fate\|Protein folding and |
| | HslV subunit | stabilization |
| NSE_RS00690, NRI_RS00730, NHE_RS00680, | heat shock protein HslVU, | Protein fate\|Protein folding and |
| NHE_RS03215, NHE_RS03215, | ATPase subunit HslU | stabilization |
| NRI_RS03150, NRI_RS03150 | | |
| NSE_RS01300, NRI_RS01350, NHE_RS01305, | ATP-dependent Clp | Protein fate\|Protein folding and |
| NHE_RS00450, NHE_RS00450, | protease, ATP-binding | stabilization |
| NRI_RS00500 | subunit ClpA | |
| NSE_RS01410, NRI_RS04070, NHE_RS01440 | disulfide bond formation | Protein fate\|Protein folding and |
| | protein, DsbB family | stabilization |
| NSE_RS02620, NRI_RS02690, NHE_RS02740 | rotamase family protein | Protein fate\|Protein folding and |
| | | stabilization |
| NSE_RS03050, NRI_RS03140, NHE_RS03205 | putative trigger factor | Protein fate\|Protein folding and |
| | | stabilization |
| NSE_RS03060, NRI_RS03150, NHE_RS03215, | ATP-dependent Clp | Protein fate\|Protein folding and |
| NHE_RS00680, NHE_RS00680, | protease, ATP-binding | stabilization |
| NRI_RS00730 | subunit ClpX | |
| NSE_RS03470, NRI_RS03550, NHE_RS03630 | peptidyl-prolyl cis-trans | Protein fate\|Protein folding and |
| | isomerase, cyclophilin-type | stabilization |
| NSE_RS03600, NRI_RS03685, NHE_RS03765 | conserved hypothetical | Protein fate\|Protein folding and |
| | protein | stabilization |
| NSE_RS01400, NRI_RS01450, NHE_RS01425 | methionine | Protein fate\|Protein modification |
| | aminopeptidase, type I | and repair |
| NSE_RS01585, NRI_RS01635, NHE_RS01635 | peptide deformylase | Protein fate\|Protein modification |
| | | and repair |
| NSE_RS02010, NRI_RS02055, NHE_RS02065 | apolipoprotein N- | Protein fate\|Protein modification |
| | acyltransferase | and repair |
| NSE_RS02810, NRI_RS02900, NHE_RS02930 | biotin--acetyl-CoA- | Protein fate\|Protein modification |
| | carboxylase ligase | and repair |
| NSE_RS03485, NRI_RS03565, NHE_RS03645 | prolipoprotein | Protein fate\|Protein modification |
| | diacylglyceryl transferase | and repair |
| NSE_RS03680, NRI_RS03765, NHE_RS03845 | disulfide oxidoreductase | Protein fate\|Protein modification |
| | | and repair |
| NSE_RS00890, NRI_RS00930, NHE_RS00885, | TldD protein | Protein fate\|Other |
| NRI_RS02305 | | |
| NSE_RS02260, NRI_RS02305, NHE_RS02340, | pmbA protein | Protein fate\|Other |
| NHE_RS00885, NRI_RS00930 | | |

Protein synthesis

| | | |
|---|---|---|
| NSE_RS01270, NRI_RS01315, NHE_RS01275 | peptidyl-tRNA hydrolase | Protein synthesis\|Other |
| NSE_RS01795, NRI_RS01835, NHE_RS03870, | GTP-binding protein | Protein synthesis\|Other |
| NRI_RS03790 | Obg/CgtA | |
| NSE_RS03295, NRI_RS03375, NHE_RS03460 | SsrA-binding protein | Protein synthesis\|Other |
| NSE_RS03705, NRI_RS03790, NHE_RS03870, | GTP-binding protein YchF | Protein synthesis\|Other |
| NRI_RS01835 | | |
| NSE_RS00275, NRI_RS00265, NHE_RS00275 | ribosomal protein S18 | Protein synthesis\|Ribosomal |
| | | proteins: synthesis and |
| | | modification |
| NSE_RS00845, NRI_RS00885, NHE_RS00840 | ribosomal protein L35 | Protein synthesis\|Ribosomal |
| | | proteins: synthesis and |
| | | modification |
| NSE_RS00260, NRI_RS00250, NHE_RS00260 | ribosomal protein S15 | Protein synthesis\|Ribosomal |
| | | proteins: synthesis and |
| | | modification |
| NSE_RS00270, NRI_RS00260, NHE_RS00270 | ribosomal protein L9 | Protein synthesis\|Ribosomal |
| | | proteins: synthesis and |
| | | modification |
| NSE_RS00280, NRI_RS00270, NHE_RS00280 | ribosomal protein S6 | Protein synthesis\|Ribosomal |
| | | proteins: synthesis and |
| | | modification |
| NSE_RS00475, NRI_RS00520, NHE_RS00470 | ribosomal protein S16 | Protein synthesis\|Ribosomal |
| | | proteins: synthesis and |
| | | modification |
| NSE_RS00850, NRI_RS00890, NHE_RS00845 | ribosomal protein L20 | Protein synthesis\|Ribosomal |
| | | proteins: synthesis and |
| | | modification |
| NSE_RS01030, NRI_RS01070, NHE_RS01030 | ribosomal protein L33 | Protein synthesis\|Ribosomal |
| | | proteins: synthesis and |
| | | modification |
| NSE_RS01065, NRI_RS01110, NHE_RS01070 | ribosomal protein S10 | Protein synthesis\|Ribosomal |
| | | proteins: synthesis and |
| | | modification |
| NSE_RS01070, NRI_RS01115, NHE_RS01075 | ribosomal protein L3 | Protein synthesis\|Ribosomal |
| | | proteins: synthesis and |
| | | modification |
| NSE_RS01075, NRI_RS01120, NHE_RS01080 | ribosomal protein L4 | Protein synthesis\|Ribosomal |
| | | proteins: synthesis and |
| | | modification |
| NSE_RS01080, NRI_RS01125, NHE_RS01085 | ribosomal protein L23 | Protein synthesis\|Ribosomal |
| | | proteins: synthesis and |
| | | modification |
| NSE_RS01085, NRI_RS01130, NHE_RS01090 | ribosomal protein L2 | Protein synthesis\|Ribosomal |
| | | proteins: synthesis and |
| | | modification |
| NSE_RS01090, NRI_RS01135, NHE_RS01095 | ribosomal protein S19 | Protein synthesis\|Ribosomal |
| | | proteins: synthesis and |
| | | modification |
| NSE_RS01095, NRI_RS01140, NHE_RS01100 | ribosomal protein L22 | Protein synthesis\|Ribosomal |
| | | proteins: synthesis and |
| | | modification |
| NSE_RS01100, NRI_RS01145, NHE_RS01105 | ribosomal protein S3 | Protein synthesis\|Ribosomal |
| | | proteins: synthesis and |
| | | modification |
| NSE_RS01105, NRI_RS01150, NHE_RS01110 | ribosomal protein L16 | Protein synthesis\|Ribosomal |
| | | proteins: synthesis and |
| | | modification |
| NSE_RS01115, NRI_RS01160, NHE_RS01120 | ribosomal protein S17 | Protein synthesis\|Ribosomal |
| | | proteins: synthesis and |
| | | modification |
| NSE_RS01120, NRI_RS01165, NHE_RS01125 | ribosomal protein L14 | Protein synthesis\|Ribosomal |
| | | proteins: synthesis and |
| | | modification |
| NSE_RS01125, NRI_RS01170, NHE_RS01130 | ribosomal protein L24 | Protein synthesis\|Ribosomal |
| | | proteins: synthesis and |
| | | modification |
| NSE_RS01130, NRI_RS01175, NHE_RS01135 | ribosomal protein L5 | Protein synthesis\|Ribosomal |
| | | proteins: synthesis and |
| | | modification |
| NSE_RS01135, NRI_RS01180, NHE_RS01140 | ribosomal protein S14 | Protein synthesis\|Ribosomal |
| | | proteins: synthesis and |
| | | modification |
| NSE_RS01140, NRI_RS01185, NHE_RS01145 | ribosomal protein S8 | Protein synthesis\|Ribosomal |
| | | proteins: synthesis and |
| | | modification |
| NSE_RS01145, NRI_RS01190, NHE_RS01150 | ribosomal protein L6 | Protein synthesis\|Ribosomal |
| | | proteins: synthesis and |
| | | modification |
| NSE_RS01150, NHE_RS01155, NRI_RS01195 | ribosomal protein L18 | Protein synthesis\|Ribosomal |
| | | proteins: synthesis and |
| | | modification |
| NSE_RS01155, NRI_RS01200, NHE_RS01160 | ribosomal protein S5 | Protein synthesis\|Ribosomal |
| | | proteins: synthesis and |
| | | modification |
| NSE_RS01160, NRI_RS01205, NHE_RS01165 | ribosomal protein L15 | Protein synthesis\|Ribosomal |
| | | proteins: synthesis and |
| | | modification |
| NSE_RS01175, NRI_RS01220, NHE_RS01180 | ribosomal protein S13 | Protein synthesis\|Ribosomal |
| | | proteins: synthesis and |
| | | modification |
| NSE_RS01180, NRI_RS01225, NHE_RS01185 | ribosomal protein S11 | Protein synthesis\|Ribosomal |
| | | proteins: synthesis and |
| | | modification |
| NSE_RS01190, NRI_RS01235, NHE_RS01195 | ribosomal protein L17 | Protein synthesis\|Ribosomal |
| | | proteins: synthesis and |
| | | modification |
| NSE_RS01265, NRI_RS01310, NHE_RS01270 | ribosomal 5S rRNA E-loop | Protein synthesis\|Ribosomal |
| | binding protein | proteins: synthesis and |
| | Ctc/L25/TL5 | modification |
| NSE_RS01335, NRI_RS01385, NHE_RS01345 | ribosomal protein L28 | Protein synthesis\|Ribosomal |
| | | proteins: synthesis and |
| | | modification |
| NSE_RS01655, NRI_RS01705, NHE_RS01705 | ribosomal protein S1 | Protein synthesis\|Ribosomal |
| | | proteins: synthesis and |
| | | modification |
| NSE_RS02040, NRI_RS02085, NHE_RS02095 | conserved hypothetical | Protein synthesis\|Ribosomal |
| | protein | proteins: synthesis and |
| | | modification |
| NSE_RS02395, NRI_RS02450, NHE_RS02490 | ribosomal protein S4 | Protein synthesis\|Ribosomal |
| | | proteins: synthesis and |
| | | modification |
| NSE_RS02740, NRI_RS02830, NHE_RS02860 | ribosomal protein L7/L12 | Protein synthesis\|Ribosomal |
| | | proteins: synthesis and |
| | | modification |
| NSE_RS02745, NRI_RS02835, NHE_RS02865 | 50S ribosomal protein L10 | Protein synthesis\|Ribosomal |
| | | proteins: synthesis and |
| | | modification |
| NSE_RS02750, NRI_RS02840, NHE_RS02870 | ribosomal protein L1 | Protein synthesis\|Ribosomal |
| | | proteins: synthesis and |
| | | modification |
| NSE_RS02755, NRI_RS02845, NHE_RS02875 | ribosomal protein L11 | Protein synthesis\|Ribosomal |
| | | proteins: synthesis and |
| | | modification |
| NSE_RS02785, NRI_RS02875, NHE_RS02905 | ribosomal protein S7 | Protein synthesis\|Ribosomal |
| | | proteins: synthesis and |
| | | modification |
| NSE_RS02790, NRI_RS02880, NHE_RS02910 | ribosomal protein S12 | Protein synthesis\|Ribosomal |
| | | proteins: synthesis and |
| | | modification |
| NSE_RS03210, NRI_RS03295, NHE_RS03370 | ribosomal protein S20 | Protein synthesis\|Ribosomal |
| | | proteins: synthesis and |
| | | modification |
| NSE_RS03370, NRI_RS03450, NHE_RS03550 | ribosomal protein S21 | Protein synthesis\|Ribosomal |
| | | proteins: synthesis and |
| | | modification |
| NSE_RS03410, NRI_RS03490, NHE_RS03575 | ribosomal protein S9 | Protein synthesis\|Ribosomal |
| | | proteins: synthesis and |
| | | modification |
| NSE_RS03415, NRI_RS03495, NHE_RS03580 | ribosomal protein L13 | Protein synthesis\|Ribosomal |
| | | proteins: synthesis and |
| | | modification |
| NSE_RS03650, NRI_RS03735, NHE_RS03815 | ribosomal protein L19 | Protein synthesis\|Ribosomal |
| | | proteins: synthesis and |
| | | modification |
| NSE_RS03660, NRI_RS03745, NHE_RS03825 | ribosomal protein L27 | Protein synthesis\|Ribosomal |
| | | proteins: synthesis and |
| | | modification |
| NSE_RS03665, NRI_RS03750, NHE_RS03830 | ribosomal protein L21 | Protein synthesis\|Ribosomal |
| | | proteins: synthesis and |
| | | modification |
| NSE_RS00765, NRI_RS00805, NHE_RS00760 | translation elongation | Protein synthesis\|Translation |
| | factor P | factors |
| NSE_RS01205, NRI_RS01250, NHE_RS01210 | translation initiation factor | Protein synthesis\|Translation |
| | IF-3 | factors |
| NSE_RS01425, NRI_RS01475, NHE_RS01460 | ribosomal subunit interface | Protein synthesis\|Translation |
| | protein | factors |
| NSE_RS01645, NRI_RS01695, NHE_RS01695, | peptide chain release factor 1 | Protein synthesis\|Translation |
| NHE_RS02565 | | factors |
| NSE_RS02130, NRI_RS02175, NHE_RS02205, | translation initiation factor | Protein synthesis\|Translation |
| NRI_RS03220, NHE_RS03290, | IF-2 | factors |
| NRI_RS02805, | | |
| NHE_RS02895, NRI_RS02865 | | |
| NSE_RS02715, NRI_RS02805, NHE_RS02835, | GTP-binding protein TypA | Protein synthesis\|Translation |
| NHE_RS03290, NRI_RS03220, | | factors |
| NHE_RS02895, NRI_RS02865, | | |
| NHE_RS02900, NRI_RS02870, | | |
| NRI_RS02870, NRI_RS02175, NHE_RS02205 | | |
| NSE_RS02775, NRI_RS02865, NHE_RS02895, | translation elongation | Protein synthesis\|Translation |
| NRI_RS02805, NHE_RS02835, | factor Tu | factors |
| NRI_RS03220, | | |
| NRI_RS02175 | | |
| NSE_RS02780, NRI_RS02870, NHE_RS02900, | translation elongation | Protein synthesis\|Translation |
| NHE_RS02835, NHE_RS02835, | factor G | factors |
| NRI_RS02805, NRI_RS02805, | | |
| NHE_RS03290, NHE_RS03290, | | |
| NRI_RS03220, NRI_RS03220 | | |
| NSE_RS03135, NRI_RS03220, NHE_RS03290, | GTP-binding protein LepA | Protein synthesis\|Translation |
| NHE_RS02835, NRI_RS02805, | | factors |
| NHE_RS02900, NHE_RS02900, | | |
| NRI_RS02870, NRI_RS02870, | | |
| NHE_RS02205, NHE_RS02895, NRI_RS02175 | | |
| NSE_RS03595, NRI_RS03675, NHE_RS03760 | translation initiation factor | Protein synthesis\|Translation |
| | IF-1 | factors |
| NSE_RS03850, NRI_RS03930, NHE_RS04045 | ribosome recycling factor | Protein synthesis\|Translation |
| | | factors |
| NSE_RS03860, NRI_RS03940, NHE_RS04055 | translation elongation | Protein synthesis\|Translation |
| | factor Ts | factors |
| NSE_RS00215, NRI_RS00200, NHE_RS00210 | arginyl-tRNA synthetase | Protein synthesis\|tRNA |
| | | aminoacylation |
| NSE_RS00360, NRI_RS00355, NHE_RS00370 | glutamyl-tRNA(Gln) | Protein synthesis\|tRNA |
| | amidotransferase, B | aminoacylation |
| | subunit | |
| NSE_RS00385, NRI_RS00385, NHE_RS00385 | methionyl-tRNA | Protein synthesis\|tRNA |
| | formyltransferase | aminoacylation |
| NSE_RS00600, NRI_RS00640, NHE_RS00585 | alanyl-tRNA synthetase | Protein synthesis\|tRNA |
| | | aminoacylation |
| NSE_RS00840, NRI_RS00880, NHE_RS00835 | tryptophanyl-tRNA | Protein synthesis\|tRNA |
| | synthetase | aminoacylation |
| NSE_RS00855, NRI_RS00895, NHE_RS00850 | phenylalanyl-tRNA | Protein synthesis\|tRNA |
| | synthetase, alpha subunit | aminoacylation |
| NSE_RS01025, NRI_RS01065, NHE_RS01025 | glutamyl-tRNA(Gln) | Protein synthesis\|tRNA |
| | amidotransferase, A | aminoacylation |
| | subunit | |
| NSE_RS01210, NRI_RS01255, NHE_RS01215 | threonyl-tRNA synthetase | Protein synthesis\|tRNA |
| | | aminoacylation |
| NSE_RS01380, NRI_RS01430, NHE_RS01390 | cysteinyl-tRNA synthetase | Protein synthesis\|tRNA |
| | | aminoacylation |
| NSE_RS01430, NRI_RS01480, NHE_RS01465 | tyrosyl-tRNA synthetase | Protein synthesis\|tRNA |
| | | aminoacylation |
| NSE_RS01500, NRI_RS01550, NHE_RS01540, | prolyl-tRNA synthetase | Protein synthesis\|tRNA |
| NHE_RS01215 | | aminoacylation |
| NSE_RS01725, NRI_RS01765, NHE_RS01765 | putative phenylalanyl- | Protein synthesis\|tRNA |
| | tRNA synthetase, beta | aminoacylation |
| | subunit | |
| NSE_RS01930, NRI_RS01975, NHE_RS01980 | aspartyl-tRNA synthetase | Protein synthesis\|tRNA |
| | | aminoacylation |
| NSE_RS02055, NRI_RS02100, NHE_RS02110, | leucyl-tRNA synthetase | Protein synthesis\|tRNA |
| NHE_RS02560, NRI_RS02510 | | aminoacylation |
| NSE_RS02100, NRI_RS02145, NHE_RS02170 | putative glutamyl- | Protein synthesis\|tRNA |
| | tRNA(Gln) | aminoacylation |
| | amidotransferase, C | |
| | subunit | |
| NSE_RS02110, NHE_RS02180, NHE_RS03125, | glutamyl-tRNA synthetase | Protein synthesis\|tRNA |
| NRI_RS03080 | | aminoacylation |
| NSE_RS02200, NRI_RS02245, NHE_RS02280, | isoleucyl-tRNA synthetase | Protein synthesis\|tRNA |
| NRI_RS02510, NHE_RS02560 | | aminoacylation |
| NSE_RS02335, NRI_RS02385, NHE_RS02425 | seryl-tRNA synthetase | Protein synthesis\|tRNA |
| | | aminoacylation |
| NSE_RS02455, NRI_RS02510, NHE_RS02560, | putative valyl-tRNA | Protein synthesis\|tRNA |
| NHE_RS02560, NHE_RS02280, | synthetase | aminoacylation |
| NRI_RS02245, NRI_RS02100, | | |
| NHE_RS02110 | | |
| NSE_RS02990, NRI_RS03080, NHE_RS03125, | glutamyl-tRNA synthetase | Protein synthesis\|tRNA |
| NHE_RS02180 | | aminoacylation |
| NSE_RS03075, NRI_RS03160, NHE_RS03225 | glycyl-tRNA synthetase, | Protein synthesis\|tRNA |
| | beta subunit | aminoacylation |
| NSE_RS03080, NRI_RS03165, NHE_RS03230 | glycyl-tRNA synthetase, | Protein synthesis\|tRNA |
| | alpha subunit | aminoacylation |
| NSE_RS03140, NRI_RS03225, NHE_RS03295 | lysyl-tRNA synthetase | Protein synthesis\|tRNA |
| | | aminoacylation |
| NSE_RS03160, NRI_RS03245, NHE_RS03315 | histidyl-tRNA synthetase | Protein synthesis\|tRNA |
| | | aminoacylation |
| NSE_RS03640, NRI_RS03725, NHE_RS03805 | methionyl-tRNA | Protein synthesis\|tRNA |
| | synthetase | aminoacylation |
| NSE_RS00080, NRI_RS00070, NHE_RS00085 | queuine tRNA- | Protein synthesis\|tRNA and |
| | ribosyltransferase | rRNA base modification |
| NSE_RS00090, NRI_RS00080, NHE_RS00100 | tRNA pseudouridine | Protein synthesis\|tRNA and |
| | synthase A | rRNA base modification |
| NSE_RS00405, NRI_RS00410, NHE_RS00410 | tRNA pseudouridine | Protein synthesis\|tRNA and |
| | synthase B | rRNA base modification |
| NSE_RS00570, NRI_RS00610, NHE_RS00535, | ribosomal large subunit | Protein synthesis\|tRNA and |
| NHE_RS02370, NRI_RS02330 | pseudouridine synthases, | rRNA base modification |
| | RluA family | |
| NSE_RS01050, NRI_RS01095, NHE_RS01055 | RNA methyltransferase, | Protein synthesis\|tRNA and |
| | TrmH family, group 3 | rRNA base modification |
| NSE_RS01485, NRI_RS01535, NHE_RS01525 | dimethyladenosine | Protein synthesis\|tRNA and |
| | transferase | rRNA base modification |
| NSE_RS01890, NRI_RS01935, NHE_RS01940 | tRNA (5- | Protein synthesis\|tRNA and |
| | methylaminomethyl-2- | rRNA base modification |
| | thiouridylate)- | |
| | methyltransferase | |
| NSE_RS02245, NRI_RS02290, NHE_RS02325 | ribosomal RNA large | Protein synthesis\|tRNA and |
| | subunit methyltransferase J | rRNA base modification |
| NSE_RS02285, NRI_RS02330, NHE_RS02370, | ribosomal large subunit | Protein synthesis\|tRNA and |
| NHE_RS00535, NRI_RS00610 | pseudouridine synthase C | rRNA base modification |
| NSE_RS02340, NRI_RS02390, NHE_RS02430 | tRNA delta(2)- | Protein synthesis\|tRNA and |
| | isopentenylpyrophosphate | rRNA base modification |
| | transferase | |
| NSE_RS02870, NRI_RS02960, NHE_RS02995 | glucose inhibited division | Protein synthesis\|tRNA and |
| | protein A | rRNA base modification |
| NSE_RS03655, NRI_RS03740, NHE_RS03820 | tRNA (guanine-N1)- | Protein synthesis\|tRNA and |
| | methyltransferase | rRNA base modification |
| NSE_RS03870, NRI_RS03950, NHE_RS04065 | ubiquinone/menaquinone | Protein synthesis\|tRNA and |
| | biosynthesis | rRNA base modification |
| | methlytransferase UbiE | |

Purines, pyrimidines, nucleosides, and nucleotides biosynthesis

| | | |
|---|---|---|
| NSE_RS00625, NRI_RS00665, NHE_RS00610 | thymidylate synthase, | Purines, pyrimidines, nucleosides, |
| | flavin-dependent | and nucleotides\|2′- |
| | | Deoxyribonucleotide metabolism |
| NSE_RS01670, NRI_RS01720, NHE_RS01720 | ribonucleoside-diphosphate | Purines, pyrimidines, nucleosides, |
| | reductase, alpha subunit | and nucleotides\|2′- |
| | | Deoxyribonucleotide metabolism |
| NSE_RS02120, NRI_RS02165, NHE_RS02190 | ribonucleoside-diphosphate | Purines, pyrimidines, nucleosides, |
| | reductase, beta subunit | and nucleotides\|2′- |
| | | Deoxyribonucleotide metabolism |
| NSE_RS03895, NRI_RS03975, NHE_RS04095 | deoxyuridine | Purines, pyrimidines, nucleosides, |
| | 5′triphosphate | and nucleotides\|2′- |
| | nucleotidohydrolase | Deoxyribonucleotide metabolism |
| NSE_RS03930, NRI_RS04010, NHE_RS04140 | putative deoxycytidine | Purines, pyrimidines, nucleosides, |
| | triphosphate deaminase | and nucleotides\|2′- |
| | | Deoxyribonucleotide metabolism |
| NSE_RS01170, NRI_RS01215, NHE_RS01175 | adenylate kinase | Purines, pyrimidines, nucleosides, |
| | | and nucleotides\|Nucleotide and |
| | | nucleoside interconversions |
| NSE_RS01850, NRI_RS01890, NHE_RS01895 | putative | Purines, pyrimidines, nucleosides, |
| | deoxyguanosinetriphosphate | and nucleotides\|Nucleotide and |
| | triphosphohydrolase | nucleoside interconversions |
| NSE_RS02250, NRI_RS02295, NHE_RS02330 | thymidylate kinase | Purines, pyrimidines, nucleosides, |
| | | and nucleotides\|Nucleotide and |
| | | nucleoside interconversions |
| NSE_RS02300, NRI_RS02345, NHE_RS02390 | nucleoside diphosphate | Purines, pyrimidines, nucleosides, |
| | kinase | and nucleotides\|Nucleotide and |
| | | nucleoside interconversions |
| NSE_RS02950, NRI_RS03040, NHE_RS03075 | guanylate kinase | Purines, pyrimidines, nucleosides, |
| | | and nucleotides\|Nucleotide and |
| | | nucleoside interconversions |
| NSE_RS03855, NRI_RS03935, NHE_RS04050 | uridylate kinase | Purines, pyrimidines, nucleosides, |
| | | and nucleotides\|Nucleotide and |
| | | nucleoside interconversions |
| NSE_RS00130, NRI_RS00115, NHE_RS00125 | phosphoribosylformylglycinamidine | Purines, pyrimidines, nucleosides, |
| | cyclo-ligase | and nucleotides\|Purine |
| | | ribonucleotide biosynthesis |
| NSE_RS00265, NRI_RS00255, NHE_RS00265 | adenylosuccinate lyase | Purines, pyrimidines, nucleosides, |
| | | and nucleotides\|Purine |
| | | ribonucleotide biosynthesis |
| NSE_RS00725, NRI_RS00760, NHE_RS00715 | phosphoribosylaminoimidazolecarboxamide | Purines, pyrimidines, nucleosides, |
| | formyltransferase/IMP | and nucleotides\|Purine |
| | cyclohydrolase | ribonucleotide biosynthesis |
| NSE_RS00755, NRI_RS00795, NHE_RS00750, | amidophosphoribosyltransferase | Purines, pyrimidines, nucleosides, |
| NHE_RS02150 | | and nucleotides\|Purine |
| | | ribonucleotide biosynthesis |
| NSE_RS00895, NRI_RS00935, NHE_RS00890 | adenylosuccinate | Purines, pyrimidines, nucleosides, |
| | synthetase | and nucleotides\|Purine |
| | | ribonucleotide biosynthesis |
| NSE_RS00935, NRI_RS00975, NHE_RS00930 | phosphoribosylaminoimidazole | Purines, pyrimidines, nucleosides, |
| | carboxylase, catalytic | and nucleotides\|Purine |
| | subunit | ribonucleotide biosynthesis |
| NSE_RS01445, NRI_RS01495, NHE_RS01480 | conserved hypothetical | Purines, pyrimidines, nucleosides, |
| | protein | and nucleotides\|Purine |
| | | ribonucleotide biosynthesis |
| NSE_RS01810, NRI_RS01850, NHE_RS01855 | putative | Purines, pyrimidines, nucleosides, |
| | phosphoribosylformylglycinamidine | and nucleotides\|Purine |
| | synthase I | ribonucleotide biosynthesis |
| NSE_RS01915, NRI_RS01960, NHE_RS01965 | phosphoribosylglycinamide | Purines, pyrimidines, nucleosides, |
| | formyltransferase | and nucleotides\|Purine |
| | | ribonucleotide biosynthesis |
| NSE_RS02145, NRI_RS02190, NHE_RS02220 | inosine-5′-monophosphate | Purines, pyrimidines, nucleosides, |
| | dehydrogenase | and nucleotides\|Purine |
| | | ribonucleotide biosynthesis |
| NSE_RS03300, NRI_RS03380, NHE_RS03465 | putative | Purines, pyrimidines, nucleosides, |
| | phosphoribosylformylglycinamidine | and nucleotides\|Purine |
| | synthase II | ribonucleotide biosynthesis |
| NSE_RS03320, NRI_RS03400, NHE_RS03485 | ribose-phosphate | Purines, pyrimidines, nucleosides, |
| | pyrophosphokinase | and nucleotides\|Purine |
| | | ribonucleotide biosynthesis |
| NSE_RS03475, NRI_RS03555, NHE_RS03635 | phosphoribosylaminoimidazole- | Purines, pyrimidines, nucleosides, |
| | succinocarboxamide | and nucleotides\|Purine |
| | synthase | ribonucleotide biosynthesis |
| NSE_RS03610, NRI_RS03695, NHE_RS03775 | GMP synthase | Purines, pyrimidines, nucleosides, |
| | | and nucleotides\|Purine |
| | | ribonucleotide biosynthesis |
| NSE_RS03770, NRI_RS03855, NHE_RS03945 | phosphoribosylamine-- | Purines, pyrimidines, nucleosides, |
| | glycine ligase | and nucleotides\|Purine |
| | | ribonucleotide biosynthesis |
| NSE_RS03935, NRI_RS04015, NHE_RS04150 | phosphoribosylaminoimidazole | Purines, pyrimidines, nucleosides, |
| | carboxylase, ATPase | and nucleotides\|Purine |
| | subunit | ribonucleotide biosynthesis |
| NSE_RS00595, NRI_RS00635, NHE_RS00580 | dihydroorotase, | Purines, pyrimidines, nucleosides, |
| | multifunctional complex | and nucleotides\|Pyrimidine |
| | type | ribonucleotide biosynthesis |
| NSE_RS00700, NRI_RS00740, NHE_RS00690 | dihydroorotate | Purines, pyrimidines, nucleosides, |
| | dehydrogenase | and nucleotides\|Pyrimidine |
| | | ribonucleotide biosynthesis |
| NSE_RS00880, NRI_RS00920, NHE_RS00875 | carbamoyl-phosphate | Purines, pyrimidines, nucleosides, |
| | synthase, large subunit | and nucleotides\|Pyrimidine |
| | | ribonucleotide biosynthesis |
| NSE_RS02035, NRI_RS02080, NHE_RS02090 | carbamoyl-phosphate | Purines, pyrimidines, nucleosides, |
| | synthase, small subunit | and nucleotides\|Pyrimidine |
| | | ribonucleotide biosynthesis |
| NSE_RS02075, NRI_RS02120, NHE_RS02130 | aspartate | Purines, pyrimidines, nucleosides, |
| | carbamoyltransferase | and nucleotides\|Pyrimidine |
| | | ribonucleotide biosynthesis |
| NSE_RS02205, NRI_RS02250, NHE_RS02285 | orotate | Purines, pyrimidines, nucleosides, |
| | phosphoribosyltransferase | and nucleotides\|Pyrimidine |
| | | ribonucleotide biosynthesis |
| NSE_RS03215, NRI_RS03300, NHE_RS03380 | orotidine 5′-phosphate | Purines, pyrimidines, nucleosides, |
| | decarboxylase | and nucleotides\|Pyrimidine |
| | | ribonucleotide biosynthesis |
| NSE_RS03570, NRI_RS03650, NHE_RS03735 | CTP synthase | Purines, pyrimidines, nucleosides, |
| | | and nucleotides\|Pyrimidine |
| | | ribonucleotide biosynthesis |

Regulatory functions

| | | |
|---|---|---|
| NSE_RS00025, NRI_RS00015, NHE_RS00035 | sensor histidine kinase | Regulatory functions\|Protein |
| | PleC | interactions |
| NSE_RS02175, NRI_RS02220, NHE_RS02255 | Sensor histidine kinase, | Regulatory functions\|Protein |
| | PleC-like | interactions |
| NSE_RS02085, NRI_RS02130, NHE_RS02155 | response regulator/GGDEF | Regulatory functions\|Other |
| | domain protein PleD | |
| NSE_RS01495, NRI_RS01545, NHE_RS01535 | Sensor histidine | Regulatory functions\|Protein |
| | kinase/response regulator, | interactions |
| | CckA | |
| NSE_RS00930, NRI_RS00970, NHE_RS00925 | DNA-binding response | Regulatory functions\|DNA |
| | regulator CtrA | interactions |
| NSE_RS01785, NRI_RS01825, NHE_RS01830 | EAL domain protein | Regulatory functions\|Other |
| NSE_RS03985, NRI_RS02020, NHE_RS04205 | Transposase and | Regulatory functions\|Other |
| | inactivated derivatives | |
| NSE_RS01195, NRI_RS01240, NHE_RS01200 | transcriptional regulator, | Regulatory functions\|DNA |
| | MerR family protein | interactions |
| NSE_RS01460, NRI_RS01510, NHE_RS01495 | ATP cone domain protein | Regulatory functions\|DNA |
| | | interactions |
| NSE_RS03325, NRI_RS03405, NHE_RS03490 | NifU-like domain protein | Regulatory functions\|Other |

Transcription

| | | |
|---|---|---|
| NSE_RS01295, NRI_RS01345, NHE_RS01300 | RNA polymerase sigma | Transcription\|Transcription |
| | factor RpoD | factors |
| NSE_RS01415, NRI_RS01465, NHE_RS01445 | RNA polymerase sigma-32 | Transcription\|Transcription |
| | factor RpoH | factors |
| NSE_RS00160, NRI_RS00145, NHE_RS00155 | Neorickettsia expression | Transcription\|Transcription |
| | regulator NhxR | factors |
| NSE_RS00920, NRI_RS00960, NHE_RS00915 | putative transcriptional | Transcription\|Transcription |
| | regulator Tr1 | factors |
| NSE_RS02065, NRI_RS02110, NHE_RS02120 | SOS-response | Unknown function\|General |
| | transcriptional repressor | |
| | LexA | |
| NSE_RS02690, NRI_RS02780, NHE_RS02805 | ribonuclease HI | Transcription\|Degradation of |
| | | RNA |
| NSE_RS02850, NRI_RS02940, NHE_RS02970 | ribonuclease HII | Transcription\|Degradation of |
| | | RNA |
| NSE_RS01185, NRI_RS01230, NHE_RS01190 | DNA-directed RNA | Transcription\|DNA-dependent |
| | polymerase, alpha subunit | RNA polymerase |
| NSE_RS02735, NRI_RS02825, NHE_RS02855 | DNA-directed RNA | Transcription\|DNA-dependent |
| | polymerase, beta subunit | RNA polymerase |
| NSE_RS03975, NRI_RS00150, NHE_RS00160 | DNA-directed RNA | Transcription\|DNA-dependent |
| | polymerase, omega subunit | RNA polymerase |
| NSE_RS03380, NRI_RS03460, NHE_RS03560 | metallo-beta-lactamase | Transcription\|Other |
| | family, beta-CASP | |
| | subfamily | |
| NSE_RS00480, NRI_RS00525, NHE_RS00475 | putative 16S rRNA | Transcription\|RNA processing |
| | processing protein RimM | |
| NSE_RS02135, NRI_RS02180, NHE_RS02210 | putative ribosome-binding | Transcription\|RNA processing |
| | factor A | |
| NSE_RS02165, NRI_RS02210, NHE_RS02245 | 3′-5′ exonuclease family | Transcription\|RNA processing |
| | protein | |
| NSE_RS03495, NRI_RS03575, NHE_RS04225 | ribonuclease P protein | Transcription\|RNA processing |
| | component | |
| NSE_RS03745, NRI_RS03830, NHE_RS03915 | ribonuclease III | Transcription\|RNA processing |
| NSE_RS00305, NRI_RS00295, NHE_RS00310 | transcription termination | Transcription\|Transcription |
| | factor Rho | factors |
| NSE_RS02125, NRI_RS02170, NHE_RS02200 | N utilization substance | Transcription\|Transcription |
| | protein A | factors |
| NSE_RS02450, NRI_RS02505, NHE_RS02555 | conserved hypothetical | Transcription\|Transcription |
| | protein | factors |
| NSE_RS02700, NRI_RS02790, NHE_RS02815 | transcription elongation | Transcription\|Transcription |
| | factor GreA | factors |
| NSE_RS02760, NRI_RS02850, NHE_RS02880 | putative transcription | Transcription\|Transcription |
| | termination/antitermination | factors |
| | factor NusG | |
| NSE_RS03585, NRI_RS03665, NHE_RS03750 | putative N utilization | Transcription\|Transcription |
| | substance protein B | factors |

Transport and binding proteins

| | | |
|---|---|---|
| NSE_RS01435, NRI_RS01485, NHE_RS01470 | bacterioferritin | Transport and binding proteins\| |
| | | Cations and iron carrying |
| | | compounds |
| NSE_RS00590, NRI_RS00630, NHE_RS00575 | sodium:alanine symporter | Transport and binding proteins\| |
| | family protein | Amino acids, peptides and amines |
| NSE_RS02940, NRI_RS03030, NHE_RS03065 | putative sodium:proline | Transport and binding proteins\| |
| | symporter | Amino acids, peptides and amines |
| NSE_RS00285, NRI_RS00275, NHE_RS00285 | putative phosphate ABC | Transport and binding proteins\| |
| | transporter, periplasmic | Anions |
| | phosphate-binding protein | |
| NSE_RS00800, NRI_RS00840, NHE_RS00795, | phosphate ABC | Transport and binding proteins\| |
| NHE_RS01990, NRI_RS01985 | transporter, permease | Anions |
| | protein PstC | |
| NSE_RS01940, NRI_RS01985, NHE_RS01990, | phosphate ABC | Transport and binding proteins\| |
| NRI_RS00840, NHE_RS00795 | transporter, permease | Anions |
| | protein PstA | |
| NSE_RS00035, NRI_RS00025, NHE_RS00045 | Fe(3+) ABC transporter | Transport and binding proteins\| |
| | substrate-binding protein | Cations and iron carrying |
| | | compounds |
| NSE_RS00135, NRI_RS00120, NHE_RS00130 | Na(+)/H(+) antiporter | Transport and binding proteins\| |
| | subunit C | Cations and iron carrying |
| | | compounds |
| NSE_RS00140, NRI_RS00125, NHE_RS00135 | multisubunit Na+/H+ | Transport and binding proteins\| |
| | antiporter, MnhB subunit | Cations and iron carrying |
| | | compounds |
| NSE_RS00145, NRI_RS00130, NHE_RS00140 | multisubunit Na+/H+ | Transport and binding proteins\| |
| | antiporter, MnhB subunit | Cations and iron carrying |
| | | compounds |
| NSE_RS00150, NRI_RS00135, NHE_RS00145 | monovalent cation/proton | Transport and binding proteins\| |
| | antiporter, MnhG/PhaG | Cations and iron carrying |
| | subunit | compounds |
| NSE_RS01875, NRI_RS01920, NHE_RS01920 | magnesium transporter | Transport and binding proteins\| |
| | | Cations and iron carrying |
| | | compounds |
| NSE_RS03605, NRI_RS03690, NHE_RS03770 | glutathione-regulated | Transport and binding proteins\| |
| | potassium-efflux system | Cations and iron carrying |
| | protein | compounds |
| NSE_RS00535, NRI_RS00580, NHE_RS00530 | putative permease | Transport and binding proteins\| |
| | | Other |
| NSE_RS02070, NRI_RS02115, NHE_RS02125 | heme exporter protein, | Transport and binding proteins\| |
| | CcmC family | Other |
| NSE_RS00585, NRI_RS00625, NHE_RS00570 | efflux transporter, RND | Transport and binding proteins\| |
| | family, MFP subunit | Unknown substrate |
| NSE_RS00605, NRI_RS00645, NHE_RS00590 | putative transporter | Transport and binding proteins\| |
| | | Unknown substrate |
| NSE_RS00715, NRI_RS00755, NHE_RS00705 | Multiple resistance and pH | Transport and binding proteins\| |
| | regulation protein | Unknown substrate |
| | (MrpF/PhaF) | |
| NSE_RS00745, NRI_RS00785, NHE_RS00740 | permease, PerM family | Transport and binding proteins\| |
| | | Unknown substrate |
| NSE_RS00775, NRI_RS00815, NHE_RS00770 | putative transporter | Transport and binding proteins\| |
| | | Unknown substrate |
| NSE_RS00945, NRI_RS00985, NHE_RS00940 | TRAP transporter solute | Transport and binding proteins\| |
| | receptor, TAXI family | Unknown substrate |
| NSE_RS01260, NRI_RS01305, NHE_RS01265 | putative transporter | Transport and binding proteins\| |
| | | Unknown substrate |
| NSE_RS01285, NRI_RS01330, NHE_RS01290 | putative ATP-NAD kinase | Transport and binding proteins\| |
| | | Unknown substrate |
| NSE_RS01615, NRI_RS01665, NHE_RS01665 | ATP synthase F0, B′ chain | Transport and binding proteins\| |
| | | Unknown substrate |
| NSE_RS02225, NRI_RS02270, NHE_RS02305 | RDD family protein | Transport and binding proteins\| |
| | | Unknown substrate |
| NSE_RS02830, NRI_RS02920, NHE_RS02950 | putative ABC transporter, | Transport and binding proteins\| |
| | permease protein | Unknown substrate |
| NSE_RS02840, NRI_RS02930, NHE_RS02960, | ABC transporter, ATP- | Transport and binding proteins\| |
| NHE_RS02960 | binding protein | Unknown substrate |
| NSE_RS03170, NRI_RS03255, NHE_RS03325 | major facilitator family | Transport and binding proteins\| |
| | transporter | Unknown substrate |
| NSE_RS03310, NRI_RS03390, NHE_RS03475 | putative permease | Transport and binding proteins\| |
| | | Unknown substrate |
| NSE_RS03425, NRI_RS03505, NHE_RS03590 | TRAP transporter, | Transport and binding proteins\| |
| | 4TM/12TM fusion protein | Unknown substrate |
| NSE_RS03445, NRI_RS03525, NHE_RS03605 | putative membrane protein | Transport and binding proteins\| |
| | | Unknown substrate |
| NSE_RS03450, NRI_RS03530, NHE_RS03610 | mechanosensitive ion | Transport and binding proteins\| |
| | channel family protein | Unknown substrate |

Unknown functions

| | | |
|---|---|---|
| NSE_RS02155, NRI_RS02200, NHE_RS02235 | mce-related protein | Unclassified\|Role category not |
| | | yet assigned |
| NSE_RS00195, NRI_RS00180, NHE_RS00190 | hexapeptide transferase | Unknown function\|Enzymes of |
| | family protein | unknown specificity |
| NSE_RS00735, NRI_RS00775, NHE_RS00730 | conserved hypothetical | Unknown function\|Enzymes of |
| | protein | unknown specificity |
| NSE_RS00785, NRI_RS00825, NHE_RS00780 | putative methyltransferase | Unknown function\|Enzymes of |
| | | unknown specificity |
| NSE_RS01320, NRI_RS01370, NHE_RS01330 | aminomethyl transferase | Unknown function\|Enzymes of |
| | family protein | unknown specificity |
| NSE_RS01470, NRI_RS01520, NHE_RS01510 | hydrolase, TatD family | Unknown function\|Enzymes of |
| | | unknown specificity |
| NSE_RS01565, NRI_RS01615, NHE_RS01615 | NADH-ubiquinone | Unknown function\|Enzymes of |
| | oxidoreductase family | unknown specificity |
| | protein | |
| NSE_RS01905, NRI_RS01950, NHE_RS01955 | putative hydrolase | Unknown function\|Enzymes of |
| | | unknown specificity |
| NSE_RS02095, NRI_RS02140, NHE_RS02165 | NAD-glutamate | Unknown function\|Enzymes of |
| | dehydrogenase family | unknown specificity |
| | protein | |
| NSE_RS02230, NRI_RS02275, NHE_RS02310 | S-adenosylmethionine- | Unknown function\|Enzymes of |
| | dependent | unknown specificity |
| | methyltransferases | |
| NSE_RS02380, NRI_RS02430, NHE_RS02475 | metallo-beta-lactamase | Unknown function\|Enzymes of |
| | family protein | unknown specificity |
| NSE_RS02440, NRI_RS02495, NHE_RS02545 | O-methyltransferase family | Unknown function\|Enzymes of |
| | protein | unknown specificity |
| NSE_RS02660, NRI_RS02750, NHE_RS02770 | acetyltransferase, GNAT | Unknown function\|Enzymes of |
| | family | unknown specificity |
| NSE_RS02695, NRI_RS02785, NHE_RS02810 | conserved hypothetical | Unknown function\|Enzymes of |
| | protein | unknown specificity |
| NSE_RS03280, NRI_RS03355, NHE_RS03440 | flavin reductase family | Unknown function\|Enzymes of |
| | protein | unknown specificity |
| NSE_RS03355, NRI_RS03435, NHE_RS03530 | Ser/Thr protein | Unknown function\|Enzymes of |
| | phosphatase family protein | unknown specificity |
| NSE_RS03545, NRI_RS03625, NHE_RS03710 | hydrolase, alpha/beta fold | Unknown function\|Enzymes of |
| | family | unknown specificity |
| NSE_RS03800, NRI_RS03880, NHE_RS03995 | HAD-superfamily | Unknown function\|Enzymes of |
| | hydrolase, subfamily IA, | unknown specificity |
| | variant 1 | |
| NSE_RS02935, NRI_RS03025, NHE_RS03060 | Alkyl hydroperoxide | Transport and binding proteins\| |
| | reductase subunit AhpC | Cations and iron carrying |
| | (bacterioferritin | compounds |
| | comigratory protein) | |
| NSE_RS00075, NRI_RS00065, NHE_RS00080 | ankyrin repeat protein | Unknown function\|General |
| NSE_RS00485, NRI_RS00530, NHE_RS00480 | RmuC domain protein | Unknown function\|General |
| NSE_RS00500, NRI_RS00545, NHE_RS00495 | modification methylase, | Unknown function\|General |
| | HemK family | |
| NSE_RS00505, NRI_RS00550, NHE_RS00500 | hypothetical protein | Unknown function\|General |
| NSE_RS00790, NRI_RS00830, NHE_RS00785 | BolA family protein | Unknown function\|General |
| NSE_RS00795, NRI_RS00835, NHE_RS00790 | glutaredoxin-related | Unknown function\|General |
| | protein | |
| NSE_RS00955, NRI_RS00995, NHE_RS00955 | putative membrane protein | Unknown function\|General |
| NSE_RS01020, NRI_RS01060, NHE_RS01020 | rhodanese domain protein | Unknown function\|General |
| NSE_RS01315, NRI_RS01365, NHE_RS01325, | ComEC/Rec2 family | Unknown function\|General |
| NHE_RS04175 | protein | |
| NSE_RS01345, NRI_RS01395, NHE_RS01355 | aromatic rich family | Unknown function\|General |
| | protein | |
| NSE_RS01360, NRI_RS01410, NHE_RS01370 | CBS/transporter associated | Unknown function\|General |
| | domain protein | |
| NSE_RS01525, NRI_RS01575, NHE_RS01575 | putative tRNA- | Unknown function\|General |
| | dihydrouridine synthase | |
| NSE_RS01600, NRI_RS01650, NHE_RS01650 | YihY family protein | Unknown function\|General |
| NSE_RS01640, NRI_RS01690, NHE_RS01690 | CBS domain protein | Unknown function\|General |
| NSE_RS01680, NRI_RS01730, NHE_RS01730 | HIT domain protein | Unknown function\|General |
| NSE_RS02005, NRI_RS02050, NHE_RS02050, | ankyrin repeat protein | Unknown function\|General |
| NRI_RS02045, NHE_RS02055 | | |
| NSE_RS02105, NRI_RS02150, NHE_RS02175 | hypothetical protein | Unknown function\|General |
| NSE_RS02195, NRI_RS02240, NHE_RS02275, | putative GTP-binding | Unknown function\|General |
| NRI_RS03000, NHE_RS03035, | protein EngA | |
| NHE_RS03035 | | |
| NSE_RS02375, NRI_RS02425, NHE_RS02470 | Sua5/YciO/YrdC/YwlC | Unknown function\|General |
| | family protein | |
| NSE_RS02425, NRI_RS02480, NHE_RS02530 | fructose-1,6- | Unknown function\|General |
| | bisphosphatase, class II | |
| NSE_RS02910, NRI_RS03000, NHE_RS03035, | tRNA modification | Unknown function\|General |
| NHE_RS03035, NRI_RS02240, | GTPase TrmE | |
| NHE_RS02275, NHE_RS02275 | | |
| NSE_RS02945, NRI_RS03035, NHE_RS03070 | pentapeptide repeat domain | Unknown function\|General |
| | protein | |
| NSE_RS03095, NRI_RS03180, NHE_RS03245 | conserved hypothetical | Unknown function\|General |
| | protein | |
| NSE_RS03145, NRI_RS03230, NHE_RS03300 | hypothetical protein | Unknown function\|General |
| NSE_RS03165, NRI_RS03250, NHE_RS03320 | BolA family protein | Unknown function\|General |
| NSE_RS03250, NRI_RS03330, NHE_RS03410 | inositol monophosphatase | Unknown function\|General |
| | family protein | |
| NSE_RS03420, NRI_RS03500, NHE_RS03585 | CvpA family protein | Unknown function\|General |
| NSE_RS03460, NRI_RS03540, NHE_RS03620 | class II aldolase/adducin | Unknown function\|General |
| | domain protein | |
| NSE_RS03615, NRI_RS03700, NHE_RS03780 | ATP-binding protein, | Unknown function\|General |
| | Mrp/Nbp35 family | |
| NSE_RS03750, NRI_RS03835, NHE_RS03920 | iojap-related protein | Unknown function\|General |
| NSE_RS03835, NRI_RS03915, NHE_RS04030 | conserved hypothetical | Unknown function\|General |
| | protein | |
| NSE_RS03980, NRI_RS00695, NHE_RS04170 | Smr domain protein | Unknown function\|General |
| NSE_RS00020, NRI_RS00010, NHE_RS00030 | hypothetical protein | Hypothetical proteins\|Conserved |
| NSE_RS00030, NRI_RS00020, NHE_RS00040 | hypothetical protein | Hypothetical proteins\|Conserved |
| NSE_RS00040, NRI_RS00030, NHE_RS00050 | conserved hypothetical | Hypothetical proteins\|Conserved |
| | protein | |
| NSE_RS00105, NRI_RS00090, NHE_RS00105 | Ankyrin-repeat protein | Hypothetical proteins\|Conserved |
| NSE_RS00110, NRI_RS00100, NHE_RS00110 | hypothetical protein | Hypothetical proteins\|Conserved |
| NSE_RS00115, NRI_RS00105, NHE_RS00115 | hypothetical protein | Hypothetical proteins\|Conserved |
| NSE_RS00125, NRI_RS00110, NHE_RS00120 | putative membrane protein | Hypothetical proteins\|Conserved |
| NSE_RS00155, NRI_RS00140, NHE_RS00150 | conserved hypothetical | Hypothetical proteins\|Conserved |
| | protein | |
| NSE_RS00175, NRI_RS00160, NHE_RS00170 | hypothetical protein | Hypothetical proteins\|Conserved |
| NSE_RS00230, NRI_RS00215, NHE_RS00225 | conserved hypothetical | Hypothetical proteins\|Conserved |
| | protein | |
| NSE_RS00490, NRI_RS00535, NHE_RS00485 | conserved hypothetical | Hypothetical proteins\|Conserved |
| | protein | |
| NSE_RS00710, NRI_RS00750, NHE_RS00700 | hypothetical protein | Hypothetical proteins\|Conserved |
| NSE_RS00740, NRI_RS00780, NHE_RS00735 | conserved hypothetical | Hypothetical proteins\|Conserved |
| | protein | |
| NSE_RS00770, NRI_RS00810, NHE_RS00765 | putative lipoprotein | Hypothetical proteins\|Conserved |
| NSE_RS00805, NRI_RS00845, NHE_RS00800 | hypothetical protein | Hypothetical proteins\|Conserved |
| NSE_RS00960, NRI_RS01000, NHE_RS00960 | conserved hypothetical | Hypothetical proteins\|Conserved |
| | protein | |
| NSE_RS00990, NRI_RS01030, NHE_RS00990 | conserved hypothetical | Hypothetical proteins\|Conserved |
| | protein TIGR00043 | |
| NSE_RS01220, NRI_RS01265, NHE_RS01225 | hypothetical protein | Hypothetical proteins\|Conserved |
| NSE_RS01305, NRI_RS01355, NHE_RS01310 | hypothetical protein | Hypothetical proteins\|Conserved |
| NSE_RS01350, NRI_RS01400, NHE_RS01360 | conserved hypothetical | Hypothetical proteins\|Conserved |
| | protein | |
| NSE_RS01365, NRI_RS01415, NHE_RS04180 | hypothetical protein | Hypothetical proteins\|Conserved |
| NSE_RS01375, NRI_RS01425, NHE_RS01385 | conserved hypothetical | Hypothetical proteins\|Conserved |
| | protein | |
| NSE_RS01450, NRI_RS01500, NHE_RS01485 | putative membrane protein | Hypothetical proteins\|Conserved |
| NSE_RS01480, NRI_RS01530, NHE_RS01520 | Tim44-like domain protein | Hypothetical proteins\|Conserved |
| NSE_RS01490, NRI_RS01540, NHE_RS01530 | hypothetical protein | Hypothetical proteins\|Conserved |
| NSE_RS01520, NRI_RS01570, NHE_RS01570 | hypothetical protein | Hypothetical proteins\|Conserved |
| NSE_RS01540, NRI_RS01590, NHE_RS01590 | hypothetical protein | Hypothetical proteins\|Conserved |
| NSE_RS01575, NRI_RS01625, NHE_RS01625 | conserved hypothetical | Hypothetical proteins\|Conserved |
| | protein | |
| NSE_RS01625, NRI_RS01675, NHE_RS01675 | hypothetical protein | Hypothetical proteins\|Conserved |
| NSE_RS01735, NRI_RS01775, NHE_RS01775 | hypothetical protein | Hypothetical proteins\|Conserved |
| NSE_RS01775, NRI_RS01815, NHE_RS01820 | hypothetical protein | Hypothetical proteins\|Conserved |
| NSE_RS01780, NRI_RS01820, NHE_RS01825 | hypothetical protein | Hypothetical proteins\|Conserved |
| NSE_RS01815, NRI_RS01855, NHE_RS01860 | hypothetical protein | Hypothetical proteins\|Conserved |
| NSE_RS01835, NRI_RS01875, NHE_RS01880 | conserved hypothetical | Hypothetical proteins\|Conserved |
| | protein | |
| NSE_RS01840, NRI_RS01880, NHE_RS01885 | Protein of unknown | Hypothetical proteins\|Conserved |
| | function (DUF3442) | |
| NSE_RS01860, NRI_RS01905, NHE_RS01905 | conserved hypothetical | Hypothetical proteins\|Conserved |
| | protein | |
| NSE_RS01985, NRI_RS02025, NHE_RS02030 | conserved hypothetical | Hypothetical proteins\|Conserved |
| | protein TIGR00103 | |
| NSE_RS02015, NRI_RS02060, NHE_RS02070 | hypothetical protein | Hypothetical proteins\|Conserved |
| NSE_RS02050, NRI_RS02095, NHE_RS02105 | conserved hypothetical | Hypothetical proteins\|Conserved |
| | protein | |
| NSE_RS02080, NRI_RS02125, NHE_RS02135, | hypothetical protein | Hypothetical proteins\|Conserved |
| NHE_RS02140 | | |
| NSE_RS02115, NRI_RS02160, NHE_RS02185 | conserved hypothetical | Hypothetical proteins\|Conserved |
| | protein | |
| NSE_RS02140, NRI_RS02185, NHE_RS02215 | conserved domain protein | Hypothetical proteins\|Conserved |
| NSE_RS02150, NRI_RS02195, NHE_RS02230 | hypothetical protein | Hypothetical proteins\|Conserved |
| NSE_RS02160, NRI_RS02205, NHE_RS02240 | hypothetical protein | Hypothetical proteins\|Conserved |
| NSE_RS02210, NRI_RS02255, NHE_RS02290 | hypothetical protein | Hypothetical proteins\|Conserved |
| NSE_RS02315, NRI_RS02365, NHE_RS02405 | hypothetical protein | Hypothetical proteins\|Conserved |
| NSE_RS02320, NRI_RS02370, NHE_RS02410 | hypothetical protein | Hypothetical proteins\|Conserved |
| NSE_RS02345, NRI_RS02395, NHE_RS02435 | hypothetical protein | Hypothetical proteins\|Conserved |
| NSE_RS02385, NRI_RS02435, NHE_RS02480 | conserved hypothetical | Hypothetical proteins\|Conserved |
| | protein | |
| NSE_RS02420, NRI_RS02475, NHE_RS02525 | hypothetical protein | Hypothetical proteins\|Conserved |
| NSE_RS02435, NRI_RS02490, NHE_RS02540 | hypothetical protein | Hypothetical proteins\|Conserved |
| NSE_RS02490, NRI_RS02550, NHE_RS02595 | hypothetical protein | Hypothetical proteins\|Conserved |
| NSE_RS02575, NRI_RS02645, NHE_RS02695 | conserved domain protein | Hypothetical proteins\|Conserved |
| NSE_RS02590, NRI_RS02660, NHE_RS02710 | conserved hypothetical | Hypothetical proteins\|Conserved |
| | protein | |
| NSE_RS02600, NRI_RS02670, NHE_RS02720 | conserved hypothetical | Hypothetical proteins\|Conserved |
| | protein TIGR01033 | |
| NSE_RS02615, NRI_RS02685, NHE_RS02735 | hypothetical protein | Hypothetical proteins\|Conserved |
| NSE_RS02630, NRI_RS02700, NHE_RS02750 | hypothetical protein | Hypothetical proteins\|Conserved |
| NSE_RS02665, NRI_RS02755, NHE_RS02775 | hypothetical protein | Hypothetical proteins\|Conserved |
| NSE_RS02680, NRI_RS02770, NHE_RS02790 | OmpH-like outer | Hypothetical proteins\|Conserved |
| | membrane protein | |
| NSE_RS02710, NRI_RS02800, NHE_RS02830 | hypothetical protein | Hypothetical proteins\|Conserved |
| NSE_RS02845, NRI_RS02935, NHE_RS02965 | conserved hypothetical | Hypothetical proteins\|Conserved |
| | protein TIGR00150 | |
| NSE_RS02855, NRI_RS02945, NHE_RS02975 | hypothetical protein | Hypothetical proteins\|Conserved |
| NSE_RS02875, NRI_RS02965, NHE_RS03000 | hypothetical protein | Hypothetical proteins\|Conserved |
| NSE_RS02880, NRI_RS02970, NHE_RS03005 | conserved hypothetical | Hypothetical proteins\|Conserved |
| | protein | |
| NSE_RS02955, NRI_RS04080, NHE_RS03080 | hypothetical protein | Hypothetical proteins\|Conserved |
| NSE_RS02960, NRI_RS03050, NHE_RS03085 | hypothetical protein | Hypothetical proteins\|Conserved |
| NSE_RS02970, NRI_RS03060, NHE_RS03100 | hypothetical protein | Hypothetical proteins\|Conserved |
| NSE_RS03035, NRI_RS03125, NHE_RS03190 | hypothetical protein | Hypothetical proteins\|Conserved |
| NSE_RS03040, NRI_RS03130, NHE_RS03195 | hypothetical protein | Hypothetical proteins\|Conserved |
| NSE_RS03115, NRI_RS03200, NHE_RS03265 | hypothetical protein | Hypothetical proteins\|Conserved |
| NSE_RS03185, NRI_RS03270, NHE_RS03345 | hypothetical protein | Hypothetical proteins\|Conserved |
| NSE_RS03190, NRI_RS03275, NHE_RS03350 | hypothetical protein | Hypothetical proteins\|Conserved |
| NSE_RS03195, NRI_RS03280, NHE_RS03355 | hypothetical protein | Hypothetical proteins\|Conserved |
| NSE_RS03205, NRI_RS03290, NHE_RS03365 | hypothetical protein | Hypothetical proteins\|Conserved |
| NSE_RS03235, NRI_RS03315, NHE_RS03395 | hypothetical protein | Hypothetical proteins\|Conserved |
| NSE_RS03180, NSE_RS03390, NRI_RS03465, | tRNA-i(6)A37 | Hypothetical proteins\|Conserved |
| NHE_RS03565, NRI_RS03265, | modification enzyme MiaB | |
| NHE_RS03340 | | |
| NSE_RS03540, NRI_RS03620, NHE_RS03705 | hypothetical protein | Hypothetical proteins\|Conserved |
| NSE_RS03580, NRI_RS03660, NHE_RS03745 | hypothetical protein | Hypothetical proteins\|Conserved |
| NSE_RS03590, NRI_RS03670, NHE_RS03755 | maf protein | Hypothetical proteins\|Conserved |
| NSE_RS03625, NRI_RS03710, NHE_RS03790 | conserved hypothetical | Hypothetical proteins\|Conserved |
| | protein | |
| NSE_RS03635, NRI_RS03720, NHE_RS03800 | conserved hypothetical | Hypothetical proteins\|Conserved |
| | protein | |
| NSE_RS03675, NRI_RS03760, NHE_RS03840 | conserved hypothetical | Hypothetical proteins\|Conserved |
| | protein | |
| NSE_RS03685, NRI_RS03770, NHE_RS03850 | hypothetical protein | Hypothetical proteins\|Conserved |
| NSE_RS03740, NHE_RS03910, NRI_RS03820, | hypothetical protein | Hypothetical proteins\|Conserved |
| NRI_RS03825 | | |
| NSE_RS03815, NRI_RS03895, NHE_RS04010 | conserved hypothetical | Hypothetical proteins\|Conserved |
| | protein | |
| NSE_RS03820, NRI_RS03900, NHE_RS04015 | conserved hypothetical | Hypothetical proteins\|Conserved |
| | protein | |
| NSE_RS03925, NRI_RS04005, NHE_RS04130 | hypothetical protein | Hypothetical proteins\|Conserved |
| NSE_RS03940, NRI_RS04020, NHE_RS04155 | conserved domain protein | Hypothetical proteins\|Conserved |
| NSE_RS03965, NRI_RS04045, NHE_RS00020 | conserved hypothetical | Hypothetical proteins\|Conserved |
| | protein | |

¹Ortholog clusters were constructed using reciprocal BLASTP algorithm with E-value <1e−10, and grouped by functional role categories. The protein name and role category of the ortholog cluster are based on those from N. helminthoeca genome.

TABLE 6
N. helminthoeca-specific proteins compared to N. sennetsu and N. risticii¹

| | | | | | Top Hits Species |
|---|---|---|---|---|---|
| Locus_ID | Protein Name | AA# | Main Role | Sub Role | (Class, E-value) ² |
| NHE_RS00250 | aspartate kinase domain | 397 | Amino acid | Aspartate | Bacillus muralis |
| | protein | | biosynthesis | family | (Bacilli, 3e⁻⁵⁵) |
| NHE_RS02825 | succinyl-diaminopimelate | 371 | Amino acid | Aspartate | Wolbachia sp. of |
| | desuccinylase | | biosynthesis | family | Drosophila simulans |
| | | | | | (α-Proteobacteria, |
| | | | | | 1e⁻⁹⁴) |
| NHE_RS03445 | dihydrodipicolinate | 151 | Amino acid | Aspartate | Campylobacter |
| | reductase, family protein | | biosynthesis | family | ureolyticus (ε- |
| | | | | | Proteobacteria, 2e⁻²⁰) |
| NHE_RS03415 | magnesium chelatase, | 1049 | Biosynthesis | Chlorophyll | |
| | subunit ChlI family protein | | of cofactors | and | |
| | | | and | bacteriochlorphyll | |
| | | | prosthetic | | |
| | | | groups | | |
| NHE_RS00710 | UDP-N-acetylglucosamine | 432 | Cell | Biosynthesis | Paracoccus |
| | diphosphorylase | | envelope | and | tibetensis (α- |
| | (glucosamine-1-phosphate | | | degradation of | Proteobacteria, 4e⁻¹²²) |
| | N-acetyltransferase) | | | murein | |
| | | | | sacculus and | |
| | | | | peptidoglycan | |
| NHE_RS03095 | phosphoglucosamine | 439 | Cell | Biosynthesis | Magnetospirillum |
| | mutase | | envelope | and | marisnigri (α- |
| | | | | degradation of | Proteobacteria, 2e⁻¹⁴³) |
| | | | | murein | |
| | | | | sacculus and | |
| | | | | peptidoglycan | |
| NHE_RS01455 | penicillin binding | 528 | Cell | Biosynthesis | Ca. Neoehrlichia |
| | transpeptidase domain | | envelope | and | lotoris (α- |
| | protein | | | degradation of | Proteobacteria, 2e⁻¹²⁶) |
| | | | | murein | |
| | | | | sacculus and | |
| | | | | peptidoglycan | |
| NHE_RS03220 | rod shape-determining | 256 | Cell | Biosynthesis | |
| | MreC family protein | | envelope | and | |
| | | | | degradation of | |
| | | | | murein | |
| | | | | sacculus and | |
| | | | | peptidoglycan | |
| NHE_RS02450 | D-alanyl-D-alanine | 399 | Cell | Biosynthesis | Wolbachia sp. of |
| | carboxypeptidase family | | envelope | and | Cimex lectularius |
| | protein | | | degradation of | (α-Proteobacteria, e⁻¹¹⁴) |
| | | | | murein | |
| | | | | sacculus and | |
| | | | | peptidoglycan | |
| NHE_RS02495 | D-ala D-ala ligase family | 313 | Cell | Biosynthesis | Wolbachia sp. of |
| | protein | | envelope | and | Cimex lectularius |
| | | | | degradation of | (α-Proteobacteria, 5e⁻⁶⁰) |
| | | | | murein | |
| | | | | sacculus and | |
| | | | | peptidoglycan | |
| NHE_RS03375 | UDP-N-acetylmuramate-- | 424 | Cell | Biosynthesis | Thermodesulfovibrio |
| | alanine ligase | | envelope | and | sp. N1 |
| | | | | degradation of | (Nitrospirales, 2e⁻⁷⁹) |
| | | | | murein | |
| | | | | sacculus and | |
| | | | | peptidoglycan | |
| NHE_RS04135 | UDP-N-acetylglucosamine 1- | 418 | Cell | Biosynthesis | Ca. Pelagibacter sp. |
| | carboxyvinyltransferase | | envelope | and | IMCC9063 (α- |
| | | | | degradation of | Proteobacteria, 3e⁻¹⁰⁹) |
| | | | | murein | |
| | | | | sacculus and | |
| | | | | peptidoglycan | |
| NHE_RS02795 | phospho-N- | 324 | Cell | Biosynthesis | Bacillus bataviensis |
| | acetylmuramoyl- | | envelope | and | (Bacilli, 4e⁻⁶⁸) |
| | pentapeptide-transferase | | | degradation of | |
| | | | | murein | |
| | | | | sacculus and | |
| | | | | peptidoglycan | |
| NHE_RS02980 | D-alanyl-D-alanine | 284 | Cell | Biosynthesis | Crenothrix |
| | carboxypeptidase family | | envelope | and | polyspora (γ- |
| | protein | | | degradation of | Proteobacteria, 8e⁻⁶⁸) |
| | | | | murein | |
| | | | | sacculus and | |
| | | | | peptidoglycan | |
| NHE_RS00945 | UDP-N-acetylmuramoylalanine-- | 468 | Cell | Biosynthesis | Robiginitomaculum |
| | D-glutamate ligase | | envelope | and | antarcticum (α- |
| | | | | degradation of | Proteobacteria, 3e⁻⁵⁷) |
| | | | | murein | |
| | | | | sacculus and | |
| NHE_RS00380 | undecaprenyldiphospho- | 338 | Cell | Biosynthesis | Caedibacter |
| | muramoylpentapeptide β- | | envelope | and | varicaedens (α- |
| | N-acetyl-glucosaminyl- | | | degradation of | Proteobacteria, 3e⁻⁵⁷) |
| | transferase | | | murein | |
| | | | | sacculus and | |
| | | | | peptidoglycan | |
| NHE_RS03115 | penicillin binding | 566 | Cell | Biosynthesis | Anaplasma |
| | transpeptidase domain | | envelope | and | marginale (α- |
| | protein | | | degradation of | Proteobacteria, 2e⁻¹⁰⁸) |
| | | | | murein | |
| | | | | sacculus and | |
| | | | | peptidoglycan | |
| NHE_RS03175 | diaminopimelate | 266 | Cell | Biosynthesis | Prochlorococcus |
| | epimerase DapF | | envelope | and | marinus |
| | | | | degradation of | (Synechococcales, 9e⁻³⁰) |
| | | | | murein | |
| | | | | sacculus and | |
| | | | | peptidoglycan | |
| NHE_RS03990 | D-alanine | 310 | Cell | Biosynthesis | Ralstonia |
| | aminotransferase | | envelope | and | solanacearum (β- |
| | | | | degradation of | Proteobacteria, 1e⁻⁵⁸) |
| | | | | murein | |
| | | | | sacculus and | |
| | | | | peptidoglycan | |
| NHE_RS01430 | putative membrane | 164 | Cell | Other | |
| | protein | | envelope | | |
| NHE_RS04200 | rare lipoA family protein | 226 | Cell | Other | Wolbachia pipientis |
| | | | envelope | | (α-Proteobacteria, 1e⁻⁴³) |
| NHE_RS01810 | cell division protein FtsW | 369 | Cellular | Cell division | Caedibacter |
| | | | processes | | varicaedens (α- |
| | | | | | Proteobacteria, 4e⁻⁹¹) |
| NHE_RS02385 | rod shape-determining | 377 | Cellular | Cell division | Rhodospirillaceae |
| | protein RodA | | processes | | bacterium BRH_c57 |
| | | | | | (α-Proteobacteria, 4e⁻⁷⁷) |
| NHE_RS02100 | hydroxyacylglutathione | 247 | Cellular | Detoxification | Vibrio halioticoli (γ- |
| | hydrolase | | processes | | Proteobacteria, 2e⁻⁶⁷) |
| NHE_RS00090 | carbonic anhydrase family | 213 | Central | Other | Desulfovibrio |
| | protein | | intermediary | | vulgaris (δ- |
| | | | metabolism | | Proteobacteria, 1e⁻⁶²) |
| NHE_RS01115 | ribosomal protein L29 | 63 | Protein | Ribosomal | |
| | | | synthesis | proteins: | |
| | | | | synthesis and | |
| | | | | modification | |
| NHE_RS03975 | ribosonial L36 family | 44 | Protein | Ribosomal | |
| | protein | | synthesis | proteins: | |
| | | | | synthesis and | |
| | | | | modification | |
| NHE_RS03330 | putative Mg chelatase-like | 116 | Transport | Unknown | |
| | domain protein | | and binding | substrate | |
| | | | proteins | | |
| NHE_RS00420 | hypothetical protein | 257 | Hypothetical | General | |
| | | | proteins | | |
| NHE_RS02660 | hypothetical protein | 401 | Hypothetical | General | |
| | | | proteins | | |
| NHE_RS02195 | hypothetical protein | 59 | Hypothetical | General | |
| | | | proteins | | |
| NHE_RS00350 | hypothetical protein | 208 | Hypothetical | General | |
| | | | proteins | | |
| NHE_RS01395 | hypothetical protein | 60 | Hypothetical | General | |
| | | | proteins | | |
| NHE_RS03520 | hypothetical protein | 403 | Hypothetical | General | |
| | | | proteins | | |
| NHE_RS00320 | hypothetical protein | 272 | Hypothetical | General | |
| | | | proteins | | |
| NHE_RS03885 | hypothetical protein | 70 | Hypothetical | General | |
| | | | proteins | | |
| NHE_RS02765 | hypothetical protein | 133 | Hypothetical | General | |
| | | | proteins | | |
| NHE_RS00425 | hypothetical protein | 286 | Hypothetical | General | |
| | | | proteins | | |
| NHE_RS00665 | hypothetical protein | 292 | Hypothetical | General | |
| | | | proteins | | |
| NHE_RS02365 | hypothetical protein | 434 | Hypothetical | General | |
| | | | proteins | | |
| NHE_RS00355 | hypothetical protein | 118 | Hypothetical | General | |
| | | | proteins | | |
| NHE_RS01500 | hypothetical protein | 287 | Hypothetical | General | |
| | | | proteins | | |
| NHE_RS03525 | hypothetical protein | 601 | Hypothetical | General | |
| | | | proteins | | |
| NHE_RS00325 | hypothetical protein | 201 | Hypothetical | General | |
| | | | proteins | | |
| NHE_RS03925 | hypothetical protein | 99 | Hypothetical | General | |
| | | | proteins | | |
| NHE_RS03130 | hypothetical protein | 60 | Hypothetical | General | |
| | | | proteins | | |
| NHE_RS00430 | hypothetical protein | 91 | Hypothetical | General | |
| | | | proteins | | |
| NHE_RS00725 | hypothetical protein | 138 | Hypothetical | General | |
| | | | proteins | | |
| NHE_RS00360 | hypothetical protein | 98 | Hypothetical | General | |
| | | | proteins | | |
| NHE_RS02570 | hypothetical protein | 711 | Hypothetical | General | |
| | | | proteins | | |
| NHE_RS03950 | hypothetical protein | 285 | Hypothetical | General | Ca. Neoehrlichia |
| | | | proteins | | lotoris (α- |
| | | | | | Proteobacteria, 1e⁻³¹) |
| NHE_RS01550 | hypothetical protein | 93 | Hypothetical | General | |
| | | | proteins | | |
| NHE_RS03860 | hypothetical protein | 126 | Hypothetical | General | |
| | | | proteins | | |
| NHE_RS00335 | hypothetical protein | 92 | Hypothetical | General | |
| | | | proteins | | |
| NHE_RS00435 | hypothetical protein | 111 | Hypothetical | General | |
| | | | proteins | | |
| NHE_RS00755 | hypothetical protein | 74 | Hypothetical | General | |
| | | | proteins | | |
| NHE_RS00365 | hypothetical protein | 260 | Hypothetical | General | |
| | | | proteins | | |
| NHE_RS02575 | hypothetical protein | 561 | Hypothetical | General | |
| | | | proteins | | |
| NHE_RS04070 | hypothetical protein | 441 | Hypothetical | General | |
| | | | proteins | | |
| NHE_RS01935 | hypothetical protein | 182 | Hypothetical | General | |
| | | | proteins | | |
| NHE_RS03865 | hypothetical protein | 70 | Hypothetical | General | |
| | | | proteins | | |
| NHE_RS00340 | hypothetical protein | 247 | Hypothetical | General | |
| | | | proteins | | |
| NHE_RS00865 | hypothetical protein | 141 | Hypothetical | General | |
| | | | proteins | | |
| NHE_RS03180 | hypothetical protein | 103 | Hypothetical | General | |
| | | | proteins | | |
| NHE_RS00540 | hypothetical protein | 373 | Hypothetical | General | |
| | | | proteins | | |
| NHE_RS00415 | hypothetical protein | 277 | Hypothetical | General | |
| | | | proteins | | |
| NHE_RS02580 | hypothetical protein | 699 | Hypothetical | General | |
| | | | proteins | | |
| NHE_RS04100 | hypothetical protein | 101 | Hypothetical | General | |
| | | | proteins | | |
| NHE_RS02145 | hypothetical protein | 93 | Hypothetical | General | |
| | | | proteins | | |
| NHE_RS00345 | hypothetical protein | 201 | Hypothetical | General | |
| | | | proteins | | |
| NHE_RS00440 | hypothetical protein | 163 | Hypothetical | General | |
| | | | proteins | | |
| NHE_RS00445 | hypothetical protein | 152 | Hypothetical | General | |
| | | | proteins | | |

¹N. helminthoeca-specific proteins were identified by comparison with N. sennetsu and N. risticii protein databases using BLASTP algorithm with E-value <1e−10.
² N. helminthoeca-specific proteins were blasted against NCBI protein database NR excluding Neorickettsia spp. with E-value <1e−10. The species, class, and E-value of the top matches to the N. helminthoeca proteins were listed. Blank fields, no matches were identified based on the search criteria.

TABLE 7
N. risticii-specific proteins compared to N. helminthoeca and N. sennetsu ¹

| | | Protein | | |
|---|---|---|---|---|
| Locus_ID | Protein Name | Length | Main Role | Sub Role |
| NRI_RS00085 | hypothetical protein | 83 | Unknown function | General |
| NRI_RS00095 | hypothetical protein | 76 | Unknown function | General |
| NRI_RS00240 | hypothetical protein | 60 | Unknown function | General |
| NRI_RS00315 | hypothetical protein | 219 | Unknown function | General |
| NRI_RS00325 | hypothetical protein | 62 | Unknown function | General |
| NRI_RS00365 | hypothetical protein | 210 | Unknown function | General |
| NRI_RS00370 | hypothetical protein | 208 | Unknown function | General |
| NRI_RS00415 | hypothetical protein | 74 | Unknown function | General |
| NRI_RS00440 | hypothetical protein | 111 | Unknown function | General |
| NRI_RS00460 | hypothetical protein | 81 | Unknown function | General |
| NRI_RS00485 | hypothetical protein | 82 | Unknown function | General |
| NRI_RS00615 | hypothetical protein | 205 | Unknown function | General |
| NRI_RS00770 | hypothetical protein | 76 | Unknown function | General |
| NRI_RS01090 | hypothetical protein | 91 | Unknown function | General |
| NRI_RS01340 | hypothetical protein | 63 | Unknown function | General |
| NRI_RS01900 | hypothetical protein | 60 | Unknown function | General |
| NRI_RS02350 | hypothetical protein | 104 | Unknown function | General |
| NRI_RS02630 | hypothetical protein | 61 | Unknown function | General |
| NRI_RS02740 | hypothetical protein | 118 | Unknown function | General |
| NRI_RS03370 | hypothetical protein | 65 | Unknown function | General |
| NRI_RS03385 | hypothetical protein | 60 | Unknown function | General |
| NRI_RS03470 | hypothetical protein | 59 | Unknown function | General |
| NRI_RS00320 | conserved hypothetical protein | 352 | Hypothetical proteins | Conserved |
| NRI_RS00380 | conserved hypothetical protein | 179 | Hypothetical proteins | Conserved |
| NRI_RS02090 | conserved hypothetical protein | 77 | Hypothetical proteins | Conserved |
| NRI_RS02530 | conserved hypothetical protein | 310 | Hypothetical proteins | Conserved |
| NRI_RS02730 | conserved hypothetical protein | 278 | Hypothetical proteins | Conserved |
| NRI_RS03680 | conserved hypothetical protein | 71 | Hypothetical proteins | Conserved |

¹ N. risticii-specific proteins were identified by comparison with N. helminthoeca and N. sennetsu protein databases using Blastp algorithm with E-value <1e⁻¹⁰.

TABLE 8
N. sennetsu-specific proteins compared to N. helminthoeca and N. risticii ¹

| | | Protein | | |
|---|---|---|---|---|
| Locus_ID | Protein Name | Length | Main Role | Sub Role |
| NSE_RS00095 | hypothetical protein | 85 | Unknown function | General |
| NSE_RS00100 | hypothetical protein | 92 | Unknown function | General |
| NSE_RS00120 | hypothetical protein | 69 | Unknown function | General |
| NSE_RS00325 | hypothetical protein | 72 | Unknown function | General |
| NSE_RS00330 | hypothetical protein | 118 | Unknown function | General |
| NSE_RS00370 | hypothetical protein | 180 | Unknown function | General |
| NSE_RS00425 | hypothetical protein | 79 | Unknown function | General |
| NSE_RS00545 | hypothetical protein | 59 | Unknown function | General |
| NSE_RS00565 | hypothetical protein | 335 | Unknown function | General |
| NSE_RS00575 | hypothetical protein | 72 | Unknown function | General |
| NSE_RS00720 | hypothetical protein | 70 | Unknown function | General |
| NSE_RS01695 | hypothetical protein | 59 | Unknown function | General |
| NSE_RS01705 | hypothetical protein | 62 | Unknown function | General |
| NSE_RS01715 | hypothetical protein | 132 | Unknown function | General |
| NSE_RS01960 | hypothetical protein | 59 | Unknown function | General |
| NSE_RS02045 | hypothetical protein | 83 | Unknown function | General |
| NSE_RS03065 | hypothetical protein | 64 | Unknown function | General |
| NSE_RS03225 | hypothetical protein | 62 | Unknown function | General |
| NSE_RS03270 | hypothetical protein | 69 | Unknown function | General |
| NSE_RS03305 | hypothetical protein | 77 | Unknown function | General |
| NSE_RS03385 | hypothetical protein | 68 | Unknown function | General |
| NSE_RS03710 | hypothetical protein | 61 | Unknown function | General |
| NSE_RS03795 | hypothetical protein | 100 | Unknown function | General |

¹ N. sennetsu-specific proteins were identified by comparison with N. helminthoeca and N. risticii protein databases using BLASTP algorithm with E-value <1e−10.

TABLE 9
Amino acid and cofactor biosynthesis in Family Anaplasmataceae

| | | | | | |
|---|---|---|---|---|---|
| Organisms ¹ | NHO | NRI | NES | APH | ECH |

Amino Acids: ²

| | | | | | |
|---|---|---|---|---|---|
| Alanine | + | + | + | + | + |
| Arginine | − | − | − | + ³ | + |
| Asparagine | − | − | − | + | + |
| Aspartate | + | + | + | + | + |
| Cysteine | − | − | − | − | − |
| Glycine | + | + | + | + | + |
| Glutamate ⁴ | + | + | + | + | + |
| Glutamine | + | + | + | + | + |
| Histidine | − | − | − | − | − |
| Leucine | − | − | − | − | − |
| Lysine | − ⁵ | − | − | − | + |
| Isoleucine | − | − | − | − | − |
| Methionine | − | − | − | − | − |
| Phenylalanine | − | − | − | − | − |
| Proline | − | − | − | − | − |
| Serine | − | − | − | − | − |
| Threonine | − | − | − | − | − |
| Tryptophan | − | − | − | − | − |
| Tyrosine | − | − | − | − | − |
| Valine | − | − | − | − | − |

Cofactors:

| | | | | | |
|---|---|---|---|---|---|
| Biotin | + | + | + | + | + |
| FAD | + | + | + | + | + |
| Folate | + | + | + | + | + |
| Lipoate | + | + | + | + | + |
| NAD | + | + | + | + | + |
| CoA ⁶ | + | + | + | + | + |
| Protoheme | + | + | + | + | + |
| Pyridoxine phosphate (Vitamin B6) | + | + | + | + | + |
| Thiamine | + | + | + | + | + |
| Ubiquinone | + | + | + | + | + |

¹ Abbreviations: ECH, Ehrlichia chaffeensis Arkansas; APH, Anaplasma phagocytophilum HZ; NSE, N. sennetsu Miyayama; NRI, N. risticii Illinois; NHO. N. helminthoeca Oregon.
² Biosynthesis for these AAs in N. helminthoeca are converted from other AAs or metabolic intermediates.
³ Only partial enzymes are identified in Arginine biosynthesis pathway in APH.
⁴ Ech and APH can convert Pro to Glu through PutA (bifunctional proline dehydrogenase/pyrroline-5-carboxylate dehydrogenase). All Anaplasmataceae can convert Gln to Glu by CarA/B (carbamoyl phosphate synthase) or GS/PH (bifunctional glutamate synthase subunit beta/2-polyprenylphenol hydroxylase).
⁵ N. helminthoeca encodes complete pathways to synthesize meso- 2,6-diaminopimelate (mDAP) from L-Asp, but lacks diaminopimelate decarboxylase (LysA) at the last step to produce lysine.
⁶ ECH and APH can synthesize CoA from pantothenate, however, all Neorickettsia spp. can only convert 4′-phosphopantetheine to CoA.

TABLE 10
Potential pathogenic genes in Neorickettsia species

| | | | |
|---|---|---|---|
| Organisms ¹ | NHO | NRI | NSE |

Type I Secretion System (T1SS):

| | | | |
|---|---|---|---|
| ATP-binding cassette (ABC) | + | + | + |
| transporter HlyB | | | |
| Membrane fusion protein (MFP) | + | + | + |
| HlyD | | | |
| Outer membrane channel protein | + | + | + |
| TolC | | | |

TAT Pathway:

| | | | |
|---|---|---|---|
| twin-arginine translocation protein, | + | + | + |
| TatA/E family | | | |
| twin-arginine translocation protein, | + | + | + |
| TatB | | | |
| Sec-independent protein translocase | + | + | + |
| TatC | | | |

Type IV Secretion System (T4SS):

| | | | |
|---|---|---|---|
| VirB1 | − | − | − |
| VirB2 | + (3) | + (2) | + (2) |
| VirB3 | + | + | + |
| VirB4 | + (2) | + (2) | + (2) |
| VirB5 | − | − | − |
| VirB6 | + (4) | + (4) | + (4) |
| VirB7 | + | + | + |
| VirB8 | + (2) | + (2) | + (2) |
| VirB9 | + (2) | + (2) | + (2) |
| VirB10 | + | + | + |
| VirB11 | + | + | + |
| VirD4 | + | + | + |

Two-component Systems:

| | | | |
|---|---|---|---|
| PleC/PleD ² | + | + | + |
| CckA/CtrA | + | + | + |
| NtrY/NtrX | − | − | − |

Putative Secreted Effectors:

| | | | |
|---|---|---|---|
| Ankyrin-repeat domain proteins | 4 | 4 | 3 |

¹ Numbers inside parentheses indicate the copy numbers of the genes; otehrwise, only a single copy is present. Abbreviations: NHO, N. helminthoeca Oregon; NRI, N. risticii Illinois; NSE, N. sennetsu Miyayama.
² All Neorickettsia spp. encodes two copies of sensor histidine kindase PleC.

TABLE 11
Putative Transporters of N. helminthoeca

| | | | Gene | Transporter Family/ | |
|---|---|---|---|---|---|
| Locus ID | Protein Name | Gene | Family | Subfamily | Substrate/Function |
| NHE_RS00575 | sodium: alanine | | AGCS | The Alanine or | sodium ion: alanine |
| | symporter family | | | Glycine: Cation | symporter |
| | protein | | | Symporter (AGCS) | |
| | | | | Family | |
| NHE_RS00175 | ABC-type | | ABC | The ATP-binding | protease secretion |
| | protease/lipase | | | Cassette (ABC) | |
| | transport system | | | Superfamily/ABC + | |
| | | | | membrane | |
| NHE_RS01715 | ABC-type | | ABC | The ATP-binding | multidrug |
| | multidrug | | | Cassette (ABC) | |
| | transport system | | | Superfamily/ABC + | |
| | MdlB | | | membrane | |
| NHE_RS02960 | heme ABC | CcmA | ABC | The ATP-binding | heme |
| | exporter ATP- | | | Cassette (ABC) | |
| | binding protein | | | Superfamily/binding | |
| | CcmA | | | | |
| NHE_RS00600 | ccmB family | CcmB | ABC | The ATP-binding | heme export |
| | protein | | | Cassette (ABC) | |
| | | | | Superfamily/ | |
| | | | | membrane | |
| NHE_RS02125 | heme exporter | CcmC | ABC | The ATP-binding | heme export |
| | protein, CcmC | | | Cassette (ABC) | |
| | family | | | Superfamily/ | |
| | | | | membrane | |
| NHE_RS00045 | iron-binding | FbpA | ABC | The ATP-binding | iron(III) |
| | protein FbpA | | | Cassette (ABC) | |
| | | | | Superfamily/Binding | |
| NHE_RS01265 | putative | FbpB | ABC | The ATP-binding | iron(III) |
| | transporter | | | Cassette (ABC) | |
| | | | | Superfamily/ | |
| | | | | membrane | |
| NHE_RS01995 | ABC-type | FbpC | ABC | The ATP-binding | Polyamine or iron(III) |
| | Fe3+/spermidine/ | | | Cassette (ABC) | |
| | putrescine transport | | | Superfamily/binding | |
| | systems | | | | |
| NHE_RS01315 | ABC-type | | ABC | The ATP-binding | lipoprotein |
| | lipoprotein export | | | Cassette (ABC) | |
| | system | | | Superfamily/binding | |
| NHE_RS01370 | CBS domain | | ABC | The ATP-binding | glycine betaine |
| | protein, putative | | | Cassette (ABC) | |
| | | | | Superfamily/binding | |
| NHE_RS02220 | inosine-5′- | | ABC | The ATP-binding | glycine betaine |
| | monophosphate | | | Cassette (ABC) | |
| | dehydrogenase | | | Superfamily/binding | |
| NHE_RS02955 | ABC transporter | | ABC | The ATP-binding | phosphate |
| | ATP-binding | | | Cassette (ABC) | |
| | protein | | | Superfamily/binding | |
| NHE_RS01990 | phosphate ABC | PstA | ABC | The ATP-binding | phosphate |
| | transporter, | | | Cassette (ABC) | |
| | permease protein | | | Superfamily/ | |
| | PstA | | | membrane | |
| NHE_RS03450 | phosphate ABC | PstB | ABC | The ATP-binding | phosphate |
| | transporter ATP- | | | Cassette (ABC) | |
| | binding protein | | | Superfamily/binding | |
| NHE_RS00795 | phosphate ABC | PstC | ABC | The ATP-binding | phosphate |
| | transporter, | | | Cassette (ABC) | |
| | permease protein | | | Superfamily/ | |
| | PstC | | | membrane | |
| NHE_RS03695 | ABC transporter | | ABC | The ATP-binding | lipid A |
| | (iron-sulfur | | | Cassette (ABC) | |
| | clusters) | | | Superfamily/binding | |
| NHE_RS04010 | conserved | | ABC | The ATP-binding | toluene tolerance |
| | hypothetical | | | Cassette (ABC) | |
| | protein | | | Superfamily/binding | |
| NHE_RS02235 | mce-related | | ABC | The ATP-binding | toluene tolerance |
| | protein | | | Cassette (ABC) | |
| | | | | Superfamily/binding | |
| | | | | protein | |
| NHE_RS04005 | putative VacJ | | ABC | The ATP-binding | ? |
| | lipoprotein | | | Cassette (ABC) | |
| | | | | Superfamily/binding | |
| | | | | protein | |
| NHE_RS00530 | lipoprotein | | ABC | The ATP-binding | lipoprotein releasing |
| | releasing system | | | Cassette (ABC) | |
| | transmembrane | | | Superfamily/ | |
| | protein LolE | | | membrane | |
| NHE_RS02950 | ABC transporter | | ABC | The ATP-binding | toluene tolerance |
| | permease protein | | | Cassette (ABC) | |
| | | | | Superfamily/ | |
| | | | | membrane | |
| NHE_RS00740 | permease, PerM | | AI-2E | The Autoinducer-2 | Autoinducer-2 export |
| | family | | | Exporter (AI-2E) | |
| | | | | Family (Formerly the | |
| | | | | PerM Family, TC | |
| | | | | #9.B.22) | |
| NHE_RS00660 | auxin Efflux | | AEC | The Auxin Efflux | |
| | Carrier | | | Carrier (AEC) Family | |
| NHE_RS01325 | ComEC/Rec2 | | DNA-T | The Bacterial | |
| | family protein | | | Competence-related | |
| | | | | DNA Transformation | |
| | | | | Transporter (DNA-T) | |
| | | | | Family | |
| NHE_RS00335 | hypothetical | | CaCA | The Ca2+: Cation | proton: calcium ion |
| | protein | | | Antiporter (CaCA) | antiporter |
| | | | | Family | |
| NHE_RS00345 | hypothetical | | CDF | The Cation Diffusion | cation efflux |
| | protein | | | Facilitator (CDF) | |
| | | | | Family | |
| NHE_RS01685 | inner membrane | | Oxa1 | The Cytochrome | 60 KD inner |
| | protein, 60 kDa | | | Oxidase Biogenesis | membrane protein |
| | | | | (Oxa1) Family | OxaA homolog |
| NHE_RS00770 | putative | | DAACS | The | proton/sodium |
| | transporter | | | Dicarboxylate/Amino | ion: glutamate/ |
| | | | | Acid: Cation (Na+ or | aspartate symporter |
| | | | | H+) Symporter | |
| | | | | (DAACS) Family | |
| NHE_RS00590 | putative | | DASS | The Divalent | sodium |
| | transporter | | | Anion: Na+ Symporter | ion: dicarboxylate/ |
| | | | | (DASS) Family | sulfate |
| NHE_RS00295 | integral membrane | | DMT | The Drug/Metabolite | drug/metabolite? |
| | protein DUF6 | | | Transporter (DMT) | |
| | | | | Superfamily | |
| NHE_RS03960 | motA/TolQ/ExbB | | Mot/Exb | The H+- or Na+- | |
| | proton channel | | | translocating Bacterial | |
| | family protein | | | Flagellar Motor | |
| | | | | 1ExbBD Outer | |
| | | | | Membrane Transport | |
| | | | | Energizer (Mot/Exb) | |
| NHE_RS00505 | ATP synthase F1, | | F- | The H+- or Na+- | protons |
| | alpha subunit | | ATPase | translocating F-type, V- | |
| | | | | type and A-type | |
| | | | | ATPase (F-ATPase) | |
| | | | | Superfamily | |
| NHE_RS00510 | ATP synthase F1, | | F- | The H+- or Na+- | protons |
| | delta subunit | | ATPase | translocating F-type, V- | |
| | | | | type and A-type | |
| | | | | ATPase (F-ATPase) | |
| | | | | Superfamily | |
| NHE_RS01655 | ATP synthase F0, | | F- | The H+- or Na+- | protons |
| | A subunit | | ATPase | translocating F-type, V- | |
| | | | | type and A-type | |
| | | | | ATPase (F-ATPase) | |
| | | | | Superfamily | |
| NHE_RS01660 | conserved domain | | F- | The H+- or Na+- | protons |
| | protein | | ATPase | translocating F-type, V- | |
| | | | | type and A-type | |
| | | | | ATPase (F-ATPase) | |
| | | | | Superfamily | |
| NHE_RS01665 | ATP synthase F0, | | F- | The H+- or Na+- | protons |
| | B′ chain | | ATPase | translocating F-type, V- | |
| | | | | type and A-type | |
| | | | | ATPase (F-ATPase) | |
| | | | | Superfamily | |
| NHE_RS01670 | putative ATPase | | F- | The H+- or Na+- | protons |
| | F0, B chain | | ATPase | translocating F-type, V- | |
| | | | | type and A-type | |
| | | | | ATPase (F-ATPase) | |
| | | | | Superfamily | |
| NHE_RS02510 | ATP synthase F1, | | F- | The H+- or Na+- | protons |
| | gamma subunit | | ATPase | translocating F-type, V- | |
| | | | | type and A-type | |
| | | | | ATPase (F-ATPase) | |
| | | | | Superfamily | |
| NHE_RS03260 | ATP synthase F1, | | F- | The H+- or Na+- | protons |
| | alpha subunit | | ATPase | translocating F-type, V- | |
| | | | | type and A-type | |
| | | | | ATPase (F-ATPase) | |
| | | | | Superfamily | |
| NHE_RS01690 | CBS domain | | HCC | The HlyC/CorC (HCC) | heavy metal ion |
| | protein, putative | | | Family | |
| NHE_RS00290 | drug resistance | | MFS | The Major Facilitator | multidrug efflux |
| | transporter, | | | Superfamily (MFS) | |
| | Bcr/Cf1A family | | | | |
| NHE_RS03325 | major facilitator | | MFS | The Major Facilitator | multidrug efflux |
| | family transporter | | | Superfamily (MFS) | |
| NHE_RS03475 | putative permease | | MFS | The Major Facilitator | Acetyl-CoA: CoA |
| | | | | Superfamily (MFS) | antiporter |
| NHE_RS03605 | major facilitator | | MFS | The Major Facilitator | glycerol-3-phosphate |
| | family transporter | | | Superfamily (MFS) | |
| NHE_RS01920 | magnesium | | MgtE | The Mg2+ Transporter- | magnesium ion |
| | transporter | | | E (MgtE) Family | |
| NHE_RS03965 | membrane protein, | | MC | The Mitochondrial | |
| | putative | | | Carrier (MC) Family | |
| NHE_RS03970 | membrane protein, | | MC | The Mitochondrial | |
| | putative | | | Carrier (MC) Family | |
| NHE_RS00075 | NADH-quinone | MnhA | CPA3 | The Monovalent Cation | multicomponent |
| | oxidoreductase | | | (K+ or Na+): Proton | sodium ion: proton |
| | chain 1 | | | Antiporter-3 (CPA3) | antiporter |
| | | | | Family | |
| NHE_RS02400 | NADH-quinone | MnhA | CPA3 | The Monovalent Cation | multicomponent |
| | oxidoreductase | | | (K+ or Na+): Proton | sodium ion: proton |
| | chain 1 | | | Antiporter-3 (CPA3) | antiporter |
| | | | | Family | |
| NHE_RS00135 | Domain of | MnhB | CPA3 | The Monovalent Cation | multicomponent |
| | unknown function | | | (K+ or Na+): Proton | sodium ion: proton |
| | (DUF4040) | | | Antiporter-3 (CPA3) | antiporter |
| | | | | Family | |
| NHE_RS00140 | multisubunit | MnhB | CPA3 | The Monovalent Cation | multicomponent |
| | Na+/H+ antiporter, | | | (K+ or Na+): Proton | sodium ion: proton |
| | MnhB subunit | | | Antiporter-3 (CPA3) | antiporter |
| | | | | Family | |
| NHE_RS00130 | monovalent | MnhC | CPA3 | The Monovalent Cation | multicomponent |
| | cation/proton | | | (K+ or Na+): Proton | sodium ion: proton |
| | antiporter, | | | Antiporter-3 (CPA3) | antiporter |
| | MnhC/PhaC | | | Family | |
| | subunit family | | | | |
| NHE_RS02990 | NADH-quinone | MnhD | CPA3 | The Monovalent Cation | multicomponent |
| | oxidoreductase | | | (K+ or Na+): Proton | sodium ion: proton |
| | chain 1 | | | Antiporter-3 (CPA3) | antiporter |
| | | | | Family | |
| NHE_RS02185 | conserved | MnhE | CPA3 | The Monovalent Cation | multicomponent |
| | hypothetical | | | (K+ or Na+): Proton | sodium ion: proton |
| | protein | | | Antiporter-3 (CPA3) | antiporter |
| | | | | Family | |
| NHE_RS00145 | monovalent | MnhG | CPA3 | The Monovalent Cation | multicomponent |
| | cation/proton | | | (K+ or Na+): Proton | sodium ion: proton |
| | antiporter, | | | Antiporter-3 (CPA3) | antiporter |
| | MnhG/PhaG | | | Family | |
| | subunit | | | | |
| NHE_RS00705 | multiple resistance | | CPA3 | The Monovalent Cation | sodium ion: proton |
| | and pH regulation | | | (K+ or Na+): Proton | antiporter |
| | protein F (MrpF/ | | | Antiporter-3 (CPA3) | |
| | PhaF) | | | Family | |
| NHE_RS03770 | glutathione- | | CPA2 | The Monovalent | potassium/sodium |
| | regulated | | | Cation: Proton | ion: proton antiporter |
| | potassium-efflux | | | Antiporter-2 (CPA2) | |
| | system protein | | | Family | |
| NHE_RS02395 | membrane protein, | MviN | MOP | The | virulence factor MviN |
| | MviN family | | | Multidrug/ | |
| | | | | Oligosaccharidyl- | |
| | | | | lipid/Polysaccharide | |
| | | | | (MOP) Flippase | |
| | | | | Superfamily/MVF | |
| NHE_RS03705 | conserved | | OAT | The Organo Anion | organic anion |
| | hypothetical | | | Transporter (OAT) | |
| | protein | | | Family | |
| NHE_RS00745 | transporter, | HAE1 | RND | The Resistance- | multidrug/solvent |
| | AcrB/AcrD/AcrF | | | Nodulation-Cell | efflux (HAE1 |
| | family | | | Division (RND) | subfamily) |
| | | | | Superfamily | |
| NHE_RS03610 | mechanosensitive | | MscS | The Small | small-conductance |
| | ion channel family | | | Conductance | mechanosensitive ion |
| | protein | | | Mechanosensitive Ion | channel |
| | | | | Channel (MscS) | |
| | | | | Family | |
| NHE_RS03065 | putative | | SSS | The Solute: Sodium | sodium ion: proline |
| | sodium: proline | | | Symporter (SSS) | symporter |
| | symporter | | | Family | |
| NHE_RS03385 | membrane protein, | | TerC | The Tellurium Ion | tellurium ion efflux |
| | TerC family | | | Resistance (TerC) | |
| | | | | Family | |
| NHE_RS03590 | trap transporter, | | TRAP-T | The Tripartite ATP- | C4-dicarboxylate |
| | 4tm/12tm fusion | | | independent | |
| | protein | | | Periplasmic | |
| | | | | Transporter (TRAP-T) | |
| | | | | Family | |
| NHE_RS02000 | Twin-arginine | TatA | Tat | The Twin Arginine | protein export |
| | translocation | | | Targeting (Tat) Family | |
| | protein, TatA/E | | | | |
| | family | | | | |
| NHE_RS00490 | twin arginine- | TatC | Tat | The Twin Arginine | protein export |
| | targeting protein | | | Targeting (Tat) Family | |
| | translocase TatC | | | | |
| NHE_RS03165 | type IV secretion | VirB8-1 | IVSP | The Type IV (Conjugal | |
| | system protein | | | DNA-Protein Transfer | |
| | VirB8 (VirB8-1) | | | or VirB) Secretory | |
| | | | | Pathway (IVSP) | |
| | | | | Family | |
| NHE_RS03145 | type IV secretion | VirD4 | IVSP | The Type IV (Conjugal | |
| | system protein | | | DNA-Protein Transfer | |
| | VirD4 | | | or VirB) Secretory | |
| | | | | Pathway (IVSP) | |
| | | | | Family | |
| NHE_RS03335 | signal peptidase I | LepB | IVSP | Protein and peptide | |
| | | | | secretion and | |
| | | | | trafficking family | |
| NHE_RS03675 | type IV secretion | VirB6-2 | YggT | The YggT or Fanciful | potassium ion uptake? |
| | system protein, | | | K+ Uptake-B (FkuB; | |
| | VirB6 family | | | YggT) Family | |
| | (VirB6-2) | | | | |
| NHE_RS03670 | type IV secretion | VirB6-3 | YggT | The YggT or Fanciful | potassium ion uptake? |
| | system protein, | | | K+ Uptake-B (FkuB; | |
| | VirB6 family | | | YggT) Family | |
| | (VirB6-3) | | | | |
| NHE_RS03665 | type IV secretion | VirB6-4 | YggT | The YggT or Fanciful | potassium ion uptake? |
| | system protein, | | | K+ Uptake-B (FkuB; | |
| | VirB6 family | | | YggT) Family | |
| | (VirB6-4) | | | | |

TABLE 12
Genes involved in DNA repair and homologous recombination ¹

| | | | | |
|---|---|---|---|---|
| ECH | APH | NSE | NRI | NHO |

Direct Repair

| | | | | | |
|---|---|---|---|---|---|
| Photolyase | | | NSE_RS03995/ | NRI_RS03480 | |
| | | | NSE_RS04000 ² | | |
| DNA ligase ligA | ECH0301 | APH0138 | NSE_RS02025 | NRI_RS02070 | NHE_RS02080 |

AP Endonuclease

| | | | | | |
|---|---|---|---|---|---|
| Xth | ECH0675 | APH0505 | NSE_RS01685 | NRI_RS01735 | NHE_RS01735 |

Base Excision Repair Glycosylases (BER)

| | | | | | |
|---|---|---|---|---|---|
| 3 mg | ECH0277 | | | | |
| Ung Family 4 | ECH0074 | APH1371 | NSE_RS03805 | NRI_RS03885 | NHE_RS04000 |
| Fpg | ECH0602 | APH0411 | | | |
| Nth | ECH0857 | APH0897 | NSE_RS00975 | NRI_RS01015 | NHE_RS00975 |

Nucleotide Excision Repair (NER)

| | | | | | |
|---|---|---|---|---|---|
| UvrA | ECH0785 | APH0537 | | | |
| UvrB | | APH1367 | | | |
| UvrC | | APH0884 | | | |
| UvrD | ECH0860 | APH0903 | NSE_RS01465 | | NHE_RS01505 |
| UvrD family | ECH0387 | APH0258 | NSE_RS01885 | NRI_RS01930 | NHE_RS01930 |

Transcription Coupling Repair (TCR)

| | | |
|---|---|---|
| Mfd | ECH0250 | APH0107 |

Mismatch Repair (MMR)

| | | | | | |
|---|---|---|---|---|---|
| MutL | ECH0884 | APH0939 | NSE_RS02475 | NRI_RS02535 | |
| MutS | ECH0824 | APH0857 | NSE_RS01390 | NRI_RS01440 | NHE_RS04185 |

Homologous Recombination
RecF Pathway

| | | | | | |
|---|---|---|---|---|---|
| RecF | ECH0076 | APH1409 | NSE_RS00780 | NRI_RS00820 | NHE_RS00775 |
| RecJ | ECH1115 | APH1165 | NSE_RS02895 | NRI_RS02985 | NHE_RS03020 |
| RecO | ECH0536 | APH0736 | NSE_RS01855 | NRI_RS01895 | NHE_RS01900 |
| RecR | ECH0843 | APH0988 | NSE_RS03670 | NRI_RS03755 | NHE_RS03835 |

Recombinase

| | | | | | |
|---|---|---|---|---|---|
| RecA | ECH1109 | APH1354 | NSE_RS02170 | NRI_RS02215 | NHE_RS02250 |

Holliday junction resolution

| | | | | | |
|---|---|---|---|---|---|
| RuvA | ECH0320 | APH0167 | NSE_RS02360 | NRI_RS02410 | NHE_RS02455 |
| RuvB | ECH0319 | APH0166 | NSE_RS02365 | NRI_RS02415 | NHE_RS02460 |
| RuvC | ECH0028 | APH0018 | NSE_RS03885 | NRI_RS03965 | NHE_RS04085 |
| RecG | ECH0062 | APH1298 | NSE_RS02795 | NRI_RS02885 | NHE_RS02915 |

Other recombination

| | |
|---|---|
| RadA | ECH0305 |

Other

| | | | | | |
|---|---|---|---|---|---|
| RadC | ECH0363 | APH0242 | NSE_RS00915 | NRI_RS04065/ | NHE_RS00910 * |
| | | | | NRI_RS04060 * | |
| XseL | ECH0056 | APH1322 | | | |
| XseS | ECH0214 | APH0079 | | | |
| Hu | | APH0784 | NSE_RS02595 | NRI_RS02665 | NHE_RS02715 |
| RmuC | ECH0577 | APH0428 | NSE_RS00485 | NRI_RS00530 | NHE_RS00480 |

¹ Abbreviations: ECH, Ehrlichia chaffeensis Arkansas; APH, Anaplasma phagocytophilum HZ; NSE, N. sennetsu Miyayama; NRI, N. risticii Illinois; NHO, N. helminthoeca Oregon.
² Proteins are truncations due to an internal mutation.

TABLE 13
Lipoprotein Processing Enzymes and Putative Lipoproteins in N. helminthoeca

| | | | |
|---|---|---|---|
| | | Protein | |
| Locus ID | Protein Name | Length | LipoBox Sequences |

Lipoprotein processing enzymes:

| | | | |
|---|---|---|---|
| NHE_RS03645 | prolipoprotein diacylglyceryl transferase (Lgt) | 264 | n/a |
| NHE_RS03900 | signal peptidase II (LspA) | 167 | n/a |
| NHE_RS02065 | apolipoprotein N-acyltransferase (Lnt) | 472 | n/a |

Predicted Lipoproteins:

| | | | | |
|---|---|---|---|---|
| NHE_RS02065 | efflux transporter, RND family, MFP subunit | 342 | IFLCS\|CLKD | (SEQ ID NO: 11) |
| NHE_RS00745 | acriflavine resistance protein AcrB | 1023 | FGSYA\|CFVIP | (SEQ ID NO: 12) |
| NHE_RS01690 | CBS domain protein | 421 | SLLLS\|CVFSG | (SEQ ID NO: 13) |
| NHE_RS01870 | beta-ketoacyl-[acyl-carrier-protein] synthase II | 416 | LGLVT\|CLSSK | (SEQ ID NO: 14) |
| NHE_RS02525 | conserved hypothetical protein | 323 | FSLSS\|CAKRG | (SEQ ID NO: 15) |
| NHE_RS02980 | D-alanyl-D-alanine carboxypeptidase | 284 | SSLAH\|CTSAI | (SEQ ID NO: 16) |
| NHE_RS03040 | outer membrane protein assembly complex YaeT | 744 | LFLDP\|CLAEN | (SEQ ID NO: 17) |
| NHE_RS03070 | pentapeptide repeat domain protein | 552 | CSSAD\|CSHTS | (SEQ ID NO: 18) |
| NHE_RS03100 | conserved hypothetical protein | 304 | LCFAP\|CHSLE | (SEQ ID NO: 19) |
| NHE_RS03665 | type IV secretion system protein VirB6-3 | 1069 | FTFSG\|CDHCE | (SEQ ID NO: 20) |
| NHE_RS03670 | type IV secretion system protein VirB6-3 | 1243 | FLFNG\|CDIEC | (SEQ ID NO: 21) |
| NHE_RS03785 | peptidoglycan-associated lipoprotein (PAL/OmpA) | 200 | LLMSG\|CFKKG | (SEQ ID NO: 22) |
| NHE_RS03940 | BamD lipoprotein | 235 | LVVSG\|CTPGK | (SEQ ID NO: 23) |

Putative lipoprotein was predicted by LipoP 1.0 ″|″ indicate the predicted signal peptidase II cleavage site.

TABLE 14
Proteins with tandem repeats in N. helminthoeca

| | | | | Number |
|---|---|---|---|---|
| | | Location of | Repeat | of |
| Locus ID | Protein Name | Repeats | Length | Repeats |
| NHE_RS00170 | conserved hypothetical protein | 284-346 | 30 | 2 |
| Repeats: | AGPRGEDARANVGDPNLPRSSSLPNPNVSHGQE (SEQ ID NO: 24) | | | |
| NHE_RS00220 | hypothetical protein | 449-788 | 20 | 17 |
| Repeats: | TRSHGDLTEMRKALSREPSP (SEQ ID NO: 25) | | | |
| NHE_RS00965 | 51 kda antigen (P51) | 39-56 | 6 | 3 |
| Repeats: | CGCKKT | | | |
| NHE_RS04180 | conserved hypothetical protein | 219-468 | 25 | 10 |
| Repeats: | VEVQTDAPEEPERSTGAASTQTMSE (SEQ ID NO: 26) | | | |
| NHE_RS01860 | conserved hypothetical protein | 147-209 | 21 | 3 |
| Repeats1: | PIPSAEVAQQPAAEPVQQATE (SEQ ID NO: 27) | | | |
| Repeats2: | VEQGSDDNTGADNIEEAIEPIPPAEVAQQPAAEPVQQATEPIPS (SEQ | | | |
| | ID NO: 28) | | | |
| NHE_RS020604 | hexapeptide transferase family protein | 109-284 | 35 | 5 |
| Repeats: | GEISTGPEAITEATEVQDEVKLNPEVITEASGIVD (SEQ ID NO: 29) | | | |
| NHE_RS02225 | inhibitor of apoptosis-promoting Bax1 family protein | 94-159 | 33 | 2 |
| Repeats: | DRVTSDAMPGIQKGAKSTVVWTADAAGRVGAVML (SEQ ID NO: 30) | | | |
| NHE_RS02060 | hexapeptide transferase family protein | 109-284 | 35 | 5 |
| Repeats: | GEISTGPEAITEATEVDEVLLNPEVITEASGIVD (SEQ ID NO: 31) | | | |
| NHE_RS02305 | RDD family protein | 18-31 | 7 | 2 |
| Repeats: | FPHKVFS (SEQ ID NO: 32) | | | |
| NHE_RS02365 | hypothetical protein | 201-224 | 8 | 3 |
| Repeats: | EIMNTTNK (SEQ ID NO: 33) | | | |
| NHE_RS02540 | conserved hypothetical protein | 346-526 | 36 | 5 |
| Repeats: | SSTGSCRPIAAPILNGASLHGVYTSLFEGNKDPGTV (SEQ ID NO: 34) | | | |
| NHE_RS02570 | hypothetical protein | 478-702 | 75 | 3 |
| Repeats: | LRKVGIKEKPFTGDDLIAELKARIEKRSEKNPGKPTVSDSRKRMVTSD | | | |
| | AKDSKQRETQGEKSGN PRTITTETTLE (SEQ ID NO: 35) | | | |
| NHE_RS02695 | conserved hypothetical protein | 329-02 | 37 | 2 |
| Repeats: | VPATSAVMKSIASTGEGGEVVGLSPTLTKFLKEVGEV (SEQ ID NO: 36) | | | |
| NHE_RS03510 | conserved hypothetical protein | 123-272 | 30 | 5 |
| Repeats: | AKYYSAHRDEILQIESRARDPERECFYG (SEQ ID NO: 37) | | | |
| NHE_RS03520 | hypothetical protein | 52-175 | 30 | 4 |
| Repeats1: | PEKFREYKAKHYSAHRDEILQRRRESRARD (SEQ ID NO:3 8) | | | |
| Repeats2: | KYYSAHRDEILQRRRESRARDPEKEFGYGA (SEQ ID NO: 39) | | | |
| NHE_RS03525 | hypothetical protein | 15-231 | 74 | 3 |
| Repeats1: | KKKAEQPIQGTSSSSAPGPSTADLSTSSGSTTVLAPKRRKLTPEEKRER | | | |
| | NRISQAKYYSAHRDE IIQRQREQRA (SEQ ID NO: 40) | | | |
| Repeats2: | ERFREYKAKHYSAHRDEILQRRRESRARDP (SEQ ID NO: 41) | | | |
| NHE_RS03670 | type IV secretion system protein, VirB6-3 | 1007-1243 | 47 | 5 |
| Repeats: | KPKTGEGMVENPIYESGDPVQGAESTENPYSLRGAEGQEEPIYATVD | | | |
| | (SEQ ID NO: 42) | | | |
| NHE_RS03855 | Neorickettsia strain-specific surface antigen (SSA) | 43-137 | | |
| Repeats: | AAEVLKNTTAGDILKNST (SEQ ID NO: 43) | | | |
| NHE_RS04070 | hypothetical protein | 130-353 | 56 | 4 |
| Repeats: | NAPPESLQIELTLDQSEDSSEKQPITPPQQTEPVSLQHQIEPTAPPEPHK | | | |
| | TEPVTV (SEQ ID NO: 44) | | | |

TABLE 15
Oligenucleotide primers used for cloning N. helminthoeca outer membrane proteins

| | | | Amplicon |
|---|---|---|---|
| Target genes | Primer Sequence (5′→3′) | | size |
| P51 | F: ATAGGCCATGG CTTCTGTAGAGAACCCATCAA | (SEQ ID NO: 45) | 1,422 bp |
| (NHE_RS00965) | R: CTAGAGAATTC GTATATGATACTTTGAGACCTGAAG | (SEQ ID NO: 46) | |
| nsp1 | F: ATAGGCCATGG CGCTTTTCGGAATAAACGC | (SEQ ID NO: 47) | 703 bp |
| (NHE_RS03715) | R: CTAGAGAATTC AATATTCCAAGCTGGATCTTGATTCC | (SEQ ID NO: 48) | |
| nsp2 | F: ATAGGCCATGG CCAAAGTAGAAGAAGCGGCGAATGC | (SEQ ID NO: 49) | 870 bp |
| (NHE_RS03720) | R: CTAGAGCGGCGGC GGCGTCAAGTGAAAAAGTAAC | (SEQ ID NO: 50) | |
| nsp3 | F: ATAGGCCATGG CGCAAGATGCCCTAGAGGATG | (SEQ ID NO: 51) | 624 bp |
| (NHE_RS03725) | R: CTAGAGCGGCCGC ATTCATAGGTAGCATTAG | (SEQ ID NO: 52) | |
| ssa | F: ATAGGCCATGG ATCTGCTTAAGCATGATACCTCAAG | (SEQ ID NO: 53) | 1,002 bp |
| (NHE_RS03855) | R: CTAGAGCGGCCGC TTTTTTGGGGATAGTTATCTCTTTAAGTC | (SEQ ID NO: 54) | |

Underlined sequences indicate restriction enzymes' recognition sites: F, Forward (NcoI); R, Reverse complement (EcoRI for p51 and ssp1, NotI for snp2/3, and ssa). Stop codons and approximate 80 bp from 5′-end of these genes that encodes signal peptides were excluded in the amplicon.

What is claimed is:

1. An immunogenic composition comprising one or more isolated Neorickettsia helminthoeca proteins, or immunogenic fragments or variants thereof, or a fusion protein containing same, and a pharmaceutically acceptable carrier,
wherein said composition is capable of producing antibodies specific to N. helminthoeca in a subject to whom the immunogenic composition has been administered, and
wherein the isolated N. helminthoeca protein is selected from the group consisting of: SEQ ID NO:1, SEQ ID NO:2, SEQ ID NO:3, SEQ ID NO:4, and SEQ ID NO:5.

2. The immunogenic composition of claim 1, wherein the isolated N. helminthoeca protein is SEQ ID NO:1.

3. The immunogenic composition of claim 1, wherein the isolated N. helminthoeca protein is SEQ ID NO:2.

4. The immunogenic composition of claim 1, wherein the isolated N. helminthoeca protein is SEQ ID NO:3.

5. The immunogenic composition of claim 1, wherein the isolated N. helminthoeca protein is SEQ ID NO:4.

6. The immunogenic composition of claim 1, wherein the isolated N. helminthoeca protein is SEQ ID NO:5.

7. The immunogenic composition of claim 1, wherein the subject is a member of the Canidae family.

8. A method of preventing or inhibiting salmon poisoning disease (SPD) in a subject comprising:
administering to the subject an immunogenic composition comprising one or more isolated Neorickettsia helminthoeca proteins, or immunogenic fragments or variants thereof, or a fusion protein containing same, and a pharmaceutically acceptable carrier,
wherein said composition is administered in an amount effective to prevent or inhibit salmon poisoning disease (SPD), and
wherein the isolated N. helminthoeca protein is selected from the group consisting of: SEQ ID NO:1, SEQ ID NO:2, SEQ ID NO:3, SEQ ID NO:4, and SEQ ID NO:5.

9. The method of claim 8, wherein the isolated N. helminthoeca protein is SEQ ID NO:1.

10. The method of claim 8, wherein the isolated N. helminthoeca protein is SEQ ID NO:2.

11. The method of claim 8, wherein the isolated N. helminthoeca protein is SEQ ID NO:3.

12. The method of claim 8, wherein the isolated N. helminthoeca protein is SEQ ID NO:4.

13. The method of claim 8, wherein the isolated N. helminthoeca protein is SEQ ID NO:5.

14. The method of claim 8, wherein the subject is a member of the Canidae family.

15. A method for detecting Neorickettsia helminthoeca infection in a canine subject, comprising assaying a sample from the subject for antibodies specific for a N. helminthoeca protein selected from the group consisting of P51, NSP1, NSP2, NSP3, and SSA.

16. The method of claim 15, wherein the N. helminthoeca protein is P51.

17. The method of claim 15, wherein the N. helminthoeca protein is NSP1.

18. The method of claim 15, wherein the N. helminthoeca protein is NSP2.

19. The method of claim 15, wherein the N. helminthoeca protein is NSP3.

20. The method of claim 15, wherein the N. helminthoeca protein is SSA.
